(19) United States
(12) Patent Application Publication
(10) Pub. No.: US 2022/0233592 A1
(43) Pub. Date: Jul. 28, 2022

(54) TREATMENT OF KIDNEY FAILURE USING EX VIVO REPROGRAMMED IMMUNE CELLS

(71) Applicant: CREATIVE MEDICAL TECHNOLOGIES, INC., Phoenix, AZ (US)

(72) Inventors: Thomas Ichim, San Diego, CA (US); Amit Patel, Salt Lake City, UT (US)

(73) Assignee: CREATIVE MEDICAL TECHNOLOGIES, INC., Phoenix, AZ (US)

(21) Appl. No.: 17/585,356

(22) Filed: Jan. 26, 2022

Related U.S. Application Data
(60) Provisional application No. 63/141,555, filed on Jan. 26, 2021.

(51) Int. Cl. A61K 35/17; C12N 5/0783

(52) U.S. Cl. CPC A61K 35/17; C12N 2502/1358; C12N 5/0637

(57) ABSTRACT

Disclosed are treatment methods, protocols, and compositions of matter useful for treatment of kidney failure. The invention discloses, in one embodiment, administration of immune cells that have been reprogrammed by co-culture with regenerative cells. In one embodiment said regenerative cells are umbilical cord derived mesenchymal stem cells and said immune cells are peripheral blood mononuclear cells. In one embodiment cells are cultured together in the presence of interleukin 2 and/or an mTOR inhibitor. In one embodiment said cells are cultured together in the presence of an anti-CD3 and/or anti-CD28 antibody.

### CROSS-REFERENCE TO RELATED APPLICATIONS

This application claims priority to U.S. Provisional Application Ser. No. 63/141,555, filed on Jan. 26, 2021, which is incorporated by reference in its entirety.

### FIELD OF THE INVENTION

The invention belongs to the field of kidney failure, more specifically the invention belongs to the field of stimulating immunity to damaged kidneys by utilized reprogrammed immune cells.

### BACKGROUND OF THE INVENTION

It is well known that kidney failure presents either as a chronic condition or an acute situation. Chronic kidney disease can progress to end-stage renal failure that causes irreversible glomerular and tubular damage leading to the loss of renal function. Apoptosis, oxidative damage and microvascular rarefaction are responsible for glomerular and tubulointerstitial fibrosis in chronic kidney disease. Acute kidney injury (AKI) is a clinical disorder characterized by the sudden loss of kidney function, and such function includes excreting wastes, maintaining electrolyte and acid-base balance, and maintaining body fluid levels. The clinical signs of AKI are sudden illness, vomiting, anorexia, weight loss, exercise intolerance, and eventually death. AKI presents as an acute deterioration in renal excretory function within hours or days, resulting in the accumulation of “uremic toxins” and, importantly, a rise in the blood levels of potassium, hydrogen and other ions. This accumulation contributes to life threatening multisystem complications such as bleeding, seizures, renal arrhythmias or arrest, and possible volume overload with pulmonary congestion and poor oxygen uptake. The most common cause of AKI is an ischemic insult of the kidney resulting in injury of renal tubular and postglomerular vascular endothelial cells. The principal etiologies for this ischemic form of AKI include intravascular volume contraction, resulting from bleeding, thrombotic events, shock, sepsis, major cardiovascular surgery, arterial stenoses, and others. AKI can be caused nephrotoxic agents such as radiocontrast agents or a significant number of frequently used medications including chemotherapeutic drugs, antibiotics and certain immunosuppressants such as cis-Platinum and cyclosporine. Patients most at risk for all forms of AKI include diabetics, those with underlying kidney, liver, and/or cardiovascular disease, the elderly, recipients of a bone marrow transplant, and those with cancer or other debilitating disorders.

The present disclosure satisfies a long felt need in the art by providing effective prevention and treatment of one or more kidney diseases.

### SUMMARY

Preferred embodiments are directed to methods of preventing, and/or inhibiting, and/or reversing renal failure comprising the steps of: a) identifying a patient suffering from renal failure and/or a patient having undergoing renal fibrosis; b) extracting from said patient immune cells; c) contacting said immune cells with regenerative cells in a manner so that regenerative cells endow onto said immune cells properties capable of inhibiting and/or reversing renal failure and/or renal fibrosis; and d) administering said immune cells into said patient.

Preferred methods include embodiments wherein said renal failure is associated with renal damage.

Preferred methods include embodiments wherein said renal failure is associated with chemotherapy induced tissue damage.

Preferred methods include embodiments wherein said renal failure is associated with fibrotic damage.

Preferred methods include embodiments wherein said renal failure is associated with ischemia/reperfusion injury.

Preferred methods include embodiments wherein said renal failure occurs as a result of ischemia/reperfusion injury as a result of a myocardial infarct.

Preferred methods include embodiments wherein said renal failure is autoimmune mediated.

Preferred methods include embodiments wherein said immune cells are extracted from a patient who is not the recipient.

Preferred methods include embodiments wherein said immune cells are xenogeneic.

Preferred methods include embodiments wherein said immune cells are cord blood derived.

Preferred methods include embodiments wherein said immune cells are derived from pluripotent stem cells.

Preferred methods include embodiments wherein said immune cells are cultured together with said regenerative cells in the presence of an activator of an immune receptor.

Preferred methods include embodiments wherein said immune receptor activates immunotyrosine activation motifs.

Preferred methods include embodiments wherein said immune receptor activates NF-AT.

Preferred methods include embodiments wherein said immune receptor activates NF-kappa B.

Preferred methods include embodiments wherein said immune receptor activates STAT-3.

Preferred methods include embodiments wherein said immune receptor activates STAT-4.

Preferred methods include embodiments wherein said immune receptor activates janus activated kinase.

Preferred methods include embodiments wherein said immune receptor activates MAP-kinase.

Preferred methods include embodiments wherein said immune receptor is TLR. 1

Preferred methods include embodiments wherein said TLR-1 is activated by Pam3CSK4.

Preferred methods include embodiments wherein said immune receptor is TLR-2

Preferred methods include embodiments wherein said TLR-2 is activated by HKLM.

Preferred methods include embodiments wherein said immune receptor is TLR-3.

Preferred methods include embodiments wherein said TLR-3 is activated by Poly:IC.

Preferred methods include embodiments wherein said immune receptor is TLR-4.

Preferred methods include embodiments wherein said TLR-4 is activated by LPS.

Preferred methods include embodiments wherein said TLR-4 is activated by Buprenorphine.

Preferred methods include embodiments wherein said TLR-4 is activated by Carbamazepine.

Preferred methods include embodiments wherein said TLR-4 is activated by Fentanyl.

Preferred methods include embodiments wherein said TLR-4 is activated by Levorphanol.

Preferred methods include embodiments wherein said TLR-4 is activated by Methadone.

Preferred methods include embodiments wherein said TLR-4 is activated by Cocaine.

Preferred methods include embodiments wherein said TLR-4 is activated by Morphine.

Preferred methods include embodiments wherein said TLR-4 is activated by Oxcarbazepine.

Preferred methods include embodiments wherein said TLR-4 is activated by Oxycodone.

Preferred methods include embodiments wherein said TLR-4 is activated by Pethidine.

Preferred methods include embodiments wherein said TLR-4 is activated by Glucuronoxylomannan from Cryptococcus.

Preferred methods include embodiments wherein said TLR-4 is activated by Morphine-3-glucuronide.

Preferred methods include embodiments wherein said TLR-4 is activated by lipoteichoic acid.

Preferred methods include embodiments wherein said TLR-4 is activated by beta.-defensin 2.

Preferred methods include embodiments wherein said TLR-4 is activated by low molecular weight hyaluronic acid.

Preferred methods include embodiments wherein said low molecular weight hyaluronic acid has a molecular weight of <1000 kDa.

Preferred methods include embodiments wherein said low molecular weight hyaluronic acid has a molecular weight of <500 kDa.

Preferred methods include embodiments wherein said low molecular weight hyaluronic acid has a molecular weight of <250 kDa.

Preferred methods include embodiments wherein said low molecular weight hyaluronic acid has a molecular weight of <100 kDa.

Preferred methods include embodiments wherein said TLR-4 is activated by fibronectin EDA.

Preferred methods include embodiments wherein said TLR-4 is activated by snapin.

Preferred methods include embodiments wherein said TLR-4 is activated by tenascin C.

Preferred methods include embodiments wherein said immune receptor is TLR-5.

Preferred methods include embodiments wherein said TLR-5 is activated by flaggelin.

Preferred methods include embodiments wherein said immune receptor is TLR-6.

Preferred methods include embodiments wherein said TLR-6 is activated by FSL-

Preferred methods include embodiments wherein said immune receptor is TLR-7.

Preferred methods include embodiments wherein said TLR-7 is activated by imiquimod.

Preferred methods include embodiments wherein said immune receptor is TLR-8.

Preferred methods include embodiments wherein said TLR-8 is activated by ssRNA40/LyoVec.

Preferred methods include embodiments wherein said immune receptor is TLR-9.

Preferred methods include embodiments wherein said TLR-9 is activated by a CpG oligonucleotide.

Preferred methods include embodiments wherein said TLR-9 is activated by ODN2006.

Preferred methods include embodiments wherein said TLR-9 is activated by Agatolimod.

Preferred methods include embodiments wherein said TLR-9 is activated by ODN2007.

Preferred methods include embodiments wherein said TLR-9 is activated by ODN1668.

Preferred methods include embodiments wherein said TLR-9 is activated by ODN1826.

Preferred methods include embodiments wherein said TLR-9 is activated by ODN BW006.

Preferred methods include embodiments wherein said TLR-9 is activated by ODN D SL01.

Preferred methods include embodiments wherein said TLR-9 is activated by ODN 2395.

Preferred methods include embodiments wherein said TLR-9 is activated by ODN M362.

Preferred methods include embodiments wherein said TLR-9 is activated by ODN SL03.

Preferred methods include embodiments wherein said regenerative cell is a stem cell.

Preferred methods include embodiments wherein said stem cell is a hematopoietic stem cell.

Preferred methods include embodiments wherein said hematopoietic stem cell is capable of generating leukocytic, lymphocytic, thrombocytic and erythrocytic cells when transplanted into an immunodeficient animal.

Preferred methods include embodiments wherein said hematopoietic stem cell expresses interleukin-3 receptor.

Preferred methods include embodiments wherein said hematopoietic stem cell expresses interleukin-1 receptor.

Preferred methods include embodiments wherein said hematopoietic stem cell expresses c-met.

Preferred methods include embodiments wherein said hematopoietic stem cell expresses mpl.

Preferred methods include embodiments wherein said hematopoietic stem cell expresses interleukin-11 receptor.

Preferred methods include embodiments wherein said hematopoietic stem cell expresses G-CSF receptor.

Preferred methods include embodiments wherein said hematopoietic stem cell expresses GM-CSF receptor.

Preferred methods include embodiments wherein said hematopoietic stem cell expresses M-CSF receptor.

Preferred methods include embodiments wherein said hematopoietic stem cell expresses VEGF-receptor.

Preferred methods include embodiments wherein said hematopoietic stem cell expresses c-kit.

Preferred methods include embodiments wherein said hematopoietic stem cell expresses CD33.

Preferred methods include embodiments wherein said hematopoietic stem cell expresses CD133.

Preferred methods include embodiments wherein said hematopoietic stem cell expresses CD34.

Preferred methods include embodiments wherein said hematopoietic stem cell expresses Fas ligand.

Preferred methods include embodiments wherein said hematopoietic stem cell does not express lineage markers.

Preferred methods include embodiments wherein said hematopoietic stem cell does not express CD14.

Preferred methods include embodiments wherein said hematopoietic stem cell does not express CD16.

Preferred methods include embodiments wherein said hematopoietic stem cell does not express CD3.

Preferred methods include embodiments wherein said hematopoietic stem cell does not express CD56.

Preferred methods include embodiments wherein said hematopoietic stem cell does not express CD38.

Preferred methods include embodiments wherein said hematopoietic stem cell does not express CD30.

Preferred methods include embodiments wherein said regenerative cell is a mesenchymal stem cell.

Preferred methods include embodiments wherein said mesenchymal stem cells are naturally occurring mesenchymal stem cells.

Preferred methods include embodiments wherein said mesenchymal stem cells are generated in vitro.

Preferred methods include embodiments wherein said naturally occurring mesenchymal stem cells are tissue derived.

Preferred methods include embodiments wherein said naturally occurring mesenchymal stem cells are derived from a bodily fluid.

Preferred methods include embodiments wherein said tissue derived mesenchymal stem cells are selected from a group comprising of: a) bone marrow; b) perivascular tissue; c) adipose tissue; d) placental tissue; e) amniotic membrane; f) omentum; g) tooth; h) umbilical cord tissue; i) fallopian tube tissue; j) hepatic tissue; k) renal tissue; l) renal tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; v) skeletal muscle tissue; and w) subepithelial umbilical cord tissue.

Preferred methods include embodiments wherein said tissue derived mesenchymal stem cells are isolated from tissues containing cells selected from a group of cells comprising of: endothelial cells, epithelial cells, dermal cells, endodermal cells, mesodermal cells, reprogrammed immune cells, osteocytes, chondrocytes, natural killer cells, dendritic cells, hepatic cells, pancreatic cells, stromal cells, salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue reprogrammed immune cells, corneal keratocytes, tendon reprogrammed immune cells, bone marrow reticular tissue reprogrammed immune cells, nonepithelial reprogrammed immune cells, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, melanocytes, retinal pigmented epithelial cells, oogonia/oocytes, spermatids, spermatocytes, spermatogonium cells, spermatozoa, ovarian follicle cells, Sertoli cells, thymus epithelial cell, and/or interstitial kidney cells.

Preferred methods include embodiments wherein said mesenchymal stem cells are plastic adherent.

Preferred methods include embodiments wherein said mesenchymal stem cells express a marker selected from a group comprising of: a) CD73; b) CD90; and c) CD105.

Preferred methods include embodiments wherein said mesenchymal stem cells lack expression of a marker selected from a group comprising of: a) CD14; b) CD45; and c) CD34.

Preferred methods include embodiments wherein said mesenchymal stem cells from umbilical cord tissue express markers selected from a group comprising of; a) oxidized low density lipoprotein receptor 1, b) chemokine receptor ligand 3; and c) granulocyte chemotactic protein.

Preferred methods include embodiments wherein said mesenchymal stem cells from umbilical cord tissue do not express markers selected from a group comprising of: a) CD117; b) CD31; c) CD34; and CD45;

Preferred methods include embodiments wherein said mesenchymal stem cells from umbilical cord tissue express, relative to a human fibroblast, increased levels of interleukin 8 and reticulon 1.

Preferred methods include embodiments wherein said mesenchymal stem cells from umbilical cord tissue have the potential to differentiate into cells of at least a skeletal muscle, vascular smooth muscle, pericyte or vascular endothelium phenotype.

Preferred methods include embodiments wherein said mesenchymal stem cells from umbilical cord tissue express markers selected from a group comprising of: a) CD10; b) CD13; c) CD44; d) CD73; and e) CD90.

Preferred methods include embodiments wherein said umbilical cord tissue mesenchymal stem cell is an isolated umbilical cord tissue cell isolated from umbilical cord tissue substantially free of blood that is capable of self-renewal and expansion in culture,

Preferred methods include embodiments wherein said umbilical cord tissue mesenchymal stem cells has the potential to differentiate into cells of other phenotypes.

Preferred methods include embodiments wherein said other phenotypes comprise: a) osteocytic; b) adipogenic; and c) chondrogenic differentiation.

Preferred methods include embodiments wherein said cord tissue derived mesenchymal stem cells can undergo at least 20 doublings in culture.

Preferred methods include embodiments wherein said cord tissue derived mesenchymal stem cell maintains a normal karyotype upon passaging.

Preferred methods include embodiments wherein said cord tissue derived mesenchymal stem cell expresses a marker selected from a group of markers comprised of: a) CD10 b) CD13; c) CD44; d) CD73; e) CD90; f) PDGFr-alpha; g) PD-L2; and h) HLA-A,B,C.

Preferred methods include embodiments wherein said cord tissue mesenchymal stem cells does not express one or more markers selected from a group comprising of; a) CD31; b) CD34; c) CD45; d) CD80; e) CD86; f) CD117; g) CD141; h) CD178; i) B7-H2; j) HLA-G and k) HLA-DR,DP,DQ.

Preferred methods include embodiments wherein said umbilical cord tissue-derived cell secretes factors selected from a group comprising of: a) MCP-1; b) MIPlbeta; c) IL-6; d) IL-8; e) GCP-2; f) HGF; g) KGF; h) FGF; i) HB-EGF; j) BDNF; k) TPO; 1) RANTES; and m) TIMP1 114.

Preferred methods include embodiments wherein said umbilical cord tissue derived cells express markers selected from a group comprising of: a) TRA1-60; b) TRA1-81; c) SSEA3; d) SSEA4; and e) NANOG.

Preferred methods include embodiments wherein said umbilical cord tissue-derived cells are positive for alkaline phosphatase staining.

Preferred methods include embodiments wherein said umbilical cord tissue-derived cells are capable of differentiating into one or more lineages selected from a group comprising of; a) ectoderm; b) mesoderm, and; c) endoderm.

Preferred methods include embodiments wherein said bone marrow derived mesenchymal stem cells possess markers selected from a group comprising of: a) CD73; b) CD90; and c) CD105.

Preferred methods include embodiments wherein said bone marrow derived mesenchymal stem cells possess markers selected from a group comprising of: a) LFA-3; b) ICAM-1; c) PECAM-1; d) P-selectin; e) L-selectin; f) CD49b/CD29; g) CD49c/CD29; h) CD49d/CD29; i) CD29; j) CD18; k) CD61; l) 6-19; m) thrombomodulin; n) telomerase; o) CD10; p) CD13; and q) integrin beta.

Preferred methods include embodiments wherein said bone marrow derived mesenchymal stem cell is a mesenchymal stem cell progenitor cell.

Preferred methods include embodiments wherein said mesenchymal progenitor cells are a population of bone marrow mesenchymal stem cells enriched for cells containing STRO-1

Preferred methods include embodiments wherein said mesenchymal progenitor cells express both STRO-1 and VCAM-

Preferred methods include embodiments wherein said STRO-1 expressing cells are negative for at least one marker selected from the group consisting of: a) CBFA-1; b) collagen type II; c) PPAR.gamma2; d) osteopontin; e) osteocalcin; f) parathyroid hormone receptor; g) leptin; h) H-ALBP; i) aggrecan; j) Ki67, and k) glycophorin A.

Preferred methods include embodiments wherein said bone marrow mesenchymal stem cells lack expression of CD14, CD34, and CD45.

Preferred methods include embodiments wherein said STRO-1 expressing cells are positive for a marker selected from a group comprising of: a) VACM-1; b) TKY-1; c) CD146 and; d) STRO-2

Preferred methods include embodiments wherein said bone marrow mesenchymal stem cell express markers selected from a group comprising of: a) CD13; b) CD34; c) CD56 and; d) CD117

Preferred methods include embodiments wherein said bone marrow mesenchymal stem cells do not express CD10.

Preferred methods include embodiments wherein said bone marrow mesenchymal stem cells do not express CD2, CD5, CD14, CD19, CD33, CD45, and DRII.

Preferred methods include embodiments wherein said bone marrow mesenchymal stem cells express CD13, CD34, CD56, CD90, CD117 and nestin, and which do not express CD2, CD3, CD10, CD14, CD16, CD31, CD33, CD45 and CD64.

Preferred methods include embodiments wherein said skeletal muscle stem cells express markers selected from a group comprising of: a) CD13; b) CD34; c) CD56 and; d) CD117

Preferred methods include embodiments wherein said skeletal muscle mesenchymal stem cells do not express CD10.

Preferred methods include embodiments wherein said skeletal muscle mesenchymal stem cells do not express CD2, CD5, CD14, CD19, CD33, CD45, and DRII.

Preferred methods include embodiments wherein said bone marrow mesenchymal stem cells express CD13, CD34, CD56, CD90, CD117 and nestin, and which do not express CD2, CD3, CD10, CD14, CD16, CD31, CD33, CD45 and CD64.

Preferred methods include embodiments wherein said subepithelial umbilical cord derived mesenchymal stem cells possess markers selected from a group comprising of; a) CD29; b) CD73; c) CD90; d) CD166; e) SSEA4; f) CD9; g) CD44; h) CD146; and i) CD105

Preferred methods include embodiments wherein said subepithelial umbilical cord derived mesenchymal stem cells do not express markers selected from a group comprising of; a) CD45; b) CD34; c) CD14; d) CD79; e) CD106; f) CD86; g) CD80; h) CD19; i) CD117; j) Stro-1 and k) HLA-DR.

Preferred methods include embodiments wherein said subepithelial umbilical cord derived mesenchymal stem cells express CD29, CD73, CD90, CD166, SSEA4, CD9, CD44, CD146, and CD105.

Preferred methods include embodiments wherein said subepithelial umbilical cord derived mesenchymal stem cells do not express CD45, CD34, CD14, CD79, CD106, CD86, CD80, CD19, CD117, Stro-1, and HLA-DR.

Preferred methods include embodiments wherein said subepithelial umbilical cord derived mesenchymal stem cells are positive for SOX2.

Preferred methods include embodiments wherein said subepithelial umbilical cord derived mesenchymal stem cells are positive for OCT4.

Preferred methods include embodiments wherein said subepithelial umbilical cord derived mesenchymal stem cells are positive for OCT4 and SOX2.

Preferred methods include embodiments wherein said immune cells are T cells.

Preferred methods include embodiments wherein said T cells are CD4 cells.

Preferred methods include embodiments wherein said T cells are CD8 cells.

Preferred methods include embodiments wherein said T cells are NKT cells.

Preferred methods include embodiments wherein said T cells are gamma delta T cells.

Preferred methods include embodiments wherein said T cells are Th1 cells.

Preferred methods include embodiments wherein said Th1 cells have a propensity for producing interferon gamma over interleukin 4 upon stimulation via the CD3 protein.

Preferred methods include embodiments wherein said Th1 cells express STAT4.

Preferred methods include embodiments wherein said Th1 cells express STAT1.

Preferred methods include embodiments wherein said Th1 cells express T-bet.

Preferred methods include embodiments wherein said Th1 cells express CCR1.

Preferred methods include embodiments wherein said Th1 cells express CCR5.

Preferred methods include embodiments wherein said Th1 cells express CXCR3.

Preferred methods include embodiments wherein said Th1 cells express CD119.

Preferred methods include embodiments wherein said Th1 cells express interferon gamma receptor II.

Preferred methods include embodiments wherein said Th1 cells express IL-18 receptor.

Preferred methods include embodiments wherein said Th1 cells express IL-12 receptor.

Preferred methods include embodiments wherein said Th1 cells express IL-27 receptor.

Preferred methods include embodiments wherein said T cells are Th2 cells.

Preferred methods include embodiments wherein said Th2 cells have a proclivity to produce more interleukin-4 than interferon gamma upon stimulation via CD3.

Preferred methods include embodiments wherein said Th2 cells express GATA-3.

Preferred methods include embodiments wherein said Th2 cells express IRF-4.

Preferred methods include embodiments wherein said Th2 cells express STAT5.

Preferred methods include embodiments wherein said Th2 cells express STAT6.

Preferred methods include embodiments wherein said Th2 cells express CCR3.

Preferred methods include embodiments wherein said Th2 cells express CCR4.

Preferred methods include embodiments wherein said Th2 cells express CCR8.

Preferred methods include embodiments wherein said Th2 cells express CXCR4.

Preferred methods include embodiments wherein said Th2 cells express interleukin-4 receptor.

Preferred methods include embodiments wherein said Th2 cells express interleukin-33 receptor.

Preferred methods include embodiments wherein said T cells are Th9 cells.

Preferred methods include embodiments wherein said Th9 cell produces interleukin-9.

Preferred methods include embodiments wherein said Th9 cell expresses IRF4.

Preferred methods include embodiments wherein said Th9 cell expresses PU.1.

Preferred methods include embodiments wherein said Th9 cell secretes CCL17.

Preferred methods include embodiments wherein said Th9 cell secretes CCL22.

Preferred methods include embodiments wherein said Th9 cell secretes IL-10.

Preferred methods include embodiments wherein said Th9 cell expresses TGF-beta receptor II.

Preferred methods include embodiments wherein said T cell is a follicular helper T cell.

Preferred methods include embodiments wherein said follicular helper T cell expresses bcl-6.

Preferred methods include embodiments wherein said follicular helper T cell expresses c-maf.

Preferred methods include embodiments wherein said follicular helper T cell expresses stat-3.

Preferred methods include embodiments wherein said follicular helper T cell secretes CXCL-13.

Preferred methods include embodiments wherein said follicular helper T cell secretes interferon gamma.

Preferred methods include embodiments wherein said follicular helper T cell secretes interleukin-4.

Preferred methods include embodiments wherein said follicular helper T cell secretes IL-10.

Preferred methods include embodiments wherein said follicular helper T cell secretes IL-17A.

Preferred methods include embodiments wherein said follicular helper T cell secretes IL-17F.

Preferred methods include embodiments wherein said follicular helper T cell secretes IL-2

Preferred methods include embodiments wherein said follicular helper T cell expresses BTLA-4.

Preferred methods include embodiments wherein said follicular helper T cell secretes CD40 ligand.

Preferred methods include embodiments wherein said follicular helper T cell expresses CD57.

Preferred methods include embodiments wherein said follicular helper T cell expresses CD84.

Preferred methods include embodiments wherein said follicular helper T cell expresses CXCR-4.

Preferred methods include embodiments wherein said follicular helper T cell expresses CXCR-5.

Preferred methods include embodiments wherein said follicular helper T cell expresses ICOS.

Preferred methods include embodiments wherein said follicular helper T cell expresses IL-6 receptor.

Preferred methods include embodiments wherein said follicular helper T cell expresses IL-21 receptor.

Preferred methods include embodiments wherein said follicular helper T cell expresses CD10.

Preferred methods include embodiments wherein said follicular helper T cell expresses OX40.

Preferred methods include embodiments wherein said follicular helper T cell expresses PD-

Preferred methods include embodiments wherein said follicular helper T cell expresses CD150.

Preferred methods include embodiments wherein said T cell is a Th17 cell.

Preferred methods include embodiments wherein said Th17 cell secretes interleukin-17A.

Preferred methods include embodiments wherein said Th17 cell secretes interleukin-17F.

Preferred methods include embodiments wherein said Th17 cell secretes IL-21.

Preferred methods include embodiments wherein said Th17 cell secretes IL-26.

Preferred methods include embodiments wherein said Th17 cell secretes CCL20.

Preferred methods include embodiments wherein said Th17 cell expresses BATF.

Preferred methods include embodiments wherein said Th17 cell expresses IRF4.

Preferred methods include embodiments wherein said Th17 cell expresses ROR alpha.

Preferred methods include embodiments wherein said Th17 cell expresses ROR gamma.

Preferred methods include embodiments wherein said Th17 cell expresses STAT5.

Preferred methods include embodiments wherein said Th17 cell expresses CCR4.

Preferred methods include embodiments wherein said Th17 cell expresses CCR6.

Preferred methods include embodiments wherein said Th17 cell expresses IL-1 receptor.

Preferred methods include embodiments wherein said Th17 cell expresses IL-6 receptor alpha.

Preferred methods include embodiments wherein said Th17 cell expresses IL-21 receptor.

Preferred methods include embodiments wherein said Th17 cell expresses IL-23 receptor.

Preferred methods include embodiments wherein said T cell is a Th22 cell.

Preferred methods include embodiments wherein said Th22 cell secretes IL-10.

Preferred methods include embodiments wherein said Th22 cell secretes IL-13.

Preferred methods include embodiments wherein said Th22 cell secretes FGF-1.

Preferred methods include embodiments wherein said Th22 cell secretes FGF-2.

Preferred methods include embodiments wherein said Th22 cell secretes FGF-5.

Preferred methods include embodiments wherein said Th22 cell secretes IL-21.

Preferred methods include embodiments wherein said Th22 cell secretes IL-22.

Preferred methods include embodiments wherein said Th22 cell expresses AHR.

Preferred methods include embodiments wherein said Th22 cell expresses batf.

Preferred methods include embodiments wherein said Th22 cell expresses STAT-3

Preferred methods include embodiments wherein said Th22 cell expresses CCR4.

Preferred methods include embodiments wherein said Th22 cell expresses CCR6.

Preferred methods include embodiments wherein said Th22 cell expresses CCR10.

Preferred methods include embodiments wherein said Th22 cell expresses IL-6 receptor.

Preferred methods include embodiments wherein said Th22 cell expresses TGF-beta receptor II.

Preferred methods include embodiments wherein said Th22 cell expresses TNF receptor

Preferred methods include embodiments wherein said T cell is a T regulatory cell.

Preferred methods include embodiments wherein said T regulatory cell expresses foxp-3.

Preferred methods include embodiments wherein said T regulatory cell expresses Helios.

Preferred methods include embodiments wherein said T regulatory cell expresses STAT5.

Preferred methods include embodiments wherein said T regulatory cell expresses CD5.

Preferred methods include embodiments wherein said T regulatory cell expresses CD25.

Preferred methods include embodiments wherein said T regulatory cell expresses CD39.

Preferred methods include embodiments wherein said T regulatory cell expresses CD105.

Preferred methods include embodiments wherein said T regulatory cell expresses IL-7 receptor.

Preferred methods include embodiments wherein said T regulatory cell expresses CTLA-4.

Preferred methods include embodiments wherein said T regulatory cell expresses folate receptor.

Preferred methods include embodiments wherein said T regulatory cell expresses CD223

Preferred methods include embodiments wherein said T regulatory cell expresses LAP.

Preferred methods include embodiments wherein said T regulatory cell expresses GARP.

Preferred methods include embodiments wherein said T regulatory cell expresses Neuropilin.

Preferred methods include embodiments wherein said T regulatory cell expresses CD134.

Preferred methods include embodiments wherein said T regulatory cell expresses CD62 ligand.

Preferred methods include embodiments wherein said T regulatory cell secretes IL-10.

Preferred methods include embodiments wherein said T regulatory cell secretes TGF-alpha.

Preferred methods include embodiments wherein said T regulatory cell secretes TGF-beta.

Preferred methods include embodiments wherein said T regulatory cell secretes soluble TNF receptor p55.

Preferred methods include embodiments wherein said T regulatory cell secretes soluble TNF receptor p75.

Preferred methods include embodiments wherein said T regulatory cell secretes IL-2.

Preferred methods include embodiments wherein said T regulatory cell secretes soluble HLA-G.

Preferred methods include embodiments wherein said T regulatory cell secretes soluble Fas ligand.

Preferred methods include embodiments wherein said T regulatory cell secretes IL-35.

Preferred methods include embodiments wherein said T regulatory cell secretes VEGF.

Preferred methods include embodiments wherein said T regulatory cell secretes HGF.

Preferred methods include embodiments wherein said T regulatory cell secretes FGF1.

Preferred methods include embodiments wherein said T regulatory cell secretes FGF2.

Preferred methods include embodiments wherein said T regulatory cell secretes FGF5.

Preferred methods include embodiments wherein said T regulatory cell secretes Galectin 1.

Preferred methods include embodiments wherein said T regulatory cell secretes Galectin 9.

Preferred methods include embodiments wherein said T regulatory cell secretes IL-20.

Preferred methods include embodiments wherein said T regulatory cell expresses perforin.

Preferred methods include embodiments wherein said T regulatory cell expresses granzyme.

Preferred methods include embodiments wherein said T regulatory cell inhibits activation of a conventional T cell.

Preferred methods include embodiments wherein said conventional T cell does not express CD25.

Preferred methods include embodiments wherein said conventional T cell is activated by ligation of CD3.

Preferred methods include embodiments wherein said T conventional T cell is activated by ligation of CD3 and CD28.

Preferred methods include embodiments wherein said activation of said conventional T cell is proliferation.

Preferred methods include embodiments wherein said activation of said conventional T cell is cytokine secretion.

Preferred methods include embodiments wherein said cytokine is IL-2.

Preferred methods include embodiments wherein said cytokine is IL-4.

Preferred methods include embodiments wherein said cytokine is IL-6.

Preferred methods include embodiments wherein said cytokine is IL-10.

Preferred methods include embodiments wherein said cytokine is IL-12.

Preferred methods include embodiments wherein said cytokine is IL-7.

Preferred methods include embodiments wherein said cytokine is IL-13.

Preferred methods include embodiments wherein said cytokine is IL-15.

Preferred methods include embodiments wherein said cytokine is IL-18.

Preferred methods include embodiments wherein said immune cells are peripheral blood mononuclear cells.

Preferred methods include embodiments wherein said peripheral blood mononuclear cells are treated with an immune modulatory agent prior to contacting with said regenerative cells.

Preferred methods include embodiments wherein said immune modulatory agent is ultraviolet light.

Preferred methods include embodiments wherein said immune modulatory agent is HGF.

Preferred methods include embodiments wherein said immune modulatory agent is oxytocin.

Preferred methods include embodiments wherein said immune modulatory agent is NGF.

Preferred methods include embodiments wherein said immune modulatory agent is FGF-

Preferred methods include embodiments wherein said immune modulatory agent is FGF-2.

Preferred methods include embodiments wherein said immune modulatory agent is a toll like receptor activator.

Preferred methods include embodiments wherein oxytocin is administered to peripheral blood mononuclear cells in vitro for a period of 1 minute to 4 weeks.

Preferred methods include embodiments wherein oxytocin is administered to peripheral blood mononuclear cells in vitro for a period of 2 hours to 1 week.

Preferred methods include embodiments wherein oxytocin is administered to said reprogrammed immune cells in vitro for a period of 24 hours to 72 hours.

Preferred methods include embodiments wherein said oxytocin is administered at a concentration of 10 nM-10 uM.

Preferred methods include embodiments wherein said oxytocin is administered at a concentration of 100 nM-1 uM.

Preferred methods include embodiments wherein an immune modulatory agent is administered to the combination of immune cells and regenerative cells.

Preferred methods include embodiments wherein said immune modulatory agent is curcumin.

Preferred methods include embodiments wherein said immune modulatory agent is TGF-beta.

Preferred methods include embodiments wherein said immune modulatory agent is galectin-1.

Preferred methods include embodiments wherein said immune modulatory agent is galectin-3.

Preferred methods include embodiments wherein said immune modulatory agent is galectin-9.

Preferred methods include embodiments wherein said immune modulatory agent is IL-1.

Preferred methods include embodiments wherein said immune modulatory agent is IL-2.

Preferred methods include embodiments wherein said immune modulatory agent is IL-4.

Preferred methods include embodiments wherein said immune modulatory agent is IL-7.

Preferred methods include embodiments wherein said immune modulatory agent is IL-10.

Preferred methods include embodiments wherein said immune modulatory agent is IL-13.

Preferred methods include embodiments wherein said immune modulatory agent is IL-15.

Preferred methods include embodiments wherein said immune modulatory agent is IL-12.

Preferred methods include embodiments wherein said immune modulatory agent is IL-18.

Preferred methods include embodiments wherein said immune modulatory agent is IL-20.

Preferred methods include embodiments wherein said immune modulatory agent is IL-22.

Preferred methods include embodiments wherein said immune modulatory agent is IL-35.

Preferred embodiments include method of reducing renal damage after an ischemia/reperfusion injury insult, said method comprising of; a) obtaining a patient who has underwent a renal ischemia/reperfusion injury; b) extracting from said patient monocytes; c) culturing monocytes from said patient with a regenerative cell; d) priming said monocytes with renal derived antigens; e) generating a population of tolerogeneic dendritic cells from said monocytes, wherein said tolerogenic dendritic cells are pulsed with renal derived antigens and f) administering said cells into said patient who has underwent renal ischemia/reperfusion injury.

Preferred methods include embodiments wherein said renal ischemia/reperfusion injury is associated with release of renal antigens.

Preferred embodiments include methods of preventing or treating kidney disease in an individual having, or at risk of having, kidney disease comprising administering a prophylactically or therapeutically effective amount of immune system cells that have been exposed to regenerative cells.

Preferred methods include embodiments wherein the regenerative cells are derived from tissue selected from the group consisting of skin, adipose tissue, bone marrow, umbilical cord, Wharton's jelly, omentum, peripheral blood, mobilized peripheral blood, and a combination thereof.

Preferred methods include embodiments wherein the mobilized peripheral blood is obtained by administration of agents selected from the group consisting of G-CSF, GM-CSF, flt-3 ligand, plerixafor (including Mozobil™), hyperbaric oxygen, ozone therapy, and a combination thereof.

Preferred methods include embodiments wherein said regenerative cells express markers selected from the group consisting of extracellular vimentin, Cyclin D2, Snail, E-cadherin, SOX-2, CD105, CD90, CD29, CD73, Wt1, and a combination thereof.

Preferred methods include embodiments wherein said regenerative cells are autologous with respect to the individual.

Preferred methods include embodiments wherein said regenerative cells are allogeneic with respect to the individual.

Preferred methods include embodiments wherein said regenerative cells are treated with one or more compositions capable of enhancing ability to suppress production of TGF-beta from Th3 cells.

Preferred methods include embodiments wherein the composition capable of enhancing ability to suppress production of TGF-beta from Th3 cells comprises oxytocin.

Preferred methods include embodiments wherein oxytocin is exposed to the regenerative cells in vitro for a period ranging between 1 minute to 4 weeks.

Preferred methods include embodiments wherein oxytocin is exposed to the regenerative cells in vitro for a period ranging between 2 hours to 1 week.

Preferred methods include embodiments wherein oxytocin is exposed to the regenerative cells in vitro for a period ranging between 24 hours to 72 hours.

Preferred methods include embodiments wherein oxytocin is administered at a concentration ranging between 10 nM-10 μM.

Preferred methods include embodiments wherein said oxytocin is administered at a concentration ranging between 100 nM-1 μM.

Preferred methods include embodiments wherein said regenerative cells are assessed for an ability to inhibit production of TGF-beta from Th3 cells subsequent to cell to cell contact between said regenerative cells and said Th3 cells.

Preferred methods include embodiments wherein the kidney disease is associated with chemotherapy administration.

Preferred methods include embodiments wherein the kidney disease is associated with radiation administration.

Preferred methods include embodiments wherein said kidney disease is quantified as an increase in serum creatinine level of at least 0.5 mg/dL over a baseline serum creatinine measured in the individual.

Preferred methods include embodiments wherein said kidney disease is quantified as an increase in serum creatinine level of between 0.3 mg/dL and 0.5 mg/dL over a baseline serum creatinine measured in the individual.

Preferred methods include embodiments wherein said kidney disease is quantified as a serum creatinine level higher than 1.0 measured in the individual.

Preferred methods include embodiments wherein said kidney disease is quantified by an increase in one or more serum/blood biomarkers, one or more urine biomarkers, or both.

Preferred methods include embodiments wherein the one or more serum/blood biomarkers are selected from the group consisting of blood urea nitrogen (BUN), cystatin C, beta-trace protein (BTP), and a combination thereof.

Preferred methods include embodiments wherein the one or more urine biomarkers are selected from the group consisting of podocalyxin, nephrin, alpha 1-microglobulin, beta 2-microglobulin, glutathione S-transferase, interleukin-18, kidney injury molecule-1 (KIM-1), liver-type fatty acid-binding protein, netrin-1, neutrophil gelatinase-associated lipocalcin (NGAL), and n-acetyl-beta-d-glucosaminidase (NAG), and a combination thereof.

Preferred methods include embodiments wherein the kidney disease is further assessed by an increase in one or more biomarkers selected from the group consisting of blood urea nitrogen (BUN), cystatin c, beta-trace protein (BTP), podocalyxin, nephrin, alpha 1-microglobulin, beta 2-microglobulin, glutathione s-transferase, interleukin-18, kidney injury molecule-1 (KIM-1), liver-type fatty acid-binding protein, netrin-1, neutrophil gelatinase-associated lipocalcin (NGAL), n-acetyl-beta-d-glucosaminidase (NAG), and a combination thereof.

Preferred methods include embodiments wherein the prophylactically or therapeutically effective amount of regenerative cells is between about approximately 7×105 cells/kg of individual's body weight and approximately 7×106 cells/kg of individual's body weight.

Preferred methods include embodiments wherein the prophylactically or therapeutically effective amount of regenerative cells is between about approximately 2×106 cells/kg of individual's body weight and approximately 5×106 cells/kg of individual's body weight.

Preferred methods include embodiments wherein the prophylactically or therapeutically effective amount of regenerative cells is approximately 2×106 cells/kg of individual's body weight.

Preferred methods include embodiments wherein the regenerative cells are administered to the individual at the onset or diagnosis of the kidney disease.

Preferred methods include embodiments wherein the regenerative cells are administered to the individual at least 24 hours following the onset or diagnosis of the kidney disease.

Preferred methods include embodiments wherein the regenerative cells are administered to the individual at least 48 hours following the onset or diagnosis of the kidney disease.

Preferred methods include embodiments wherein the regenerative cells are administered to the individual between onset or diagnosis of the kidney disease and 24 hours following the onset or diagnosis of the kidney disease.

Preferred methods include embodiments wherein the regenerative cells are administered to the individual between 24 and 48 hours following the onset or diagnosis of the kidney disease.

Preferred methods include embodiments wherein the regenerative cells are administered intra-arterially or intravenously to the individual.

### BRIEF DESCRIPTION OF THE DRAWINGS

FIG. 1 is a bar graph showing Creatinine levels based on various mice and treatments including: control, PBS+Clamp, MSCs+Clamp, and ImmCelz+Clamp.

### DESCRIPTION OF THE INVENTION

The present disclosure is directed to system, methods, and compositions for preventing or treating an individual having or at risk of having kidney disease. Certain embodiments of the disclosure concern preventing or treating the loss of or reduction of kidney function, including resulting from one or more diseases and/or nephrotoxic compositions. The disease causing a loss of function in the kidney may be immune-mediated, infection-mediated, metabolism-mediated, hormone-mediated, genetically-mediated, or mediated by any other biological process. In some embodiments, the disclosure concerns treatment or prevention for exposure to one or more nephrotoxic compositions and/or agents to an individual, whether intentionally or accidentally. The nephrotoxic compositions and/or agents may include, but are not limited to, chemotherapies, radiation, immune-modulating compositions or agents, non-steroid anti-inflammatory drugs (NSAIDs), antibiotics, antifungals, antivirals, diuretics, beta blockers, ACE inhibitors, vasodilators, cyclosporins, steroids, narcotics, combinations thereof, or any other composition and/or agent that may induce nephrotoxicity.

In certain embodiments, the loss of kidney function may comprise a change in the level of one or more serum and/or blood markers including, but not limited to, creatinine, blood urea nitrogen (BUN), cystatin C, beta-trace protein, podocalyxin, nephrin, alpha 1-microglobulin, beta 2-microglobulin, glutathione S-transferase, interleukin-18, kidney injury molecule-1 (KIM-1), liver-type fatty acid-binding protein, netrin-1, liver-type fatty acid-binding protein, neutrophil gelatinase-associated lipocalcin (NGAL), n-acetyl-beta-d-glucosaminidase (NAG), or a combination thereof. The change in level may or may not be assessed prior to and/or after treatment of loss of kidney function in the individual.

In some embodiments, the disease and/or exposure to one or more nephrotoxic compositions causes an increase in the serum creatinine in an individual, and the level of serum creatinine may or may not be determined in the individual prior to and/or after treatment of loss of kidney function in the individual. In some cases, the increase in serum creatinine may be more than 0.5 mg/dL over a baseline measurement performed on the individual. The increase in serum creatinine may be an increase between 0.3 mg/dL to 0.5 mg/dL over a baseline measurement performed on the individual. In some embodiments, the serum creatinine levels in an individual having, or at risk of having, kidney disease may be more than 1.0, 1.1, 1.2, 1.3, 1.4, 1.5, 1.6, 1.7, 1.8, 1.9, 2.0, 2.1, 2.2, 2.3, 2.4, 2.5, 3.0, 4.0, 5.0 mg/dL or higher.

Certain embodiments of the present disclosure concern the administration of an effective amount of immune cells that have been previously exposed to regenerative cells, to an individual having, or at risk of having, kidney disease, including any kidney disease or partial or full loss of kidney function described herein. The reprogrammed immune cells, which have been reprogrammed by exposure to stem cells, otherwise known as regenerative cells, may be administered in any suitable manner, including intra-arterially and/or intravenously, merely as examples. In some embodiments, the reprogrammed immune cells are administered to an individual of the present disclosure therapeutically and/or prophylactically. The immune cells may be administered to the individual at the onset or initial diagnosis of the kidney disease. The immune cells may be administered less than about 24 hours or less than about 48 hours after the onset or diagnosis of the kidney disease. The immune cells may be administered more than about 24 hours or more than about 48 hours after the onset or diagnosis of the kidney disease. The immune cells may be administered between about 24 to about 48 hours after the onset or diagnosis of the kidney disease. The immune cells may be administered approximately 24, 26, 28, 30, 32, 34, 36, 38, 40, 42, 44, 46, or 48 hours after the onset or diagnosis of the kidney disease.

In some embodiments, an individual receiving methods and compositions of the present disclosure is administered a prophylactically or therapeutically effective amount of immune cells of the present disclosure. The prophylactically or therapeutically effective amount of immune cells may be between approximately 7×105 cells/kg of individual's body weight and approximately 7×106 cells/kg of individual's body weight. In some embodiments, the prophylactically or therapeutically effective amount of reprogrammed immune cells is between about approximately 2×106 cells/kg of individual's body weight and approximately 5×106 cells/kg of individual's body weight. In some embodiments, the prophylactically or therapeutically effective amount of reprogrammed immune cells approximately 2×106 cells/kg of individual's body weight.

In some embodiments, the reprogrammed immune cells of the present disclosure are from an autologous source with respect to an individual subjected to the methods of the present disclosure. In some embodiments, the reprogrammed immune cells of the present disclosure are from an allogeneic source with respect to an individual subjected to methods of the present disclosure. The reprogrammed immune cells of the present disclosure may be from any source or tissue including reprogrammed immune cells derived from at least skin, adipose tissue, bone marrow, umbilical cord, Wharton's jelly, omentum, peripheral blood, mobilized peripheral blood, or a combination thereof. In some embodiments, the reprogrammed immune cells express one or more markers including, but not limited to, extracellular vimentin, cyclin D2, Snail, E-cadherin, SOX-2, CD105, CD90, CD29, CD73, WT1, or a combination thereof.

In some embodiments, the reprogrammed immune cells of the present disclosure are modified, such as by exposure to one or more compositions, to enhance the ability of the reprogrammed immune cells to suppress the production of TGF-beta in Th3 cells. The modification of the reprogrammed immune cells may be monitored by assessing the inhibited production of TGF-beta from Th3 cells subsequent to cell-to-cell contact between modified reprogrammed immune cells and Th3 cells. Thus, reprogrammed immune cells may be modified upon exposure to one or more agents and/or conditions, followed by delivery of the reprogrammed immune cells to an individual in need thereof, upon which Th3 cells in the individual subsequently have reduced production of TGF-beta.

A composition for modifying reprogrammed immune cells may comprise oxytocin. In some embodiments, the reprogrammed immune cells are exposed to a composition, such as oxytocin, for a period between approximately 1 minute to approximately 4 weeks, or between approximately 2 hours to approximately 1 week, or between approximately 24 hours to approximately 72 hours. The reprogrammed immune cells may be exposed to oxytocin at a concentration ranging between approximately 10 nM to approximately 10 μM, or between approximately 100 nM to approximately 1 μM. The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description.

“Allogeneic,” as used herein, refers to cells of the same species that differ genetically from cells of a host.

“Autologous,” as used herein, refers to cells derived from the same subject. The term “engraft” as used herein refers to the process of stem cell incorporation into a tissue of interest in vivo through contact with existing cells of the tissue.

“Approximately” or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Carrier” or diluent: As used herein, the terms “carrier” and “diluent” refers to a pharmaceutically acceptable (e.g., safe and non-toxic for administration to a human) carrier or diluting substance useful for the preparation of a pharmaceutical formulation. Exemplary diluents include sterile water, bacteriostatic water for injection (BWFI), a pH buffered solution (e.g. phosphate-buffered saline), sterile saline solution, Ringer's solution or dextrose solution.

As used herein, the terms “dosage form” and “unit dosage form” refer to a physically discrete unit of a therapeutic agent for the individual to be treated. Each unit contains a predetermined quantity of active material calculated to produce the desired therapeutic effect. It will be understood, however, that the total dosage of the composition will be decided by the attending physician within the scope of sound medical judgment.

A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, the therapeutic agent is administered continuously over a predetermined period. In some embodiments, the therapeutic agent is administered once a day (QD) or twice a day (BID).

By “does not detectably express” means that expression of a protein or gene cannot be detected by standard methods. In the case of cell surface markers, expression can be measured by, e.g., flow cytometry, using a cut-off values as obtained from negative controls (i.e., cells known to lack the antigen of interest) or by isotype controls (i.e., measuring nonspecific binding of the antibody to the cell). Thus, a cell that “does not detectably express” a marker appears similar to the negative control for that marker. For gene expression, a gene “does not detectably express” if the presence of its mRNA cannot be visually detected on a standard agarose gel following standard PCR protocols. Conversely, a cell “expresses” the protein or gene if it can be detected by the same method.

The term “culture expanded population” means a population of cells whose numbers have been increased by cell division in vitro. This term may apply to stem cell populations and non-stem cell populations alike.

The term “passaging” refers to the process of transferring a portion of cells from one culture vessel into a new culture vessel.

The term “cryopreserve” refers to preserving cells for long term storage in a cryoprotectant at low temperature.

The term “master cell bank” refers to a collection of cryopreserved cells. Such a cell bank may comprise stem cells, non-stem cells, and/or a mixture of stem cells and non-stem cells.

The invention teaches the generation of reprogrammed immune cells that are useful for prevention of kidney disease.

General principles of maintaining adherent cell cultures are well-known in the art. As appreciated by those skilled in the art, the fibroblast cells may be counted in order to facilitate subsequent plating of the cells at a desired density. Where, as in the present disclosure, the cells after plating may primarily adhere to a substrate surface present in the culture system (e.g., in a culture vessel), the plating density may be expressed as number of cells plated per mm2 or cm2 of the said substrate surface. In practicing the disclosure, after plating of the regenerative cells, the cell suspension is left in contact with the adherent surface to allow for adherence of cells from the cell population to the substrate. In contacting regenerative cells to the adherent substrate, the cells may be advantageously suspended in an environment comprising at least a medium, in the methods of the disclosure typically a liquid medium, which supports the survival and/or growth of the cells. The medium may be added to the system before, together with or after the introduction of the cells thereto. The medium may be fresh, i.e., not previously used for culturing of cells, or may comprise at least a portion which has been conditioned by prior culturing of cells therein, e.g., culturing of the cells which are being plated or antecedents thereof, or culturing of cells more distantly related to or unrelated to the cells being plated.

The medium may be a suitable culture medium as described elsewhere in this specification. Preferably, the composition of the medium may have the same features, may be the same or substantially the same as the composition of medium used in the ensuing steps of culturing the attached cells. Otherwise, the medium may be different. In some embodiments, the cells from the fibroblast cell population or from tissue explants of the present disclosure, which have adhered to the substrate, preferably in the environment, are subsequently cultured for at least 7 days, for at least 8 days, or for at least 9 days, for at least 10 days, at least 11, or at least 12 days, at least 13 days or at least 14 days, for at least 15 days, for at least 16 days or for at least 17 days, or even for at least 18 days, for at least 19 days or at least 21 days or more. The term “culturing” is common in the art and broadly refers to maintenance and/or growth of cells and/or progeny thereof.

In some embodiments, the fibroblast cells may be cultured for at least between about 10 days and about 40 days, for at least between about 15 days and about 35 days, for at least between about 15 days and 21 days, such as for at least about 15, 16, 17, 18, 19 or 21 days. In some embodiments, the stem cells of the disclosure may be cultured for no longer than 60 days, or no longer than 50 days, or no longer than 45 days. The tissue explants and regenerative cells may be cultured in the presence of a liquid culture medium. Typically, the medium will comprise a basal medium formulation as known in the art. Many basal media formulations can be used to culture regenerative cells herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, and modifications and/or combinations thereof. Compositions of the above basal media are generally known in the art and it is within the skill of one in the art to modify or modulate concentrations of media and/or media supplements as necessary for the regenerative cells cultured. In some embodiments, a culture medium formulation may be explants medium (CEM) which is composed of IMDM supplemented with 10% fetal bovine serum (FBS, Lonza), 100 U/ml penicillin G, 100 .mu.g/ml streptomycin and 2 mmol/L L-glutamine (Sigma-Aldrich). Other embodiments may employ further basal media formulations, such as chosen from the ones above.

For use in the fibroblast culture, media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks' Balanced Salt Solution (HBSS), Earle's Salt Solution. Further antioxidant supplements may be added, e.g., beta-mercaptoethanol. While many media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin. Also contemplated is supplementation of cell culture medium with mammalian plasma or sera. Plasma or sera often contain cellular factors and components that are necessary for viability and expansion. The use of suitable serum replacements is also contemplated (e.g., FBS).

In some embodiments, the regenerative cells of the present disclosure are modified, such as by exposure to a composition, to enhance the ability of the regenerative cells to suppress the production of TGF-beta in Th3 cells. The modification of the regenerative cells may be monitored by assessing the inhibited production of TGF-beta from Th3 cells subsequent to cell to cell contact between modified regenerative cells and Th3 cells. The composition for modifying regenerative cells may comprise oxytocin, in specific cases. In some embodiments, the regenerative cells are exposed to the composition, such as oxytocin, for a period between approximately 1 minute to approximately 4 weeks, or between approximately 2 hours to approximately 1 week, or between approximately 24 hours to approximately 72 hours. The regenerative cells may be exposed to oxytocin at a concentration ranging between approximately 10 nM to approximately 10 μM, or between approximately 100 nM to approximately 1 μM.

As described, the present inventors have identified that by culturing tissue explants and fibroblast cells for time durations as defined above, and in at least some cases using media compositions as described above, a progenitor or stem cell of the disclosure emerges and proliferates. In some embodiments, fibroblast cells of the present disclosure are identified and characterized by their expression of specific marker proteins, such as cell-surface markers. Detection and isolation of these cells can be achieved, e.g., through flow cytometry, ELISA, and/or magnetic beads. Reverse-transcription polymerase chain reaction (RT-PCR) can also be used to monitor changes in gene expression in response to differentiation. Methods for characterizing regenerative cells the present disclosure are provided herein. In certain embodiments, the marker proteins used to identify and characterize the stem cells are selected from the list consisting of c-Kit, Nanog, Sox2, Hey1, SMA, Vimentin (including extracellular vimentin), Cyclin D2, Snail, E-cadherin, Nkx2.5, GATA4, CD105, CD90, CD29, CD73, Wt1, CD34, CD45, and a combination thereof.

In one embodiment the invention teaches phenotypically defined MSC which can be isolated from the Wharton's jelly of umbilical cord segments and defined morphologically and by cell surface markers. By dissecting out the veins and arteries of cord segments and exposing the Wharton's jelly, the cells of invention, of one embodiment of the invention, may be obtained. An approximately 1-5 cm cord segment is placed in collagenase solution (1 mg/ml, Sigma) for approximately 18 hrs at room temperature. After incubation, the remaining tissue is removed and the cell suspension is diluted with PBS into two 50 ml tubes and centrifuged. Cells are then washed in PBS and counted using hematocytometer. 5-20.times.10.sup.6 cells were then plated in a 6 cm tissue culture plate in low-glucose DMEM (Gibco) with 10% FBS (Hyclone), 2 mM L-Glutamine (Gibco), 100 U/ml penicillin/100 ug/ml streptomycin/0.025 ug/ml amphotericin B (Gibco). At this step of the purification process, cells are exposed to hypoxia. The amount of hypoxia needed is the sufficient amount to induce activagion of HIF-1 alpha. In one embodiment cells are cultured for 24 hours at 2% oxygen. After 48 hrs cells are washed with PBS and given fresh media. Cells were given new media twice weekly. After 7 days, cells are approximately 70-80% confluent and are passed using HyQTase (Hyclone) into a 10 cm plate. Cells are then regularly passed 1:2 every 7 days or upon reaching 80% confluence.

In another embodiment of the invention, biologically useful stem cells are disclosed, of the mesenchymal or related lineages, which are therapeutically reprogrammed cells having minimal oxidative damage and telomere lengths that compare favorably with the telomere lengths of undamaged, pre-natal or embryonic stem cells (that is, the therapeutically reprogrammed cells of the present invention possess near prime physiological state genomes). Moreover the therapeutically reprogrammed cells of the present invention are immunologically privileged and therefore suitable for therapeutic applications. Additional methods of the present invention provide for the generation of hybrid stem cells. Furthermore, the present invention includes related methods for maturing stem cells made in accordance with the teachings of the present invention into specific host tissues. For use in the current invention, the practitioner is thought that ontogeny of mammalian development provides a central role for stem cells. Early in embryogenesis, cells from the proximal epiblast destined to become germ cells (primordial germ cells) migrate along the genital ridge. These cells express high levels of alkaline phosphatase as well as expressing the transcription factor Oct4. Upon migration and colonization of the genital ridge, the primordial germ cells undergo differentiation into male or female germ cell precursors (primordial sex cells). For the purpose of this invention disclosure, only male primordial sex cells (PSC) will be discussed, but the qualities and properties of male and female primordial sex cells are equivalent and no limitations are implied. During male primordial sex cell development, the primordial stem cells become closely associated with precursor sertoli cells leading to the beginning of the formation of the seminiferous cords. When the primordial germ cells are enclosed in the seminiferous cords, they differentiate into gonocytes that are mitotically quiescent. These gonocytes divide for a few days followed by arrest at G0/G1 phase of the cell cycle. In mice and rats these gonocytes resume division within a few days after birth to generate spermatogonial stem cells and eventually undergo differentiation and meiosis related to spermatogenesis. It is known that embryonic stem cells are cells derived from the inner cell mass of the pre-implantation blastocyst-stage embryo and have the greatest differentiation potential, being capable of giving rise to cells found in all three germ layers of the embryo proper. From a practical standpoint, embryonic stem cells are an artifact of cell culture since, in their natural epiblast environment, they only exist transiently during embryogenesis. Manipulation of embryonic stem cells in vitro has lead to the generation and differentiation of a wide range of cell types, including cardiomyocytes, hematopoietic cells, endothelial cells, nerves, skeletal muscle, chondrocytes, adipocytes, liver and pancreatic islets. Growing embryonic stem cells in co-culture with mature cells can influence and initiate the differentiation of the embryonic stem cells to a particular lineage. Maturation is a process of coordinated steps either forward or backward in the differentiation pathway and can refer to both differentiation and/or dedifferentiation. In one example of the maturation process, a cell, or group of cells, interacts with its cellular environment during embryogenesis and organogenesis. As maturation progresses, cells begin to form niches and these niches, or microenvironments, house stem cells that direct and regulate organogenesis. At the time of birth, maturation has progressed such that cells and appropriate cellular niches are present for the organism to function and survive post-natally. Developmental processes are highly conserved amongst the different species allowing maturation or differentiation systems from one mammalian species to be extended to other mammalian species in the laboratory. During the lifetime of an organism, the cellular composition of the organs and organs systems are exposed to a wide range of intrinsic and extrinsic factors that induce cellular or genomic damage. Ultraviolet light not only has an effect on normal skin cells but also on the skin stem cell population. Chemotherapeutic drugs used to treat cancer have a devastating effect on hematopoietic stem cells. Reactive oxygen species, which are the byproducts of cellular metabolism, are intrinsic factors that compromises the genomic integrity of the cell. In all organs or organ systems, cells are continuously being replaced from stem cell populations. However, as an organism ages, cellular damage accumulates in these stem cell populations. If the damage is inheritable, such as genomic mutations, then all progeny will be effected and thus compromised. A single stem cell clone can contribute to generations of lineages such as lymphoid and myeloid cells for more than a year and therefore have the potential to spread mutations if the stem cell is damaged. The body responds to a compromised stem cell by inducing apoptosis thereby removing it from the pool and preventing potentially dysfunctional or tumorigenic properties. Apoptosis removes compromised cells from the population, but it also decreases the number of stem cells that are available for the future. Therefore, as an organism ages, the number of stem cells decrease. In addition to the loss of the stem cell pool, there is evidence that aging decreases the efficiency of the homing mechanism of stem cells. Telomeres are the physical ends of chromosomes that contain highly conserved, tandemly repeated DNA sequences. Telomeres are involved in the replication and stability of linear DNA molecules and serve as counting mechanism in cells; with each round of cell division the length of the telomeres shortens and at a pre-determined threshold, a signal is activated to initiate cellular senescence. Stem cells and somatic cells produce telomerase, which inhibits shortening of telomeres, but their telomeres still progressively shorten during aging and cellular stress. In one teaching, or embodiment, of the invention, therapeutically reprogrammed cells, in some embodiments mesenchymal stem cells, are provided. Therapeutic reprogramming refers to a maturation process wherein a stem cell is exposed to stimulatory factors according the teachings of the present invention to yield enhanced therapeutic activity. In some embodiments, enhancement of therapeutic activity may be increase proliferation, in other embodiments, it may be enhanced chemotaxis. Other therapeutic characteristics include ability to under resistance to apoptosis, ability to overcome senescence, ability to differentiate into a variety of different cell types effectively, and ability to secrete therapeutic growth factors which enhance viability/activity, of endogenous stem cells. In order to induce therapeutic reprogramming of cells, in some cases, as disclosed herein, of wharton's jelly originating cells, the invention teaches the utilization of stimulatory factors, including without limitation, chemicals, biochemicals and cellular extracts to change the epigenetic programming of cells. These stimulatory factors induce, among other results, genomic methylation changes in the donor DNA. Embodiments of the present invention include methods for preparing cellular extracts from whole cells, cytoplasts, and karyplasts, although other types of cellular extracts are contemplated as being within the scope of the present invention. In a non-limiting example, the cellular extracts of the present invention are prepared from stem cells, specifically embryonic stem cells. Donor cells are incubated with the chemicals, biochemicals or cellular extracts for defined periods of time, in a non-limiting example for approximately one hour to approximately two hours, and those reprogrammed cells that express embryonic stem cell markers, such as Oct4, after a culture period are then ready for transplantation, cryopreservation or further maturation. In another embodiment of the present invention, hybrid stem cells are provided which can be used for cellular regenerative/reparative therapy. The hybrid stem cells of the present invention are pluripotent and customized for the intended recipient so that they are immunologically compatible with the recipient. Hybrid stem cells are a fusion product between a donor cell, or nucleus thereof, and a host cell. Typically the fusion occurs between a donor nucleus and an enucleated host cell. The donor cell can be any diploid cell, including but not limited to, cells from pre-embryos, embryos, fetuses and post-natal organisms. More specifically, the donor cell can be a primordial sex cell, including but not limited to, oogonium or differentiated or undifferentiated spermatogonium, or an embryonic stem cell. Other non-limiting examples of donor cells are therapeutically reprogrammed cells, embryonic stem cells, fetal stem cells and multipotent adult progenitor cells. Preferably the donor cell has the phenotype of the intended recipient. The host cell can be isolated from tissues including, but not limited to, pre-embryos, embryos, fetuses and post-natal organisms and more specifically can include, but is not limited to, embryonic stem cells, fetal stem cells, multipotent adult progenitor cells and adipose-derived stem cells. In a non-limiting example, cultured cell lines can be used as donor cells. The donor and host cells can be from the same individual or different individuals. In one embodiment of the present invention, lymphocytes are used as donor cells and a two-step method is used to purify the donor cells. After the tissues was disassociated, an adhesion step was performed to remove any possible contaminating adherent cells followed by a density gradient purification step. The majority of lymphocytes are quiescent (in G0 phase) and therefore can have a methylation status than conveys greater plasticity for reprogramming. Multipotent or pluripotent stem cells or cell lines useful as donor cells in embodiments of the present invention are functionally defined as stem cells by their ability to undergo differentiation into a variety of cell types including, but not limited to, adipogenic, neurogenic, osteogenic, chondrogenic and cardiogenic cell.

In some embodiments, host cell enucleation for the generation of hybrid stem cells according to the teachings of the present invention can be conducted using a variety of means. In a non-limiting example, ADSCs were plated onto fibronectin coated tissue culture slides and treated with cells with either cytochalasin D or cytochalasin B. After treatment, the cells can be trypsinized, re-plated and are viable for about 72 hours post enucleation. Host cells and donor nuclei can be fused using one of a number of fusion methods known to those of skill in the art, including but not limited to electrofusion, microinjection, chemical fusion or virus-based fusion, and all methods of cellular fusion are envisioned as being within the scope of the present invention. The hybrid stem cells made according to the teachings of the present invention possess surface antigens and receptors from the enucleated host cell but has a nucleus from a developmentally younger cell. Consequently, the hybrid stem cells of the present invention will be receptive to cytokines, chemokines and other cell signaling agents, yet possess a nucleus free from age-related DNA damage. The therapeutically reprogrammed cells and hybrid stem cells made in accordance with the teachings of the present invention are useful in a wide range of therapeutic applications for cellular regenerative/reparative therapy. For example, and not intended as a limitation, the therapeutically reprogrammed cells and hybrid stem cells of the present invention can be used to replenish stem cells in animals whose natural stem cells have been depleted due to age or ablation therapy such as cancer radiotherapy and chemotherapy. In another non-limiting example, the therapeutically reprogrammed cells and hybrid stem cells of the present invention are useful in organ regeneration and tissue repair. In one embodiment of the present invention, therapeutically reprogrammed cells and hybrid stem cells can be used to reinvigorate damaged muscle tissue including dystrophic muscles and muscles damaged by ischemic events such as myocardial infarcts. In another embodiment of the present invention, the therapeutically reprogrammed cells and hybrid stem cells disclosed herein can be used to ameliorate scarring in animals, including humans, following a traumatic injury or surgery. In this embodiment, the therapeutically reprogrammed cells and hybrid stem cells of the present invention are administered systemically, such as intravenously, and migrate to the site of the freshly traumatized tissue recruited by circulating cytokines secreted by the damaged cells. In another embodiment of the present invention, the therapeutically reprogrammed cells and hybrid stem cells can be administered locally to a treatment site in need or repair or regeneration.

In one embodiment, umbilical cord samples were obtained following the delivery of normal term babies with Institutional Review Board approval. A portion of the umbilical cord was then cut into approximately 3 cm long segments. The segments were then placed immediately into 25 ml of phosphate buffered saline without calcium and magnesium (PBS) and 1.times. antibiotics (100 U/ml penicillin, 100 ug/ml streptomycin, 0.025 ug/ml amphotericin B). The tubes were then brought to the lab for dissection within 6 hours. Each 3 cm umbilical cord segment was dissected longitudinally utilizing aseptic technique. The tissue was carefully undermined and the umbilical vein and both umbilical arteries were removed. The remaining segment was sutured inside out and incubated in 25 ml of PBS, 1.times. antibiotic, and 1 mg/ml of collagenase at room temperature. After 16-18 hours the remaining suture and connective tissue was removed and discarded. The cell suspension was separated equally into two tubes, the cells were washed 3.times. by diluting with PBS to yield a final volume of 50 ml per tube, and then centrifuged. Red blood cells were then lysed using a hypotonic solution. Cells were plated onto 6-well plates at a concentration of 5-20.times.10.sup.6 cells per well. UC-MSC were cultured in low-glucose DMEM (Gibco) with 10% FBS (Hyclone), 2 mM L-Glutamine (Gibco), 100 U/ml penicillin, 100 ug/ml streptomycin, 0.025 ug/ml amphotericin B (Gibco). Cells were washed 48 hours after the initial plating with PBS and given fresh media. Cell culture media were subsequently changed twice a week through half media changes. After 7 days or approximately 70-80% confluence, cells were passed using HyQTase (Hyclone) into a 10 cm plate. Cells were then regularly passed 1:2 every 7 days or upon reaching 80% confluence. Alternatively, 0.25% HQ trypsin/EDTA (Hyclone) was used to passage cells in a similar manner.

In some embodiments of the invention, administration of cells of the invention is performed for suppression of an inflammatory and/or autoimmune disease. In these situations, it may be necessary to utilize an immune suppressive/or therapeutic adjuvant. Immune suppressants are known in the art and can be selected from a group comprising of: cyclosporine, rapamycin, campath-1H, ATG, Prograf, anti IL-2r, MMF, FTY, LEA, cyclosporin A, diftitox, denileukin, levamisole, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, and trimetrexate), purine analogs (e.g., cladribine, fludarabine, 6-mercaptopurine, thiamiprine, and thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, and tegafur) fluocinolone, triaminolone, anecortave acetate, fluorometholone, medrysone, prednislone, etc. In another embodiment, the use of stem cell conditioned media may be used to potentiate an existing anti-inflammatory agent. Anti-inflammatory agents may comprise one or more agents including NSAIDs, interleukin-1 antagonists, dihydroorotate synthase inhibitors, p38 MAP kinase inhibitors, TNF-α inhibitors, TNF-α sequestration agents, and methotrexate. More specifically, anti-inflammatory agents may comprise one or more of, e.g., anti-TNF-α, lysophylline, alpha 1-antitrypsin (AAT), interleukin-10 (IL-10), pentoxyfilline, COX-2 inhibitors, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (eg., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), epsilon.-acetamidocaproic acid, s-adenosylmethionine, 3-amino-4-hydroxybutyric acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, candelilla wax, alpha bisabolol, aloe vera, Manjistha, Guggal, kola extract, chamomile, sea whip extract, glycyrrhetic acid, glycyrrhizic acid, oil soluble licorice extract, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid.

The introduction of “biological therapies” such as anti-TNF-alpha antibodies has led to some improvements in prognosis, although side effects are still present due to the non-specific nature of the intervention. The same holds true for cytokine storm conditions such as sepsis, where overproduction of agents such as TNF-alpha result in vascular leakage, coagulopathy, and death. The invention provides the utilization of tolerance-induction using regenerative cells, in one embodiment, said regenerative cells are capable of inducing infectious tolerance alone, or in combination with existing techniques. The utilization of antigen-nonspecific immature dendritic cells which are generated by co-culture with regenerative cells allows for induction of a inhibitory immune response, which results in suppression of autoimmune or alloimmune inflammation. The invention teaches that to effectively treat conditions of immune overactivation it is important to delete/inactivate the T cell clone that are associated with stimulation of inflammation, as well as to block innate immune elements. This would be akin to recapitulating the natural process of tolerance induction. While thymic deletion was the original process identified as being responsible for selectively deleting autoreactive T cells, it became clear that numerous redundant mechanisms exist that are not limited to the neonatal period. Specifically, a “mirror image” immune system was demonstrated to co-exist with the conventional immune system. Conventional T cells are activated by self-antigens to die in the thymus and conventional T cells that are not activated receive a survival signal [1]; the “mirror image”, T regulatory (Treg) cells are actually selected to live by encounter with self-antigens, and Treg cells that do not bind self antigens are deleted [2, 3]. In one embodiment of the invention, immature dendritic cells are administered in order to induce a state of immune modulation, including T regulatory cell generation by the immature dendritic cells. Utilization of immature dendritic cells to stimulate T regulatory cell proliferation and/or activity has been previously demonstrated and is incorporated by reference [4-10].

Thus the self-nonself discrimination by the immune system occurs in part based on self antigens depleting autoreactive T cells, while promoting the generation of Treg cells. An important point for development of an antigen-specific tolerogenic vaccine is that in adult life, and in the periphery, autoreactive T cells are “anergized” by presentation of self-antigens in absence of danger signals, and autoreactive Treg are generated in response to self antigens. Although the process of T cell deletion in the thymus is different than induction of T cell anergy, and Treg generation in the thymus, results in a different type of Treg as compared to peripheral induced Treg, in many aspects, the end result of adult tolerogenesis is similar to that which occurs in the neonatal period.

Specific examples of tolerogenesis that occurs in adults includes settings such as pregnancy, cancer, and oral tolerance. In the situation of pregnancy, studies have demonstrated selective inactivation of maternal T cell clones that recognize fetal antigens occurs through a variety of mechanisms, including FasL expression on fetal and placental cells [11], antigen presentation in the context of PD1-L [12], and HLA-G interacting with immune inhibitory receptors such as ILT4 [13]. In pregnancy, “tolerogenic antigen presentation” occurs only through the indirect pathway of antigen presentation [14]. Other pathways of selective tolerogenesis in pregnancy include the stimulation of Treg cells, which have been demonstrated essential for successful pregnancy [15]. In the context of cancer, depletion of tumor specific T cells, while sparing of T cells with specificities to other antigens has been demonstrated by the tumor itself or tumor associated cells [16-19]. Additionally, Treg cells have been demonstrated to actively suppress anti-tumor T cells, perhaps as a “back up” mechanism of tumor immune evasion [20-22]. At a clinical level the ability of tumors to inhibit peripheral T cell activity has been associated in numerous studies with poor prognosis [23-25]. Oral tolerance is the process by which ingested antigens induce generation of antigen-specific TGF-beta producing cells (called “Th3” by some) [26-28], as well as Treg cells [29, 30]. Ingestion of antigen, including the autoantigen collagen II [31], has been shown to induce inhibition of both T and B cell responses in a specific manner [32, 33]. It appears that induction of regulatory cells, as well as deletion/anergy of effector cells is associated with antigen presentation in a tolerogenic manner [34]. Remission of disease in animal models of RA [35], multiple sclerosis [36], and type I diabetes [37], has been reported by oral administration of autoantigens. Furthermore, clinical trials have shown signals of efficacy of oral tolerance in autoimmune diseases such as rheumatoid arthritis [38], autoimmune uveitis [39], and multiple sclerosis [40]. In all of these natural conditions of tolerance, common molecules and mechanisms seem to be operating. Accordingly, a natural means of inducing tolerance would be the administration of a “universal donor” cell with tolerogenic potential that generate molecules similar to those found in physiological conditions of tolerance induction.

In some embodiments of the invention the generation of immature dendritic cells by culture with regenerative cells such as amniotic fluid stem cells is performed by either coculture in vitro, or administration in vivo of T regulatory cells [41].

In some embodiments of the invention, alpha 1 antitrypsin is administered in order to induce tolerogenic dendritic cells in order to treat autoimmunity or alloimmunity. The use of this compound for stimulation of immature DC has been previously described and is incorporated by reference [42].

In one embodiment, the invention teaches reduction of Inflammatory cytokines, especially tumor necrosis factor alpha (TNF) and interleukin 1-beta (IL-1), by administration of patient immune cells treated ex vivo with regenerative cells such as amniotic fluid stem cells, wherein in some embodiments said amniotic fluid stem cells are also cultured with immature dendritic cells. It is known that these inflammatory cytokines are major mediators that can elicit changes in cell phenotype, especially causing a variety of morphological and gene expression changes in endothelial cells. In one embodiment treatment of blood vessel abnormalities such as hypercoagulability is treated using patient cells that have been reprogrammed by amniotic fluid stem cells.

In some embodiments of the invention, reprogrammed immune cells are administered together with drugs useful for treatment of inflammatory conditions such as Xigris (activated protein C (APC)) [43]. This protein we claim synergizes with the anti-inflammatory effects of patient reprogrammed immune cells by activating endothelial cell-protecting mechanisms mediating protection against apoptosis, stimulation of barrier function through the angiopoietin/Tie-2 axis, and by reducing local clotting [44-46]. The basis of approval for Xigris has been questioned by some [47] and, additionally, it is often counter-indicated in oncology-associated sepsis (especially leukemias where bleeding is an issue of great concern). In fact, in the Phase III trials of Xigris, hematopoietic transplant patients were excluded [48]. Thus there is a great need for progress in the area of SIRS treatment and adjuvant approaches for agents such as Xigris. In one embodiment of the invention, APC is administered as Xigris.

In one embodiment, cells of the invention are used to treated systemic inflammatory response syndrome (SIRS). One of the main causes of death related to SIRS is dysfunction of the microcirculatory system, which in the most advanced stages is manifested as disseminated intravascular coagulation (DIC) [49]. In one embodiment, patient immune cells that have been reprogrammed with amniotic fluid stem cells are administered together with immature dendritic cells to inhibit onset of DIC. Without being bound to theory, immature dendritic cells are generated in a manner to inhibit inflammatory mediators associated with SIRS, whether endotoxin or injury-related signals such as TLR agonists or HMGB-1, are all capable of activating endothelium systemically [50, 51]. Under physiological conditions, the endothelial response to such mediators is local and provides a useful mechanism for sequestering an infection and allowing immune attack. In SIRS, the fact that the response is systemic causes disastrous consequences including organ failure. The characteristics of this endothelial response include: a) upregulation of tissue factor (TF) [52, 53] and suppression of endothelial inhibitors of coagulation such as protein C and the antithrombin system causing a pro-coagulant state [54]; b) increased expression of adhesion molecules which elicit, in turn, neutrophil extravasation [55]; c) decreased fibrinolytic capacity [56-58]; and d) increased vascular permeability/non-responsiveness to vaso-dilators and vasoconstrictors [59, 60]. Excellent detailed reviews of molecular signals associated with SIRS-induced endothelial dysfunction have been published[61-69] and one of the key factors implicated has been NF-kB [70]. Nuclear translocation of NF-kB is associated with endothelial upregulation of pro-thrombotic molecules and suppressed fibrinolysis [71-73]. In an elegant study, Song et al. inhibited NF-kB selectively in the endothelium by creation of transgenic mice transgenic expressing exogenous i-kappa B (the NF-kB inhibitor) specifically in the vasculature. In contrast to wild-type animals, the endothelial cells of these transgenic mice experienced substantially reduced expression of tissue factor while retaining expression of endothelial protein C receptor and thrombomodulin subsequent to endotoxin challenge. Furthermore, expression of NF-B was associated with generation of TNF-alpha as a result of TACE activity [74]. It is interesting that the beneficial effects of Xigris in SIRS appear to be associated with its ability to prevent the endothelial dysfunction [75] associated with suppression of proinflammatory chemokines [76], prevention of endothelial cell apoptosis [77], and increased endothelial fibrinolytic activity [78, 79]. Some of the protective activities of Xigris have been ascribed to its ability to suppress NF-kB activation in endothelial cells [80, 81].

In one embodiment of the invention, mesenchymal stem cells are utilized to generate Treg, such as in the following examples, and said Treg, pure or unpure, are utilized for reduction of kidney failure. Examples provided before are incorporated by reference.

BM-MSC Induce Treg

In one of the first studies examine whether mesenchymal stem cells (MSC) can induce T regulatory cells, Prevosto et al used MSC obtained from the bone marrow of four healthy donors and nine patients with acute leukemia in complete remission following chemotherapy. Short-term (4 days) co-cultures of MSC and autologous or allogeneic peripheral blood mononuclear cells (PBMC) were set up, the lymphocytes harvested and their regulatory activity assessed. It was found that lymphocytes harvested from MSC-PBMC co-cultures strongly inhibit (up to 95%) mixed lymphocyte reaction (MLR), recall to alloantigen, and CD3- or PHA-induced lymphocyte proliferation. These lymphocytes, termed regulatory cells (Regc), were all CD45+CD2+ with variable proportions of CD25+ cells (range 40-75% n=10) and a minor fraction expressed CTLA4 (2-4%, n=10) or glucocorticoid-induced tumor necrosis factor receptor-related gene (0.5-4% n=10). Both CD4+ and CD8+ Regc purified from MSC-PBMC co-cultures strongly inhibited lymphocyte proliferation at a 1:100 Regc:responder cell ratio. CD4+ Regc expressed high levels of forkhead box P3 (Foxp3) mRNA while CD8+ Regc did not. The effectiveness of Regc, whether CD4+ or CD8+, was 100-fold higher than that of CD4+CD25+ high regulatory T cells. Regc were also generated from highly purified CD25-PBMC or CD4+ or CD8+ T cell subsets. Soluble factors, such as interleukin-10, transforming growth factor-b and prostaglandin E2 did not appear to be involved in the generation of Regc or in the Regc-mediated immunosuppressive effect. Furthermore, cyclosporin A did not affect Regc generation or the immunosuppression induced by Regc [82].

Mechanistically, it appears that HLA-G is required for MSC to stimulate Treg cells. It was shown that MSCs secrete the soluble isoform HLA-G5 and that such secretion is interleukin-10-dependent. Moreover, cell contact between MSCs and allostimulated T cells is required to obtain a full HLA-G5 secretion and, as consequence, a full immunomodulation from MSCs. Blocking experiments using neutralizing anti-HLA-G antibody demonstrate that HLA-G5 contributes first to the suppression of allogeneic T-cell proliferation and then to the expansion of CD4(+)CD25(high)FOXP3(+) regulatory T cells [83].

Another mechanism that seems important is production of leukemia inhibitory factor by the MSC. There was a 7-fold increase (611 pg/ml) in LIF in MSC/MLR as compared to MSCs alone. Using LIF neutralizing antibody, a significant restoration of up to 91% of CD3+ lymphocyte proliferation in MSC/MLR was observed (p=0.021). LIF was implicated in the generation of regulatory lymphocytes, as demonstrated by decrease of Foxp3+ regulatory cells after using LIF neutralizing antibody in MSC/MLR (p=0.06) by flow cytometry. A positive correlation between LIF and human leukocyte antigen (HLA-G) gene expression by MSCs was found (R(2)=0.74) [84].

Allogeneic MSC were shown to induce forkhead box P3 (FoxP3)+ and CD25+ mRNA and protein expression in CD4+ T cells. This phenomenon required direct contact between MSC and purified T cells, although cell contact was not required for MSC induction of FoxP3 expression in an unseparated mononuclear cell population. In addition, through use of antagonists and neutralizing antibodies, MSC-derived prostaglandins and transforming growth factor (TGF)-beta1 were shown to have a non-redundant role in the induction of CD4+CD25+FoxP3+ T cells. Purified CD4+CD25+ T cells induced by MSC co-culture expressed TGF-beta1 and were able to suppress alloantigen-driven proliferative responses in mixed lymphocyte reaction [85].

One study showed that MSC actually convert Th17 cells into Treg-like cells. Investigators showed that MSCs induced the production of IL-10 and trimethylation of histone H3K4me3 at the promoter of the FOXP3 gene locus, whereas it suppressed trimethylation of the corresponding region in the RORC gene in Th17 cells. These epigenetic changes were associated with the induction of fork head box p3 and the acquisition by Th17 cells of the capacity to inhibit in vitro proliferative responses of activated CD4(+) T cells, which was enhanced when MSCs were preincubated with IFN-gamma and TNF-alpha [86].

Another study showed that MSCs and their products effectively regulate expression of transcription factors Foxp3 and RORγt and control the development of Tregs and Th17 cells in a population of alloantigen-activated mouse spleen cells or purified CD4(+)CD25(−) T-cells. The immunomodulatory effects of MSCs were more pronounced when these cells were stimulated to secrete TGF-β alone or TGF-β together with IL-6. Unstimulated MSCs produce TGF-β, but not IL-6, and the production of TGF-β can be further enhanced by the anti-inflammatory cytokines IL-10 or TGF-β. In the presence of proinflammatory cytokines, MSCs secrete significant levels of IL-6, in addition to a spontaneous production of TGF-β. MSCs producing TGF-β induced preferentially expression of Foxp3 and activation of Tregs, whereas MSC supernatants containing TGF-β together with IL-6 supported RORγt expression and development of Th17 cells. The effects of MSC supernatants were blocked by the inclusion of neutralization monoclonal antibody anti-TGF-β or anti-IL-6 into the culture system. The results showed that MSCs represent important players that reciprocally regulate the development and differentiation of uncommitted naive T-cells into anti-inflammatory Foxp3(+) Tregs or proinflammatory RORγt(+) Th17 cell population and thereby can modulate autoimmune, immunopathological, and transplantation reactions [87].

In another study, using an in vitro culture system, researchers showed that culture-expanded bone marrow-derived MSC promote the generation of CD4(+) CD25(hi) FoxP3(+) T cells in human PBMC populations and that these populations are functionally suppressive. Similar results were obtained with MSC-conditioned medium, indicating that this process is dependent on soluble factors secreted by the MSC. Antibody neutralization studies showed that TGF-β1 mediates induction of Tregs. TGF-β1 is constitutively secreted by MSC, suggesting that the MSC-induced generation of Tregs by TGF-β1 was independent of the interaction between MSC and PBMC. Monocyte-depletion studies showed that monocytes are indispensable for MSC-induced Treg formation. MSC promote the survival of monocytes and induce differentiation toward macrophage type 2 cells that express CD206 and CD163 and secrete high levels of IL-10 and CCL-18, which is mediated by as yet unidentified MSC-derived soluble factors. CCL18 proved to be responsible for the observed Treg induction. These data indicate that MSC promote the generation of Tregs. Both the direct pathway through the constitutive production of TGF-β1 and the indirect novel pathway involving the differentiation of monocytes toward CCL18 producing type 2 macrophages are essential for the generation of Tregs induced by MSC [88].

Another study MSCs were obtained from mouse bone marrow and characterized according to their surface antigen expression and their multilineage differentiation potential. CD4(+) T cells isolated from mouse spleens were induced to differentiate into Th1 or Th17 cells and co-cultured with MSCs added at day 0, 2 or 4 of the differentiation processes. After six days, CD25, Foxp3, IL-17 and IFN-γ expression was assessed by flow cytometry and helios and neuropilin 1 mRNA levels were assessed by RT-qPCR. For the functional assays, the ‘conditioned’ subpopulation generated in the presence of MSCs was cultured with concanavalin A-activated CD4(+) T cells labeled with carboxyfluorescein succinimidyl ester. Finally, we used the encephalomyelitis autoimmune diseases (EAE) mouse model, in which mice were injected with MSCs at day 18 and 30 after immunization. At day 50, the mice were euthanized and draining lymph nodes were extracted for Th1, Th17 and Treg detection by flow cytometry. It was shown that MSCs were able to suppress the proliferation, activation and differentiation of CD4(+) T cells induced to differentiate into Th1 and Th17 cells. This substantial suppressive effect was associated with an increase of the percentage of functional induced CD4(+)CD25(+)Foxp3(+) regulatory T cells and IL-10 secretion. However, using mature Th1 or Th17 cells our results demonstrated that while MSCs suppress the proliferation and phenotype of mature Th1 and Th17 cells they did not generate Treg cells. Finally, we showed that the beneficial effect observed following MSC injection in an EAE mouse model was associated with the suppression of Th17 cells and an increase in the percentage of CD4(+)CD25(+)Foxp3(+) T lymphocytes when administrated at early stages of the disease. This study demonstrated that MSCs contribute to the generation of an immunosuppressive environment via the inhibition of proinflammatory T cells and the induction of T cells with a regulatory phenotype [89].

investigated the immunomodulatory effect of MSCs on peripheral blood mononuclear cells (PBMCs) in ALS patients, focusing on regulatory T lymphocytes (Treg; CD4(+) /CD25(high)/FoxP3(+)) and the mRNA expression of several cytokines (IFN-γ, TNF-α, IL-17, IL-4, IL-10, IL-13, and TGF-β). Peripheral blood samples were obtained from nine healthy controls (HC) and sixteen patients who were diagnosed with definite or probable ALS. Isolated PBMCs from the blood samples of all subjects were co-cultured with MSCs for 24 or 72 h. Based on a fluorescence-activated cell sorting analysis, we found that co-culture with MSCs increased the Treg/total T-lymphocyte ratio in the PBMCs from both groups according to the co-culture duration. Co-culture of PBMCs with MSCs for 24 h led to elevated mRNA levels of IFN-γ and IL-10 in the PBMCs from both groups. However, after co-culturing for 72 h, although the IFN-γ mRNA level had returned to the basal level in co-cultured HC PBMCs, the IFN-γ mRNA level in co-cultured ALS PBMCs remained elevated. Additionally, the levels of IL-4 and TGF-β were markedly elevated, along with Gata3 mRNA, a Th2 transcription factor mRNA, in both HC and ALS PBMCs co-cultured for 72 h. The elevated expression of these cytokines in the co-culture supernatant was confirmed via ELISA. Furthermore, we found that the increased mRNA level of indoleamine 2,3-dioxygenase (IDO) in the co-cultured MSCs was correlated with the increase in Treg induction. These findings of Treg induction and increased anti-inflammatory cytokine expression in co-cultured ALS PBMCs provide indirect evidence that MSCs may play a role in the immunomodulation of inflammatory responses when MSC therapy is targeted to ALS patients. We propose the following mechanism for the effect of mesenchymal stem cells (MSCs) administered intrathecally in amyotrophic lateral sclerosis (ALS): MSCs increase infiltration of peripheral immune cells into CNS and skew the infiltrated immune cells toward regulatory T lymphocytes (Treg) and Th2 lymphocytes. Treg and Th2 secret anti-inflammatory cytokines such as IL-4, IL-10, and TGF-β. A series of immunomodulatory mechanism provides a new strategy for ALS treatment [90].

Allogeneic bone marrow mesenchymal stromal cells were cultured with T cells or dendritic cells in the presence or absence of gamma secretase inhibitor to block Notch receptor signalling. T cells and dendritic cells were examined by flow cytometry for changes in phenotype marker expression. Stable knock down MSC were generated to examine the influence of Jagged 1 signalling by MSC. Both wildtype and knockdown MSC were subsequently used in vivo in an animal model of allergic airway inflammation. The Notch ligand Jagged-1 was demonstrated to be involved in MSC expansion of regulatory T cells (Treg). Additionally, MSC-induced a functional semi-mature DC phenotype, which further required Notch signalling for the expansion of Treg. MSC, but not Jagged-1 knock down MSC, reduced pathology in a mouse model of allergic airway inflammation. Protection mediated by MSC was associated with enhanced Treg in the lung and significantly increased production of interleukin (IL)-10 in splenocytes re-stimulated with allergen. Significantly less Treg and IL-10 was observed in mice treated with Jagged-1 knock down MSC. The current study suggests that MSC-mediated immune modulation involves the education and expansion of regulatory immune cells in a Jagged-1 dependent manner and provides the first report of the importance of Jagged-1 signalling in MSC protection against inflammation in vivo [91].

In another study modified human bone marrow-derived MSC with interleukin-17A (MSC-17) to enhance T cell immunosuppression but not their immunogenicity. MSC-17, unlike MSC-γ, showed no induction or upregulation of MHC class I, MHC class II, and T cell costimulatory molecule CD40, but maintained normal MSC morphology and phenotypic marker expression. When cocultured with phytohemagglutinin (PHA)-activated human T cells, MSCs-17 were potent suppressors of T cell proliferation. Furthermore, MSC-17 inhibited surface CD25 expression and suppressed the elaboration of Th1 cytokines, IFN-γ, tumor necrosis factor-α (TNF-α), and IL-2 when compared with untreated MSCs (UT-MSCs). T cell suppression by MSC-17 correlated with increased IL-6 but not with indoleamine 2,3-dioxygenase 1, cyclooxygenase 1, and transforming growth factor β-1. MSC-17 but not MSC-γ consistently induced CD4(+) CD25(high) CD127(low) FoxP3(+) regulatory T cells (iTregs) from PHA-activated CD4(+) CD25(−) T cells. MSC-induced iTregs expressed CD39, CD73, CD69, OX40, cytotoxic T-lymphocyte associated antigen-4 (CTLA-4), and glucocorticoid-induced TNFR-related protein (GITR). These suppressive MSCs-17 can engender Tregs to potently suppress T cell activation with minimal immunogenicity and thus represent a superior T cell immunomodulator for clinical application [92].

Mechanistically, it was shown that microRNAs may be involved in MSC mediated generation of Treg cells. In one study, the possible effect of MSCs on miR-126a and miR-10a expression in iTregs and, consequently on Foxp3 stability, were investigated. It was first demonstrated that in vitro MSC-iTreg generation was directly associated with strong modifications of miR-126a. Subsequently, infused high doses of MSCs in a murine model of allogeneic skin transplantation (C57BL/6 into Balb/c). This treatment significantly prolonged skin allograft survival compared to PBS treated mice. When splenocytes from grafted mice were collected, we observed that the expression of Foxp3 gene was elevated at day 5 and 10 post-graft merely in MSCs treated mice. Moreover, Foxp3 expression was not associated with modified miR-10a expression comparable to in vitro experiments. Thus, our data identify a solid mechanism where MSCs induce conversion of conventional T cells to iTregs through strong modifications of miR-126a. Although miR-10a expression level remains unchanged in vitro and in vivo, we observed expression of this miR in MSC-DC condition [93].

In another mechanistic study, Human naive CD4+ T cells were stimulated with anti-CD3/28 Abs and cocultured with human MSC culture supernatant for 48 h. The proliferation and cytokine production of CD4+ T cells and surface molecule expression on CD4+ T cells were evaluated. The proliferation of anti-CD3/28 Abs-stimulated CD4+ T cells was suppressed by the addition of human MSC culture supernatant; in addition, the production of IL-10 and IL-4 increased. The human MSC culture supernatant induced CD4+FOXP3+ Tregs that expressed CD25, CTLA-4, glucocorticoid-induced TNFR-related protein, insulin-like growth factor (IGF)-1R, and IGF-2R, showing antiproliferative activity against CD4+ T cells. In addition, the induction of Tregs by human MSC culture supernatant was enhanced by the addition of IGF and suppressed by the inhibition of IGF-1R. In contrast, a significant amount of IGF binding protein (IGFBP)-4, an inhibitor of IGF action, was detected in the human MSC culture supernatant. After neutralization of IGFBP-4 in the human MSC culture supernatant by anti-IGFBP-4 Ab, Treg numbers increased significantly. Thus, our results raise the possibility that human MSC actions also involve a negative-regulatory mechanism that suppresses Treg proliferation by releasing IGFBP-4. The results of this study suggest that regulation of IGF may be important for treatments using human MSCs [94].

Another mechanistic study evaluated the possible effect of MSCs on the mRNA expression of Runx complex genes (Runx1, Runx3, and CBFB) that perch on TSDR in iTregs and play the main role in suppressive properties of Tregs, a regulatory pathway that has not yet been explored by MSCs. Also, we investigated the mRNA expression of MBD2 that promotes TSDR demethylation in Tregs. The authors showed that in vitro MSC-iTreg induction was associated with strong mRNA modifications of genes involved in Runx complex [95].

In some studies it appears that MSC exosomes are involved in Treg generation. In one experiment, MSC exosomes were incubated with mouse spleen CD4+ T cells that were activated with either anti-CD3/CD28 mAbs or allogenic antigen-presenting cell (APC)-enriched spleen CD11c+ cells to determine whether production of mouse CD4+CD25+ T cells or CD4+CD25+Foxp3+ Tregs could be induced. MSC exosomes were also administered to the lethal chimeric human-SCID mouse model of graft-versus-host disease (GVHD) in which human peripheral blood mononuclear cells were infused into irradiated NSG mice to induce GVHD. It was demonstrated MSC exosome-induced production of CD4+CD25+ T cells or CD4+CD25+Foxp3+ Tregs from CD4+ T cells activated by allogeneic APC-enriched CD11C+ cells but not those activated by anti-CD3/CD28 mAbs. This induction was exosome- and APC dose-dependent. In the mouse GVHD model in which GVHD was induced by transplanted human APC-stimulated human anti-mouse CD4+ T cell effectors, MSC exosome alleviated GVHD symptoms and increased survival. Surviving exosome-treated mice had a significantly higher level of human CD4+CD25+CD127low/− Tregs than surviving mice treated with Etanercept, a tumor necrosis factor inhibitor [96].

Mechanisms of FoxP3 upregulation in Treg cells generated by MSC were studied in a report describing the role of cell-cell contact and cytokine secretion by bone marrow-derived MSCs (BM-MSCs) on the induction, stability, and suppressive functions of Tregs under various experimental conditions that lead to Foxp3 generation by flow cytometry and ELISA respectively. Second, the report studied the effect of MSCs on TRAF6, GRAIL, USP7, STUB1, and UBC13 mRNA expression in CD4+ T cells in correlation with the suppressive function of iTregs by real-time PCR; also, the authors investigated Foxp3 Treg-specific demethylated region (TSDR) methylation in correlation with Foxp3 stability by the high-resolution melting technique. Third, the authors examined the effect of ex-vivo-expanded BM-MSCs on the induction of transplant tolerance in a model of fully allogeneic skin transplantation. We further analyzed the cytokine secretion patterns in grafted mice as well as the mRNA expression of ubiquitination genes in CD4+ T cells collected from the spleens of protected mice. It was found that in-vitro MSC-induced Tregs express high mRNA levels of ubiquitination genes such as TRAF6, GRAIL, and USP7 and low levels of STUB1. Moreover, they have enhanced TSDR demethylation. Infusion of MSCs in a murine model of allogeneic skin transplantation prolonged allograft survival. When CD4+ T cells were harvested from the spleens of grafted mice, we observed that mRNA expression of the Foxp3 gene was elevated. Furthermore, Foxp3 mRNA expression was associated with increased TRAF6, GRAIL, UBC13, and USP7 and decreased STUB1 mRNA levels compared with the levels observed in vitro [97].

In another mechanistic study, assessment of whether MSC-mediated transfer of mitochondria (MitoT) to immune cells to generate Treg cell was assessed. The authors describe dose-dependent MitoT from mitochondria-labeled MSCs mainly to CD4+ T cells, rather than CD8+ T cells or CD19+ B cells. Artificial transfer of isolated MSC-derived mitochondria increases the expression of mRNA transcripts involved in T-cell activation and T regulatory cell differentiation including FOXP3, IL2RA, CTLA4, and TGFβ1, leading to an increase in a highly suppressive CD25+FoxP3+ population. In a GVHD mouse model, transplantation of MitoT-induced human T cells leads to significant improvement in survival and reduction in tissue damage and organ T CD4+, CD8+, and IFN-γ+ expressing cell infiltration. These findings point to a unique CD4+ T-cell reprogramming mechanism with pre-clinical proof-of-concept data that pave the way for the exploration of organelle-based therapies in immune diseases.

In an experiment focused on hepatocyte growth factor as a potential mechanism of MSC being able to stimulate Treg, scientists investigated the effects of MSCs on the differentiation of CD4+ T cells and the functions of Th17/Treg cells in response to LPS stimulation by performing in vitro coculture experiments. MSCs were added to the upper chambers of cell culture inserts, and CD4+ T cells were plated in the lower chambers, followed by treatment with LPS or an anti-HGF antibody. Th17 (CD4+CD3+RORrt+) and Treg (CD4+CD25+Foxp3+) cell frequencies were analysed by flow cytometry, and the expression of Th17 cell- and Treg cell-related cytokines in the CD4+ T cells or culture medium was measured by quantitative PCR (qPCR) and enzyme-linked immunosorbent assay (ELISA), respectively. Neutrophil functions were determined by flow cytometry after a coculture with Th17/Treg cells. The percentage of CD4+CD25+Foxp3+ cells was significantly increased in the CD4+ T cell population, while the percentage of CD4+CD3+RORrt+ cells was significantly decreased after MSC coculture. However, the MSC-induced effect was significantly inhibited by the anti-HGF antibody (p<0.05). Furthermore, MSCs significantly inhibited the CD4+ T cell expression of IL-17 and IL-6 but increased the expression of IL-10 (p<0.05 or p<0.01); these effects were inhibited by the anti-HGF antibody (p<0.05). In addition, CD4+ T cells cocultured with MSCs significantly inhibited neutrophil phagocytic and oxidative burst activities (p<0.05 or p<0.01); however, these MSC-induced effects were inhibited by the anti-HGF antibody (p<0.05). The authors concluded that MSCs induced the conversion of fully differentiated Th17 cells into functional Treg cells and thereby modulated the Th17/Treg cell balance in the CD4+ T cell population, which was partly attributed to HGF secreted by the MSCs [98].

Another study showed that unlike WT-T cells, their TNFR2 KO counterparts are remarkably less able to convert into Foxp3+ and Foxp3− Tregs. Furthermore, TNFR2 blockade diminished the anti-inflammatory cytokine secretion by iTregs and consequently resulted in less T cell immunosuppression. This work is the first evidence of the crucial association of TNFR2 expression by T cells with their iTreg conversion capacity by MSCs. It strengthens once more the potential of anti-TNFR2 administration for a strong and effective interference with the immunosuppression exerted by TNFR2-expressing cells [99].

Adipose MSC Induce Treg

The suppressive activity of hASCs was cell-to-cell contact dependent and independent. hASCs also stimulated the generation of FoxP3 protein-expressing CD4(+)CD25(+) regulatory T cells, with the capacity to suppress collagen-specific T cell responses [100].

Stimulation of FoxP3 in T cells by adipose MSC was also demonstrated in another study in which subpopulations of allo-activated T cells where shown to be capable of binding to human adipose-derived stromal cells (ASC). The bound T-cell population contained CD8+ T cells and was enriched for CD4-CD8− T cells, whereas the proportion of CD4+ T cells was decreased compared with the non-bound T-cell population. Bound CD4+ T cells had high proliferative activity and increased CD25 and FoxP3 expression. However, they also expressed CD127, excluding regulatory T-cell function [101].

Engela et al. used perirenal adipose-tissue derived MSC (ASC) obtained from kidney donors induced a 2.1-fold increase in the percentage of CD25(+) CD127(−) FoxP3(+) cells within the CD4(+) T cell population from allostimulated CD25(−/dim) cells. Interleukin (IL)-2 receptor blocking prevented this induction. The ASC-induced T cells (iT(reg)) inhibited effector cell proliferation as effectively as nT(reg). The vast majority of cells within the iT(reg) fraction had a methylated FOXP3 gene T(reg)-specific demethylated region (TSDR) indicating that they were not of nT(reg) origin. In conclusion, ASC induce T(reg) from effector T cells. These iT(reg) have immunosuppressive capacities comparable to those of nT(reg). Their induction is IL-2 pathway-dependent [102].

Frazier et al. show that ASCs stimulated proliferation of naive CD4(+) T cells and the percentage of CD25(+) T cells was significantly increased under both low and ambient 02. Forkhead box P3 (FoxP3) and transforming growth factor beta (TGF-β) mRNA expression were significantly increased when ASCs were cocultured with CD4(+) T cells under low compared with ambient 02 conditions. The low 02-induced regulatory T cells (iTregs) exhibited functionality when added to mixed lymphocyte reactions as demonstrated by inhibition of peripheral blood mononuclear cell proliferation, and by >300-fold increase in FoxP3 mRNA, and >2-fold increase in TGF-β mRNA expression. These results demonstrate that under physiologically relevant low 02 conditions, direct contact of human ASCs with naive CD4(+) T cells induced functional iTregs [103].

To determine the effect of the immunomodulatory and regulatory functions of adipose-derived MSCs (AD-MSCs) on C57BL/6 spleen-isolated mononuclear cells (Spleen-MNCs), the gene expression of well-known effector and regulatory Th cell-related transcription factors, i.e., t-bet, GATA-3, Ror-γt and Foxp3, and their related cytokines, i.e., IFN-γ for Th1 cells, IL-4 for Th2 cells, IL-17 for Th17 cells and IL-10 and TGF-β for regulatory T cells, was studied using a co-culture condition system. The proliferation index of Spleen-MNCs was analyzed using a cell proliferation assay kit that utilized the CFSE staining method. Our findings indicate that AD-MSCs greatly impact the up-regulation of immunomodulatory cytokines, such as TGF-β (p<0.001), and the down-regulation of inflammatory cytokines, such as IFN-γ (p<0.005), and transcription factors, such as t-bet (p<0.001). Considering the immunomodulatory effects of MSCs in the differentiation of Th cell subsets, understanding and harnessing this property of MSCs could be a powerful strategy in the treatment of inflammatory autoimmune diseases such as multiple sclerosis [104].

One study examine 17 patients with active disease matching the ACR/EULAR 2010 criteria for. Patients' PBMC were cultured in AT-MSC-conditioned medium (AT-MSCcm) and in control medium. The cytokine production of AT-MSC and PBMC was quantified by ELISA. Th17 and Treg were determined by flow cytometry. AT-MSCcm contained: IL-6, IL-17, IL-21, CCL2, CCL5, IL-8, sVEGF-A and PGE2. Cultivation of patients' PBMC with AT-MSCcm increased TGF-β1 (8318 pg/ml; IQR 6327-11,686) vs control medium [6227 pg/ml (IQR 1681-10,148, p=0.013)]. PBMC cultivated with AT-MSCcm downregulated TNF-α, IL-17A, and IL-21 compared to control PBMC: 5 pg/ml IQR (1.75-11.65) vs 1 pg/ml (IQR 0.7-1.9), p=0.001; 4.2 pg/ml (IQR 3.1-6.1) vs 2.3 pg/ml (IQR.75-5.42), p=0.017; 66.9 pg/ml (IQR 40.6-107.2) vs 53 pg/ml (IQR 22-73), p=0.022. Th17 decreased under the influence of AT-MSCcm: 10.13±3.88% vs 8.98±3.58%, p=0.02. CD4+FoxP3+, CD4+CD25-FoxP3+, and CD4+CD25+FoxP3+ was 11.35±4.1%; 7.13±3.12% and 4.22±2% in control PBMC. Accordingly, CD4+FoxP3+, CD4+CD25-FoxP3+, and CD4+CD25+FoxP3+ significantly increased in PBMC cultured with AT-MSCcm: 15.6±6.1%, p=0.001; 9.56±5.4%, p=0.004 and 6.04±3.6%, p=0.001 [105].

In another study breast adipose tissues of a breast cancer patient and a normal individual were used. Magnetic cell sorting (MACS) was employed for purifying naïve CD4+ T cells from peripheral blood of five healthy donors. Naïve CD4+ T cells were then co-cultured with ASCs for five days. The phenotype of regulatory T cells (Tregs) and production of interleukine-10 (IL-10), transforming growth factor beta (TGF-β) and IL-17 were assessed using flow cytometry and ELISPOT assays, respectively. CD4+CD25-FOXP3+CD45RA+ Tregs were expanded in the presence of cancer ASCs but CD4+CD25+Foxp3+CD45RA+ regulatory T cells were up-regulated in the presence of both cancer- and normal-ASCs. This up-regulation was statistically significant in breast cancer-ASCs compared to the cells cultured without ASCs (P=0.002). CD4+CD25+FOXP3+Helios+, CD4+CD25− FOXP3+Helios+ and CD25+FOXP3+CD73+CD39+ Tregs were expanded after co-culturing of T cells with both cancer-ASCs and normal-ASCs, while they were statistically significant only in the presence of cancer-ASCs (P<0.05). Production of IL-10, IL-17 and TGF-β by T cells was increased in the presence of either normal- or cancer-ASCs; however, significant effect was only observed in the IL-10 and TGF-β of cancer-ASCs (P<0.05). The results further confirm the immunosuppressive impacts of ASCs on T lymphocytes and direct them to specific regulatory phenotypes which may support immune evasion and tumor growth [106].

A comparative evaluation of the effects of adipose tissue derived MSC (ASCs) on the mitogen-stimulated T cells at the ambient (20%) and tissue-related (5%) 02 levels demonstrated reduced T cell activation by the HLA-DR expression, decreased pro-inflammatory and increased anti-inflammatory cytokine production in co-culture, inhibited T cell proliferation, with the effects increased at hypoxia. T cell interactions with ASCs resulted in the up-regulation of PDCD1, Foxp3, and TGFβ1 known to play an important role in the immune response suppression, and in the down-regulation of genes involved in the inflammatory reaction (IL2, IFNG). These changes were significantly increased under hypoxia [107].

Importance of exosomes in MSC generation of Treg was shown in a study that evaluated whether mesenchymal stromal cell extracellular vesicles (MSC-EVs) can modulate T cell response. MSCs were expanded and EVs were obtained by differential ultracentrifugation of the supernatant. The incorporation of MSC-EVs by T cells was detected by confocal microscopy. Expression of surface markers was detected by flow cytometry or CytoFLEX and cytokines were detected by RT-PCR, FACS and confocal microscopy and a miRNA PCR array was performed. We demonstrated that MSC-EVs were incorporated by lymphocytes in vitro and decreased T cell proliferation and Th1 differentiation. Interestingly, in Th1 polarization, MSC-EVs increased Foxp3 expression and generated a subpopulation of IFN-γ+/Foxp3+ T cells with suppressive capacity. A differential expression profile of miRNAs in MSC-EVs-treated Th1 cells was seen, and also a modulation of one of their target genes, TGFbR2. MSC-EVs altered the metabolism of Th1-differentiated T cells, suggesting the involvement of the TGF-β pathway in this metabolic modulation. The addition of MSC-EVs in vivo, in an OVA immunization model, generated cells Foxp3+ [108].

In another study human peripheral blood mononuclear cells from healthy donors were cocultured with allogeneic bone marrow-derived MSCs expanded under xenogeneic-free conditions. Our data show an increase in the counts and frequency of CD4+CD25high Foxp3+CD127low Treg cells (4- and 6-fold, respectively) after a 14-day coculture. However, natural Treg do not proliferate in coculture with MSCs. When purified conventional CD4 T cells (Tcon) are cocultured with MSCs, only cells that acquire a Treg-like phenotype proliferate. These MSC-induced Treg-like cells also resemble Treg functionally, since they suppress autologous Tcon proliferation. Importantly, the DNA methylation profile of MSC-induced Treg-like cells more closely resembles that of natural Treg than of Tcon, indicating that this population is stable. The expression of PD-1 is higher in Treg-like cells than in Tcon, whereas the frequency of PDL-1 increases in MSCs after coculture. TGF-β levels are also significantly increased MSC cocultures. Overall, our data suggest that Treg enrichment by MSCs results from Tcon conversion into Treg-like cells, rather than to expansion of natural Treg, possibly through mechanisms involving TGF-β and/or PD-1/PDL-1 expression [109].

To demonstrate adipose MSC are capable of generatin Treg through exosomes . . . exosomes isolated form adipose tissue derived mesenchymal stem cells (AD-MSC-Exo) were transfected with miR-10a and added to naïve T cells purified from mouse spleen. AD-MSC-Exos were characterized and the efficacy of miR-10a delivery was evaluated. The expression levels of T-bet, GATA3, RORγt, and Foxp3 and the secreted levels of IFN-γ, IL-4, IL-17, and TGF-β respectively specific to Th1, Th2, Th17 and Treg, were assessed by qPCR and ELISA. Being transferred by AD-MSC-Exo, miR-10a was effectively induced in CD4+ T cells. Upon treatment with miR-10a loaded exosomes, the expression levels of RORγt and Foxp3 were enhanced and that of T-bet was reduced. Similarly, the secreted levels of IL-17, and TGF-β were increased and that of IFN-γ was decreased. These data indicate that miR-10a loaded exosomes, promote Th17 and Tregs response while reduce that of Th1. Promotion of both Th17 and Tregs in concert, mediated by the combined effect of miR-10a and MSC-Exo, indicate new therapeutic potentials, particularly in line with novel anti-tumor immunotherapeutic strategies [110].

In another study, isolated human peripheral blood CD4+ T cells and co-cultured them with Ad-MSCs at a ratio of 4:1 under either Th17 or Treg cell polarizing conditions for 4 days to detect the proportions of IL-17-producing CD4+ T (Th17) and CD4+CD25+Foxp3+ regulatory T (Treg) cells by flow cytometry. We also determined the mRNA expression levels of the retinoid-related orphan nuclear receptor (RORγt) transcription factor and those of interleukin-6 receptor (IL-6R), interleukin-23 receptor (IL-23R), leukemia inhibitory factor (LIF), and LIF receptor (LIFR) by quantitative reverse transcription PCR. We detected levels of cytokines in the supernatant (including LIF, IL-6, IL-23, IL-10, and TGF-β) using ELISA. These results showed that Ad-MSCs specifically inhibited the differentiation of PBMCs of patients with PD into IL-17-producing CD4+ T cells by decreasing expressions of IL-6R, IL-23R, and RORγt (the key transcription factor for Th17 cells). Moreover, Ad-MSCs induced a functional CD4+CD25+Foxp3+ T regulatory cell phenotype as evidenced by the secretion of IL-10. The levels of IL-6, IL-23, and TGF-β remained constant after co-culture under either the Th17 or the Treg cell polarizing condition. In addition, levels of LIF protein and its receptor mRNA were significantly increased under both polarizing conditions. The present in vitro study found that Ad-MSCs from healthy participants were able to correct the imbalance between Th17 and Treg found in PBMCs of PD patients, which were correlated with an increase in LIF secretion and a decrease in expression of IL-6R, IL-23R, and RORγt [111].

Dental Pulp MSC

Another study examined whether human dental pulp-derived stem cells (hDP-SC) have regulatory effects on phytohemagglutinin (PHA)-activated CD3(+) T cells. The investigators aimed to define the regulatory mechanisms associated with hDP-SC that occur in mixed lymphocyte reaction (MLR) and transwell systems with PHA-CD3(+) T cells and hDP-SC at a ratio of 1:1. Proliferation, apoptosis and pro- and anti-inflammatory cytokines of PHA-CD3(+)T cells, the expression of Regulatory T cells (Treg) markers and some regulatory factors related to hDP-SC, were studied in Both transwell and MLR are co-cultures systems. Anti-proliferative and apoptotic effects of hDP-SC were determined in co-culture systems. Elevated expression levels of human leukocyte antigen (HLA)-G, hepatocyte growth factor (HGF)-β1, intracellular adhesion molecule (ICAM-1)-1, interleukin (IL)-6, IL-10, transforming growth factor (TGF)-β1, vascular adhesion molecule (VCAM)-1 and vascular endothelial growth factor (VEGF) by hDP-SC were detected in the co-culture systems. We observed decreased expression levels of pro-inflammatory cytokines [interferon (IFN)-γ, IL-2, IL-6 receptor (R), IL-12, Interleukin-17A (IL-17A), tumor necrosis factor (TNF)-α] and increased expression levels of anti-inflammatory cytokine [inducible protein (IP)-10] from PHA-CD3(+) T cells in the transwell system. Expression of Treg (CD4(+) CD25(+) Foxp3(+)) markers was significantly induced by hDP-SC in both co-culture systems [112].

To compare the effects of various mesenchymal stem cells, those isolated from human exfoliated deciduous teeth (SHEDs), dental pulp stem cells (DPSCs), and dental follicle stem cells (DFSCs), on human peripheral blood mononuclear cells (PBMCs). Method. Mesenchymal stem cells were isolated from three sources in the orofacial region. Characterization and PCR analyses were performed. Lymphocytes were isolated from healthy peripheral venous blood. Lymphocytes were cocultured with stem cells in the presence and absence of IFN-γ and stimulated with anti-CD2, anti-CD3, and anti-CD28 for 3 days. Then, lymphocyte proliferation, the number of CD4(+)FoxP3(+) T regulatory cells, and the levels of Fas/Fas ligand, IL-4, IL-10, and IFN-γ in the culture supernatant were measured. Results. The DFSCs exhibited an enhanced differentiation capacity and an increased number of CD4(+)FoxP3(+) T lymphocytes and suppressed the proliferation and apoptosis of PBMCs compared with SHEDs and DPSCs. The addition of IFN-γ augmented the proliferation of DFSCs. Furthermore, the DFSCs suppressed IL-4 and IFN-γ cytokine levels and enhanced IL-10 levels compared with the other cell sources. Conclusion. These results suggest that IFN-γ stimulates DFSCs by inducing an immunomodulatory effect on the PBMCs of healthy donors while suppressing apoptosis and proliferation and increasing the number of CD4(+)FoxP3(+) cells [113].

The study was to evaluate the mechanisms of immune tolerance of dental pulp-derived MSC (DP-MSC) in vitro and in vivo. The authors isolated DP-MSCs from human dental pulp and co-cultured them with CD4⁺ T-cells. To evaluate the role of cytokines, we blocked TGF-β and IL-10, separately and together, in co-cultured DP-MSCs and CD4⁺ T-cells. We analyzed CD25 and FoxP3 to identify regulatory T-cells (Tregs) by fluorescence-activated cell sorting (FACS) and real-time PCR. We performed alloskin grafts with and without DP-MSC injection in mice. We performed mixed lymphocyte reactions (MLRs) to check immune tolerance. It was found that co-culture of CD4⁺ T-cells with DP-MSCs increased the number of CD4⁺CD25⁺FoxP3⁺ Tregs (p<0.01). TGF-β or/and IL-10 blocking suppressed Treg induction in co-cultured cells (p<0.05). TGF-β1 mRNA levels were higher in co-cultured DP-MSCs and in co-cultured CD4⁺ T-cells than in the respective monocultured cells. However, IL-10 mRNA levels were not different. There was no difference in alloskin graft survival rate and area between the DP-MSC injection group and the non-injection group. Nonetheless, MLR was reduced in the DP-MSC injected group (p<0.05). The authors concluded that DP-MSCs can modulate immune tolerance by increasing CD4⁺CD25⁺FoxP3⁺ Tregs. TGF-β1 and IL-10 are factors in the immune-tolerance mechanism [114].

Skin MSC Induce Treg

Here we show that plastic-adherent, human dermal cells induce FoxP3 expression in TCR-complex-stimulated CD25(−)CD4(+)CD45RA(+) T cells in the absence of CD28 co-ligation in a cell-contact-dependent manner. These FoxP3(+) T cells reveal an effective suppressive capacity in vitro. Moreover, we found that the vast majority of CD90(+) dermal cells are perivascularly located and generate a significantly higher percentage of regulatory T cells compared with cells expressing markers such as CD271 in vitro. Importantly, we further demonstrate that plastic-adherent dermal cells are also able to differentiate toward the endothelial lineage. Our data show that human skin harbors specific cell types with immunosuppressive potential, which are located in close vicinity to their likely operational area and provide evidence for a CD28-independent regulatory mechanism. Further, the differentiation potential into endothelial cells suggests the existence of a tissue-resident cell pool for vessel regeneration [115].

Decidual Mesenchymal Stem Cells (DSC)

One study investigated factors of importance in the reduction of alloreactivity by DSCs. We found that DSCs need to have cell-cell contact in order to mediate suppression in mixed lymphocyte reactions (MLRs). This contact dependency is consistent with an increased frequency of CD4(+)CD25(high)FOXP3(+) regulatory T cells (Tregs) and an augmented intensity of CD25 expression in CD4(+) T cells. Blocking of the activity of indoleamine-2,3-dioxygenase (IDO), prostaglandin E2, PD-L1, and IFN-γ impaired the antiproliferative ability of the DSCs in MLRs. Neutralization of IDO also reduced the frequency of Tregs. In contrast to BM-MSCs, pretreatment of DSCs with high concentrations of IFN-γ (100 U/mL) reduced their ability to suppress alloreactivity, but stimulation of DSCs with MLR supernatants containing low levels of IFN-γ had no effect on the suppressive capacity in MLR. To conclude, DSCs differ in several aspects from MSCs and need to be close to alloreactive lymphocytes to mediate a suppressive effect and increase the frequency of Tregs [116].

Placental MSC induce Treg

effects of interferon (IFN)-γ on human placenta-derived mesenchymal stromal cells (hPMSCs), in particular, their adhesion, proliferation and migration and modulatory effects on the CD4+CXCR5+Foxp3+ Treg subset. And we compared hPMSCs ability to induce the generation of different Treg subsets in response to treatment with IFN-γ. We found that IFN-γ suppressed the proliferation and migration for hPMSCs. The ability of hPMSCs to induce the generation of CD4+CXCR5+Foxp3+ Treg subset was enhanced by IFN-γ. And maximal effectiveness of IFN-γ treated hPMSCs upon inducing the generation of Treg subsets was for CD4+CXCR5+Foxp3+ Treg subset as compared with that of CD4+CD25+Foxp3+, CD8+CD25+Foxp3+, CD4+IL-10+ and CD8+IL-10+ Treg subsets [117].

In another study, authors compared the expression of FoxP3 in activated T cells isolated from peripheral blood and co-cultured with PD-MSCs or bone marrow-derived mesenchymal stem cells (BM-MSCs) and analyzed their effect on T cell proliferation and cytokine profiles. Additionally, we verified the immunomodulatory function of PD-MSCs by siRNA-mediated silencing of FoxP3. MSCs, including PD-MSCs and BM-MSCs, promoted differentiation of naive peripheral blood T cells into CD4+CD25+FoxP3+ regulatory T (Treg) cells. Intriguingly, the population of CD4+CD25+FoxP3+ Treg cells co-cultured with PD-MSCs was significantly expanded in comparison to those co-cultured with BM-MSCs or WI38 cells (p<0.05, p<0.001). Dynamic expression patterns of several cytokines, including anti- and pro-inflammatory cytokines and members of the transforming growth factor-beta (TGF-β) family secreted from PD-MSCs according to FoxP3 expression were observed. The results suggest that PD-MSCs have an immunomodulatory effect on T cells by regulating FoxP3 expression [118].

Amniotic Membrane MSC Induce Treg

human amniotic membrane (hAMSC) and their conditioned medium (CM-hAMSC) modulate lymphocyte proliferation in a dose-dependent manner. In order to understand the mechanisms involved in immune regulation exerted by hAMSC, we analyzed the effects of CM-hAMSC on T-cell polarization towards Th1, Th2, Th17, and T-regulatory (Treg) subsets. We show that CM-hAMSC equally suppresses the proliferation of both CD4(+) T-helper (Th) and CD8(+) cytotoxic T-lymphocytes. Moreover, we prove that the CM-hAMSC inhibitory ability affects both central (CD45RO(+)CD62L(+)) and effector memory (CD45RO(+)CD62L(−)) subsets. We evaluated the phenotype of CD4(+) cells in the MLR setting and showed that CM-hAMSC significantly reduced the expression of markers associated to the Th1 (T-bet(+)CD119(+)) and Th17 (RORγt(+)CD161(+)) populations, while having no effect on the Th2 population (GATA3(+)CD193(+)/GATA3(+)CD294(+) cells). T-cell subset modulation was substantiated through the analysis of cytokine release for 6 days during co-culture with alloreactive T-cells, whereby we observed a decrease in specific subset-related cytokines, such as a decrease in pro-inflammatory, Th1-related (TNFα, IFNγ, IL-1β), Th2 (IL-5, IL-6), Th9 (IL-9), and Th17 (IL-17A, IL-22). Furthermore, CM-hAMSC significantly induced the Treg compartment, as shown by an induction of proliferating CD4(+)FoxP3(+) cells, and an increase of CD25(+)FoxP3(+) and CD39(+)FoxP3(+) Treg in the CD4(+) population. Induction of Treg cells was corroborated by the increased secretion of TGF-β. Taken together, these data strengthen the findings regarding the immunomodulatory properties of CM-hAMSC derived from human amniotic membrane MSC, and in particular provide insights into their effect on regulation of T cell polarization [119].

iPS Generated MSC Induce Treg

human-induced pluripotent stem (huIPS) cells, remediating part of these issues, are considered as well as a valuable tool for therapeutic approaches, but their functions remained to be fully characterized. We generated multipotent MSCs derived from huiPS cells (huiPS-MSCs), and focusing on their immunosuppressive activity, we showed that human T-cell activation in coculture with huiPS-MSCs was significantly reduced. We also observed the generation of functional CD4+FoxP3+ regulatory T (Treg) cells. Further tested in vivo in a model of human T-cell expansion in immune-deficient NSG mice, huiPS-MSCs immunosuppressive activity prevented the circulation and the accumulation of activated human T cells. Intracytoplasmic labeling of cytokines produced by the recovered T cells showed reduced percentages of human-differentiated T cells producing Th1 inflammatory cytokines. By contrast, T cells producing IL-10 and FoxP3+-Treg cells, absent in non-treated animals, were detected in huiPS-MSCs treated mice [120].

Umbilical Cord Blood MSC stimulate Treg

MSCs were obtained from human umbilical cord blood and characterized according to their surface antigen expression and multilineage differentiation capacities. PBMCs isolated from SLE patients were divided into five groups, including sham, control, and three treatment groups. The treatment groups were treated by co-culturing MSCs to PBMCs with ratio of 1:1 (T1), 1:25 (T2), and 1:50 (T3) for 72 h incubation. The expression of CD4, CD25, and Foxp3 in Treg was analyzed by flow cytometry assay while TGFβ1 level was determined by Cytometric Bead Array (CBA). This study showed that the percentage of CD4+CD25+Foxp3+ iTreg cells was significantly increased in T1 and T2. This finding was aligned with the significant increase of TGFβ1 level. MSCs promote iTreg cells generation from human SLE PBMCs by releasing TGFβ1 to control SLE disease [121].

Embryonic Stem Cells Induce Treg

In one study, both MSC and ES types of stem cells suppressed not only the proliferation but also survival of CD4(+) T cells in vitro. They suppressed secretion of various cytokines (IL-2, IL-12, IFN-γ, TNF-α, IL-4, IL-5, IL-1β, and IL-10), whereas there was no change in the levels of TGF-β or IDO. Classic and modified transwell experiments demonstrated that immunosuppressive activities were mainly mediated by cell-to-cell contact. Granzyme B in the ESCs played a significant role in their immunosuppression, whereas PDL-1, Fas ligand, CD30 or perforin was not involved in the contact-dependent immunosuppression. However, none of the above molecules played a significant role in the immunosuppression by the MSCs. Interestingly, both stem cells increased the proportion of Foxp3(+) regulatory T cells [122].

Generation of Treg by MSC in Vivo

Search pubmed foxp3 and mesenchymal 409 come up

On page 303

Aplastic Anemia

A mean of 6×10(5)/kg (range, 5.0-7.1×10(5)) MSCs were injected intravenously to 18 patients, including 14 patients with nonsevere AA and four patients with severe AA who were refractory to prior immunosuppressive treatment. The outcomes of patients treated with MSCs were evaluated and compared with a historic control cohort, including 18 patients with refractory AA. Two patients had injection-related adverse events, including transient fever and headache. No major adverse events were reported during the follow-up period. An immunological analysis revealed an increased proportion of CD4(+)CD25(+) FOXP3(+) regulatory T cells in peripheral mononuclear cells. Following up for 1 year, six of 18 patients (33.3%) achieved a complete response or a partial response to MSC treatment. In six patients, two achieved a complete response including a recovery of three hematopoietic cell lines after MSCs therapy at days 88 and 92, two patients achieved only a red cell recovery with hemoglobin levels >100 g/L at days 30 and 48 and two patients had only a platelet recovery with a platelet count of >60×10(9)/L at days 54 and 81. In the control cohort, only one patient (5.56%) achieved a partial response during the follow-up period. The data from the present study suggest that treatment with MSCs from a related donor may be a promising therapeutic strategy for patients with refractory AA [123].

In this study, mesenchymal stromal cells (MSCs) obtained from bone marrow donors were concentrated and intravenously injected into 15 chronic AA patients who had been refractory to prior immunosuppressive therapy. We showed that BMMSCs modulate the levels of Th1, Th2, Th17 and Treg cells, as well as their related cytokines in chronic AA patients. Furthermore, the percentages of Th1 and Th17 cells among the H-MSCs decreased significantly, while the percentage Treg cells increased. The Notch/RBP-J/FOXP3/RORγt pathway was involved in modulating the Treg/Th17 balance after MSCs were transfused in vitro. Additionally, the role played by transfused MSCs in regulating the Treg/Th17 balance via the Notch/RBP-J/FOXP3/RORγt pathway was further confirmed in an AA mouse model. In summary, in humans with chronic AA, BMMSCs regulate the Treg/Th17 balance by affecting the Notch/RBP-J/FOXP3/RORγt pathway [124].

Transplantation

It was investigated whether mesenchymal stem cells (MSC) had immunomodulatory properties in solid organ allotransplantation, using a semiallogeneic heart transplant mouse model, and studied the mechanism(s) underlying MSC tolerogenic effects. Either single (portal vein, day −7) or double (portal vein, day −7 and tail vein, day −1) pretransplant infusions of donor-derived B6C3 MSC in B6 recipients induced a profound T cell hyporesponsiveness and prolonged B6C3 renal allograft survival. The protolerogenic effect was abrogated when donor-derived MSC were injected together with B6C3 hematopoietic stem cells (HSC), suggesting that HSC negatively impact MSC immunomodulatory properties. Both the induction (pretransplant) and the maintenance phase (>100 days posttransplant) of donor-derived MSC-induced tolerance were associated with CD4(+)CD25(+)Foxp3(+) Treg expansion and impaired anti-donor Th1 activity. MSC-induced regulatory T cells (Treg) were donor-specific since adoptive transfer of splenocytes from tolerant mice prevented the rejection of fully MHC-mismatched donor-specific secondary allografts but not of third-party grafts. In addition, infusion of recipient-derived B6 MSC tolerized a semiallogeneic B6C3 renal allograft, but not a fully MHC-mismatched BALB/c graft, and expanded Treg. A double i.v. pretransplant infusion of recipient-derived MSC had the same tolerogenic effect as the combined intraportal/i.v. MSC infusions, which makes the tolerogenic protocol applicable in a clinical setting. In contrast, single MSC infusions given either peritransplant or 1 day after transplant were less effective. Altogether these findings indicate that MSC immunomodulatory properties require HSC removal, partial sharing of MHC Ags between the donor and the recipient and pretransplant infusion, and are associated with expansion of donor-specific Treg [125].

It was investigated whether MSCs could prolong allograft survival. Treatment involving infusion of MSCs into BALB/c recipients 24 hours after receiving a heart allograft from a C57BL/6 donor significantly abated rejection and doubled graft mean survival time compared to untreated recipients. Furthermore, combination therapy of MSCs and low-dose Rapamycin (Rapa) achieved long-term heart graft survival (>100 days) with normal histology. The treated recipients readily accepted donor skin grafts but rejected third-party skin grafts, indicating the establishment of tolerance. Tolerant recipients exhibited neither intragraft nor circulating antidonor antibodies, but demonstrated significantly high frequencies of both tolerogenic dendritic cells (Tol-DCs) and CD4(+)CD25(+)Foxp3(+) T cells in the spleens. Infusion of GFP(+)C57BL/6-MSCs in combination with Rapa revealed that the GFP-MSCs accumulated in the lymphoid organs and grafts of tolerant recipients. Thus, engraftment of infused MSCs within the recipient's lymphoid organs and allograft appeared to be instrumental in the induction of allograft-specific tolerance when administered in combination with a subtherapeutic dose of Rapamycin. This study supports the clinical applicability of MSCs in transplantation [126].

A report described the immunoregulatory effects of rat MSCs in a model of allogeneic liver transplantation. Brown Norway rats received livers from inbred Lewis rats, and at designated intervals, infusions of MSCs derived from recipient, donor, or third-party rats. Allograft rejection and recipient survival rates were recorded. In particular, changes in circulating regulatory T cells (Tregs) were measured. After administration of MSCs derived from each of the 3 strains, allograft recipients demonstrated markedly longer survival compared with control animals. Histologic analysis revealed significant inhibition of allograft rejection. The MSCs induced generation of CD4+CD25+Foxp3+ Tregs. We concluded that MSCs inhibit acute rejection of allografts after liver transplantation, and propose that the immunoregulatory effects of MSCs are associated with expansion of Tregs [127].

In another study, MSCs (1×10(6), intravenously) from wild-type (WT-MSCs) or IDO knockout (IDO(−/−)-MSCs) C57BL/6 mice were injected into BALB/c recipients 24 hr after receiving a life-supporting orthotopic C57BL/6 renal graft. WT-MSC-treated recipients achieved allograft tolerance with normal histology and undetectable antidonor antibody levels. Tolerant recipients demonstrated increased circulating kynurenine levels and significantly high frequencies of tolerogenic dendritic cells. They also exhibited significantly impaired CD4+ T-cell responses consisting of decreased donor-specific proliferative ability and a Th2-dominant cytokine shift. In addition, high frequencies of CD4+CD25+Foxp3+ regulatory T cells (Tregs) were found in recipient spleens and donor grafts, with antibody-induced CD25+ cell depletion confirming the critical role of Tregs in the MSC-induced tolerance. Interestingly, renal allograft recipients treated with WT MSCs concomitant with the IDO inhibitor 1-methyl-tryptophan, or those treated with IDO(−/−)-MSCs alone, were unable to achieve allograft tolerance-revealing that functional IDO was necessary for the immunosuppression observed with WT-MSC treatment. It was found that IDO secreted by MSCs was responsible, at least in part, for induction of kidney allograft tolerance through generation of Tregs. This study supports the clinical application of MSCs in transplantation [128].

In islet transplantation, the therapeutic potential of autologous MSCs for preventing graft rejection was evaluated. The authors assessed the ability of MSCs to elicit an antiproliferative response in alloreactive lymphocytes and tested the immunosuppressive effect of MSCs in allogeneic islet transplantation. In islet allotransplantation, injection of autologous MSCs or a subtherapeutic dose of cyclosporine A (CsA; 5 mg/kg) alone did not prolong allograft survival. However, graft survival was attained for >100 d in 33% of autologous MSC-plus-CsA-treated recipients, indicating that graft acceptance was achieved in a subgroup of allograft recipients. Splenocytes from autologous MSC-plus-CsA-treated rats exhibited a reduced mixed lymphocyte reaction (MLR)-proliferative response to donor stimulators and increased interleukin (IL)-10 release. Interestingly, after excluding host CD11b(+) cells, splenic T cells from autologous MSC-plus-CsA-treated rats did not produce IL-10 or did not inhibit proliferative responses under the same conditions. The use of autologous MSC-plus-CsA downregulated immune responses, inducing donor-specific T-cell hyporesponsiveness by reducing the production of proinflammatory cytokines and inducing antiinflammatory cytokine production, especially that of IL-10, during the early posttransplantation period. T-regulatory cells made a contribution at a later phase. In conclusion, the combined use of autologous MSCs and low-dose CsA exerted a synergistic immunosuppressive effect in an islet allograft model, suggesting a role for autologous MSCs as an immune modulator [129].

Outbred miniature swine underwent hemi-facial allotransplantation (day 0). Group-I (n=5) consisted of untreated control animals. Group-II (n=3) animals received MSCs alone (given on days −1, +1, +3, +7, +14, and +21). Group-III (n=3) animals received CsA (days 0 to +28). Group-IV (n=5) animals received CsA (days 0 to +28) and MSCs (days −1, +1, +3, +7, +14, and +21). The transplanted face tissue was observed daily for signs of rejection. Biopsies of donor tissues and recipient blood sample were obtained at specified predetermined times (per 2 weeks post-transplant) or at the time of clinically evident rejection. Our results indicated that the MSC-CsA group had significantly prolonged allograft survival compared to the other groups (P<0.001). Histological examination of the MSC-CsA group displayed the lowest degree of rejection in alloskin and lymphoid gland tissues. TNF-α expression in circulating blood revealed significant suppression in the MSC and MSC-CsA treatment groups, as compared to that in controls. IHC staining showed CD45 and IL-6 expression were significantly decreased in MSC-CsA treatment groups compared to controls. The number of CD4+/CD25+ regulatory T-cells and IL-10 expressions in the circulating blood significantly increased in the MSC-CsA group compared to the other groups. IHC staining of alloskin tissue biopsies revealed a significant increase in the numbers of foxp3(+) T-cells and TGF-β1 positive cells in the MSC-CsA group compared to the other groups [130].

In another study, scientists examined in two living-related kidney transplant recipients whether: (i) pre-transplant (DAY-1) infusion of autologous MSC protected from the development of acute graft dysfunction previously reported in patients given MSC post-transplant, (ii) avoiding basiliximab in the induction regimen improved the MSC-induced Treg expansion previously reported with therapy including this anti-CD25-antibody. In patient 3, MSC treatment was uneventful and graft function remained normal during 1 year follow-up. In patient 4, acute cellular rejection occurred 2 weeks post-transplant. Both patients had excellent graft function at the last observation. Circulating memory CD8(+) T cells and donor-specific CD8(+) T-cell cytolytic response were reduced in MSC-treated patients, not in transplant controls not given MSC. CD4(+) FoxP3(+) Treg expansion was comparable in MSC-treated patients with or without basiliximab induction [131].

In another study, Recipient Sprague-Dawley rats were transplanted with hearts from Wistar rats. Wistar rat MSCs were infused via i.t. or i.v. or combined i.t. and i.v. (i.t./i.v.) injection at designated intervals. In vitro mixed lymphocyte reaction assays were performed to assess the immunosuppressive capacity of MSCs. Mesenchymal stem cell surface markers and CD4+, CD25+, and Foxp3+ T-cells in the peripheral blood were detected using flow cytometry analysis. The expression of microRNAs and cytokines in graft infiltrating lymphocytes was analyzed by real-time polymerase chain reaction. The MSCs cultured in vitro had multipotential differentiation capacity. Mixed lymphocyte reaction assays showed that donor-derived MSCs could not stimulate a proliferative response of recipient lymphocytes and could markedly suppress T-cell responses. Survival of the allografts was significantly prolonged by administration of i.t./i.v. injection of MSCs compared with controls, with a mean survival of 32.2 versus 6.5 d, respectively. Compared with the syngeneic groups posttransplant, miR-155 expression was significantly increased in the allogeneic group, and could be restored by injection of MSCs, especially i.t./i.v. injection of MSCs. Moreover, i.t./i.v. injection of MSCs decreased the level of interleukin (IL)-2 and interferon-gamma, but increased the levels of IL-4 and IL-10 in the allogeneic group. More important, i.t./i.v. injection of MSCs was the best way to increase the percentage of CD4+, CD25+, and Foxp3+ T-cell peripheral blood [132].

mmunomodulatory effects of regulatory T cells (Tregs) can aid the maintenance of immunoregulatory functions of MSCs, and that a combinatorial approach to cell therapy can have synergistic immunomodulatory effects on allograft rejection. After preconditioning with Fludarabine, followed by total body irradiation and anti-asialo-GM-1(ASGM-1), tail skin grafts from C57BL/6 (H-2k(b)) mice were grafted onto the lateral thoracic wall of BALB/c (H-2k(d)) mice. Group A mice (control group, n=9) did not receive any further treatment after preconditioning, whereas groups B and C (n=9) received cell therapy with MSCs or Tregs, respectively, on days −1, +6 and +13 relative to the skin transplantation. Group D (n=10) received cell therapy with MSCs and Tregs on days −1, +6 and +13. Cell suspensions were obtained from the spleens of five randomly chosen mice from each group on day +7, and the immunomodulatory effects of the cell therapy were evaluated by flow cytometry and real-time PCR. Our results show that allograft survival was significantly longer in group D compared to the control group (group A). Flow cytometric analysis and real-time PCR for splenocytes revealed that the Th2 subpopulation in group D increased significantly compared to the group B. Also, the expression of Foxp3 and STAT 5 increased significantly in group D compared to the conventional cell therapy groups (B and C). Taken together, these data suggest that a combined cell therapy approach with MSCs and Tregs has a synergistic effect on immunoregulatory function in vivo, and might provide a novel strategy for improving survival in allograft transplantation [133].

A murine heterotopic tracheal transplant model was used to study the continuum from acute to chronic rejection. In the treatment groups, PMSCs or PMSC-conditioned medium (PMSCCM) were injected either locally or intratracheally into the allograft. Phosphate-buffered saline (PBS) or blank medium was injected in the control groups. Tracheal luminal obliteration was assessed on sections stained with hematoxylin and eosin. Infiltration of inflammatory and immune cells and epithelial progenitor cells was assessed using immunohistochemistry and densitometric analysis. Compared with injection of PBS, local injection of PMSCs significantly reduced luminal obliteration at 28 days after transplantation (P=0.015). Intratracheal injection of PMSCs showed similar results to local injection of PMSCs compared with injection of PBS and blank medium (P=0.022). Tracheas treated with PMSC/PMSCCM showed protection against the loss of epithelium on day 14, with an increase in P63+CK14+ epithelial progenitor cells and Foxp3+ regulatory T cells. In addition, injection of PMSCs and PMSCCM significantly reduced the number of neutrophils and CD3+ T cells on day 14 [134].

Syngeneic MSCs transduced with IL-10 were delivered via the right jugular vein 30 min post-orthotopic transplantation in the rat model. To evaluate liver morphology and measure cytokine concentration, the blood and liver samples from each animal group were collected at different time-points (3, 5 and 7 days) post-transplantation. The mean survival time of the rats treated with MSCs-IL-10 was shown to be much longer than those treated with saline. According to Banff scheme grading, the saline group scores increased significantly compared with those in the MSCs-IL-10 group. Retinoid acid receptor-related orphan receptor gamma t (RORγt) expression was more increased in the saline group compared to those in the MSCs-IL-10 group in a time-dependent manner; forkhead box protein 3 (FoxP3) expression also decreased significantly in the saline group compared with those in the MSCs-IL-10 group in a time-dependent manner. The expression of cytokines [IL-17, IL-23, IL-6, interferon (IFN)-γ and tumour necrosis factor (TNF)-α] in the saline groups increased significantly compared with the time-point-matched MSCs-IL-10 group, whereas cytokine expression of (IL-10, TGF-β1) was deceased markedly compared to that in the MSCs-IL-10 group. These results suggest a potential role for IL-10-engineered MSC therapy to overcome clinical liver transplantation rejection [135].

The anti-alloimmunity of donor MSCs in the presence or absence of RAPA was examined in both mouse renal allograft model (C57BL/6 to BALB/c mice) and a variety of cultured immune cells. Immunohistochemical staining was used for the measurement of intragraft antibody deposition, and fluorescence-activated cell sorting (FACS) for the determination of serum alloantibodies and leukocyte phenotypes: B7-H1 expression in cultured MSCs was up-regulated following IFN-γ stimulation. In transplant recipients, combination therapy of MSCs and RAPA induced immune tolerance to allografts, but blockade of B7-H1 on MSCs with monoclonal antibody abrogated the combination therapy-induced immune tolerance as heart allografts were rejected. The negative effect of MSC-expressing B7-H1 neutralization on graft survival was correlated with a reduction of regulatory immune cells (CD4(+)CD25(+)Foxp3(+) T cells, tolerogenic dendritic cells and IL-4(high)IL-10(High)CD83(low) B cells), and also with an increase in alloantibody (IgG and IgM) levels both inside the grafts and in the circulation as compared with un-neutralized controls. In vitro MSC-mediated suppression of antibody production and B cell proliferation depended on B7-H1 function and cell contact between CD19(+) B cells and MSCs. These data suggest that MSC-expressing B7-H1 mediates the immune tolerance to renal allografts in recipients receiving MSC and RAPA combination therapy [136].

A study was conducted to assesses the safety and feasibility of autologous mesenchymal stromal cell (MSC) transplantation in four patients that underwent living donor renal transplantation, and the effect on the immunophenotype and functionality of peripheral T lymphocytes following transplantation: All patients received low dose ATG induction followed by calcineurin inhibitor-based triple drug maintenance immunosuppression. Autologous MSCs were administered intravenously pre transplant and day 30 post-transplant. Patients were followed up for 6 months. The frequency of regulatory T cells and T cell proliferation was assessed at different time points. None of the four patients developed any immediate or delayed adverse effects following MSC infusion. All had excellent graft function, and none developed graft dysfunction. Protocol biopsies at 1 and 3 months did not reveal any abnormality. Compared to baseline, there was an increase in the CD4+CD25+FOXP3+ regulatory T cells and reduction in CD4 T cell proliferation. The authors concluded that autologous MSCs can be used safely in patients undergoing living donor renal transplantation, lead to expansion of regulatory T cells and decrease in T cell proliferation. Larger randomized trials studies are needed to confirm these findings and evaluate whether this will have any impact on immunosuppressive therapy [137].

In series of experiments MSC were injected into the renal artery soon after reperfusion. Controls were grafted untreated and normal rats. Rats were sacrificed 7 days after grafting. Serum and renal tissue levels of IFN-γ, IL-1, IL-2, IL-4, IL-6, IL-10, MSP/RON, HGF/Met systems, Treg lymphocytes were investigated. In grafted untreated rats IFN-γ increased in serum and renal tissue and IL-6 rose in serum. MSC prevented both the phenomena, increased IL-10 serum levels and Treg number in the graft. Furthermore MSC increased serum and tissue HGF levels, Met tubular expression and prevented the suppression of tubular MSP/RON expression. The results demonstrate that MSC modify cytokine network to a tolerogenic setting, they suppress Th1 cells, inactivate monocytes/macrophage, recruit Tregs. In addition, MSC sustain the expression of the Scatter Factor systems expression, i.e. systems that are committed to defend survival and stimulate regeneration of tubular cells [138].

The ability of MSCs from three distinct sources to prolong rat corneal allograft survival. A fully allogeneic rat cornea transplant model (DA to LEW) was used. Recipient rats received 1×10(6) MSCs (syn [LEW], allo [DA] or third-party [Wistar Furth]) intravenously 7 days before transplantation and again on the day of transplantation (day 0). A high percentage of untreated and syn-MSC treated allografts were rejected (80% and 100%, respectively). Preactivation of syn-MSCs with interferon gamma also failed to prolong allograft survival. Conversely, corneal allograft survival was significantly prolonged in allo-MSC treated (90%) and third-party MSC treated (80%) allograft recipients. Flow cytometric analysis revealed less infiltrating natural killer T cells in corneas of both allo- and third-party MSC treated animals, coupled with a higher proportion of splenic CD4+Foxp3+ regulatory T cells, compared to controls. In the case of allo- and third-party MSCs, results from a delayed-type hypersensitivity assay clearly showed that hypo-responsiveness was specific for corneal donor-associated allo-antigens [139].

MSCs from C57BL/6 (H2b) mice were infused together with fully major histocompatibility complex-mismatched Balb/c (H2d) allogeneic islets into the portal vein of diabetic C57BL/6 (H2b) mice, which were subsequently treated with costimulation blockade for the first 10 days after transplantation. Mice receiving both recipient-type MSCs, CTLA4Ig, and anti-CD40L demonstrated indefinite graft acceptance, just as did most of the recipients receiving MSCs and CTLA4Ig. Recipients of MSCs only rejected their grafts, and fewer than one half of the recipients treated with costimulation blockade alone achieved permanent engraftment. The livers of the recipients treated with MSCs plus costimulation blockade contained large numbers of islets surrounded by Foxp3+ regulatory T cells. These recipients showed reduced antidonor IgG levels and a glucose tolerance similar to that of naïve nondiabetic mice. Intrahepatic lymphocytes and splenocytes from these recipients displayed reduced proliferation and interferon-γ production when re-exposed to donor antigen. MSCs in the presence of costimulation blockade prevented dendritic cell maturation, inhibited T cell proliferation, increased Foxp3+ regulatory T cell numbers, and increased indoleamine 2,3-dioxygenase activity. These results indicate that MSC infusion and costimulation blockade have complementary immune-modulating effects that can be used for a broad number of applications in transplantation, autoimmunity, and regenerative medicine [140].

utilized a lentivirus vector to overexpress the therapeutic gene Foxp3 on MSC. In vivo, Injections of 2×10(6) MSC, FUGW-MSC or Foxp3-MSC into the portal vein were carried out immediately after liver transplantation. Successful gene transfer of Foxp3 in MSC was achieved by lentivirus carrying Foxp3 and Foxp3-MSC engraftment in liver allograft was confirmed by fluorescence microscopy. Foxp3-MSC treatment significantly inhibited the proliferation of allogeneic ACI CD4(+) T cells to splenocytes (SC) from the same donor strain or third-party BN rat compared with MSC. Foxp3-MSC suppressive effect on the proliferation of CD4(+) T cells is contact dependent and associated with Programmed death ligand 1(PD-L1) upregulation in MSC. Co-culture of CD4(+) T cells with Foxp3-MSC results in a shift towards a Tregs phenotype. More importantly, Foxp3-MSC monotherapy achieved donor-specific liver allograft tolerance and generated a state of CD4(+)CD25(+)Foxp3(+) Tregs-dependent tolerance. Foxp3-engineered MSC therapy seems to be a promising and attractive cell therapy approach for inducing immunosuppression or transplant tolerance [141].

Wild-type (WT) MSCs, empty lentivirus-transfected MSCs (Lenti-MSCs) or IDO-lentivirus-transfected MSCs (IDO-MSCs) were cocultured with peripheral blood mononuclear cells (PBMCs) or CD4CD25 regulatory T (Treg) cells to examine the impact of IDO on the immunoregulatory properties of MSCs in vitro. WT-MSCs, Lenti-MSCs or IDO-MSCs (2×10/kg) were intravenously injected into rabbit renal transplant recipients immediately after surgery to examine the role of IDO-MSCs in tolerance induction in vivo. Lentivirus infection of MSCs resulted in stable expression of IDO. The IDO-MSCs inhibited the proliferation of CD4CD25 effector T cells to a greater extent than WT-MSCs. Coculture of PBMCs and IDO-MSCs induced a higher percentage of CD4CD25Foxp3 Treg cells in PBMCs. Additionally, the antigen-specific suppressive function of these CD4CD25 Treg cells was increased. The IDO-MSCs-treated Treg cells showed upregulated expression of cytotoxic T-lymphocyte-associated antigen 4 and increased secretion of IL-10 and TGF-β. Low doses of IDO-MSCs prolonged graft survival and induced tolerance by inducing antigen-specific CD4CD25 Treg cells, as evidenced by the finding that IDO-MSCs-treated kidney transplant recipients accepted donor-specific skin grafts but rejected third-party grafts. The IDO increased the direct immunoregulatory properties of MSCs. The IDO-MSCs enhanced the expression and function of CD4CD25 Foxp3 Treg cells and induced allograft tolerance [142].

investigated whether co-transplantation of MSCs could improve the survival of other transplanted therapeutic cells. Allogeneic glial-restricted precursors (GRPs) were isolated from the brain of a firefly luciferase transgenic FVB mouse (at E13.5 stage) and intracerebrally transplanted, either alone, or together with syngeneic MSCs in immunocompetent BALB/c mice (n=20) or immunodeficient Rag2(−/−) mice as survival control (n=8). No immunosuppressive drug was given to any animal. Using bioluminescence imaging (BLI) as a non-invasive readout of cell survival, we found that co-transplantation of MSCs significantly improved (p<0.05) engrafted GRP survival. No significant change in signal intensities was observed in immunodeficient Rag2(−/−) mice, with transplanted cells surviving in both the GRP only and the GRP+MSC group. In contrast, on day 21 post-transplantation, we observed a 94.2% decrease in BLI signal intensity in immunocompetent mice transplanted with GRPs alone versus 68.1% in immunocompetent mice co-transplanted with MSCs and GRPs (p<0.05). Immunohistochemical analysis demonstrated a lower number of infiltrating CD45, CD11b(+) and CD8(+) cells, reduced astrogliosis, and a higher number of FoxP3(+) cells at the site of transplantation for the immunocompetent mice receiving MSCs [143].

Another study examined the therapeutic potential of transforming growth factor (TGF-β)-overexpressing mesenchymal stem cells (MSCs) in inducing a local immunosuppression in liver grafts after transplantation. MSCs were transduced with a lentiviral vector expressing the human TGF-β1 gene; TGF-β1-overexpressing MSCs (designated as TGF/MSCs) were then transfused into the liver grafts via the portal vein of a rat LT model of acute rejection. Rejection severity was assessed by clinical and histologic analysis. The immunity suppression effects and mechanism of TGF/MSCs were tested, focusing on their ability to induce generation of regulatory T cells (Tregs) in the liver grafts. Our findings demonstrate that transfusion of TGF/MSCs prevented rejection, reduced mortality, and improved survival of rats after LT. The therapeutic effects were associated with the immunosuppressive effects of MSCs and TGF-β1. Their reciprocal effects on Tregs induction and function resulted in more CD4+Foxp3+Helios-induced Tregs, fewer Th17 cells, and improved immunosuppressive effects in local liver grafts. Thus, TGF/MSCs can induce a local immunosuppressive effect in liver grafts after transplantation [144].

Another study aimed to investigate OX40-Ig fusion protein (OX40Ig) expression in ADSCs and to validate their more potent immunosuppressive activity in preventing renal allograft rejection. For this purpose, ADSCs from Lewis rats were transfected with the recombinant plasmid, pcDNA3.1(−)OX40Ig, by nucleofection. The ADSCs transduced with the plasmid (termed ADSCsOX40Ig) or untransduced ADSCs (termed ADSCsnative) were added to allostimulated mixed lymphocyte reaction (MLR) in vitro. In vivo, ADSCsOX40Ig, ADSCsnative, or PBS were administered to an allogeneic renal transplantation model, and the therapeutic effects, as well as the underlying mechanisms were examined. The results revealed that both the ADSCsnative and ADSCsOX40Ig significantly suppressed T cell proliferation and increased the percentage of CD4+CD25+ regulatory T cells in allogeneic MLR assays, with the ADSCsOX40Ig being more effective. Furthermore, the results from our in vivo experiments revealed that compared with the ADSCsnative or PBS group, the administration of autologous ADSCsOX40Ig markedly prolonged the mean survival time of renal grafts, reduced allograft rejection, and significantly downregulated the mRNA expression of intragraft interferon-γ (IFN-γ), and upregulated the mRNA expression of interleukin (IL)-10, transforming growth factor-β (TGF-β) and forkhead box protein 3 (Foxp3). The findings of our study indicate that the use of ADSCsOX40Ig is a promising strategy for preventing renal allograft rejection. This strategy provides the synergistic benefits of ADSC immune modulation and OX40-OX40L pathway blockade, and may therefore have therapeutic potential in clinical renal transplantation [145].

his study investigated the efficacy of recipient autologous adipose-derived stem cells (rADSCs) for VCA survival. The heterotopic hind-limb transplantation from female donor to male recipient was performed in outbred miniature swine. Group I (n=6) was untreated controls. Group II (n=4) obtained rADSCs infusions (given on weeks 0, +1, +2, and +3). Group III (n=4) obtained tacrolimus (FK506, weeks 0 to +4). Group IV (n=8) received irradiation (IR; day −1), FK506 (weeks 0 to +4), and rADSC infusions (weeks 0, +1, +2, and +3). The results revealed treatment with multiple injections of rADSCs along with IR and FK506 resulted in a statistically significant increase in allograft survival. The percentage of CD4+/CD25+/Foxp3+ regulatory T cells were significantly increased in the rADSC-IR-FK506 group as compared to controls. Analysis of recipient peripheral blood revealed that transforming growth factor β1 (TGFβ1) was significantly increased in the rADSC-IR-FK506 group. The polymerase chain reaction (PCR) analysis and immunohistochemical staining showed recipient sex-determining region of Y (SRY) chromosome gene expression existed in donor allotissues in the rADSC-IR-FK506 group. These results indicate that rADSCs in addition to IR and transient immunosuppressant could prolong allotransplant survival, modulate T-cell regulation, and enhance recipient cell engraftment into the allotransplant tissues [146].

The present study was undertaken to investigate the efficacy of ERCs in inducing renal allograft tolerance and the function of stromal cell-derived factor-1 (SDF-1) in the ERC-mediated immunoregulation. The inhibitory efficacy of human ERCs in the presence or absence of rapamycin was examined in both mouse renal allograft models between BALB/c (H-2d) donors and C57BL/6 (H-2b) recipients and in vitro cocultured splenocytes. AMD3100 was used to inhibit the function of SDF-1. Intragraft antibody (IgG and IgM) deposition and immune cell (CD4+ and CD8+) infiltration were measured by immunohistochemical staining, and splenocyte phenotypes were determined by fluorescence-activated cell sorting analysis. The results showed that ERC-based therapy induced donor-specific allograft tolerance, and functionally inhibiting SDF-1 resulted in severe allograft rejection. The negative effects of inhibiting SDF-1 on allograft survival were correlated with increased levels of intragraft antibodies and infiltrating immune cells, and also with reduced levels of regulatory immune cells including MHC class IIlow CD86low CD40low dendritic cells, CD68+CD206+ macrophages, CD4+CD25+Foxp3+ T cells, and CD1dhigh CD5high CD83low IL-10high B cells both in vivo and in vitro. These data showed that human ERC-based therapy induces renal allograft tolerance in mice, which is associated with SDF-1 activity, suggesting that SDF-1 mediates the immunosuppression of ERC-based therapy for the induction of transplant tolerance [147].

examine the effects of BMSCs on immune tolerance of allogeneic heart transplantation and the involvement of CD45RB+ dendritic cells (DCs). Bone marrow-derived DCs and BMSCs were co-cultured, with CD45RB expression on the surface of DCs measured by flow cytometry. qRT-PCR and Western blotting were used to detect mRNA and protein levels. Cytometric bead array was performed to determine the serum level of IL-10. Survival time of transplanted heart and expression of CD4+, CD8+, IL-2, IL-4, IL-10, IFN-γ were determined. Immunofluorescence assay was employed to determine intensity of C3d and C4d. DCs co-cultured with BMSCs showed increased CD45RB and Foxp3 levels. CD45RB+ DCs co-cultured with T-cells CD4+ displayed increased T-cell CD4+ Foxp3 ratio and IL-10 than DCs. Both of them extended survival time of transplanted heart, decreased histopathological classification and score, intensity of C3d, C4d, proportion of CD4+, expression levels of IL-2 and IFN-γ, and increased the CD4+ Foxp3 ratio and levels of IL-4 and IL-10. CD45RB+ DCs achieved better protective effects than DCs. BMSCs increased the expression of CD45RB in the bone marrow-derived DCs, thereby strengthening immunosuppression capacity of T cells and immune tolerance of allogeneic heart transplantation [148].

study was to investigate the requirement of B7-H1 in the immunoregulation of ERCs in preventing transplant vasculopathy of aorta allografts. The results showed that B7-H1 expression on ERCs was upregulated by IFN-γ in a dose-dependent manner and it was required for ERCs to inhibit the proliferation of peripheral blood mononuclear cells (PBMCs) in vitro. ERCs could alleviate transplant vasculopathy, as the intimal growth of transplanted aorta was limited, and the preventive effects were correlated with an increase in the percentages of CD11c+MHC class IllowCD86low dendritic cells, CD68+CD206+ macrophages, and CD4+CD25+Foxp3+ T cells, as well as a decrease in the percentages of CD68+ macrophages, CD3+CD4+ T cells, CD3+CD8+ T cells, and donor-reactive IgM and IgG antibodies. Moreover, overexpression of B7-H1 by IFN-γ can promote the immunosuppressive effect of ERCs. These results suggest that overexpression of B7-H1 stimulated by IFN-γ is required for ERCs to prevent the transplant vasculopathy, and this study provides a theoretical basis for the future clinical use of human ERCs [149].

One study used Lewis rats as donors and ACI rats as recipients. Hematoxylin and eosin staining was performed to evaluate histomorphological changes, and Western blot was performed to measure protein expression. The expression of TGF-β1 in the liver allografts and spleen and protein levels of forkhead box P3 (FoxP3), interleukin-10 (IL-10), and cytotoxic T lymphocyte-associated antigen-4 (CTLA-4) were measured using Western blot. The suppressive capacity of CD4+CD25+ regulatory T cells was evaluated using the MTT assay. Cell-mediated immunotoxicity was evaluated using the mixed lymphocyte reaction of CD4+ T cells and cytotoxic T lymphocyte (CTL) assay of CD8+ T cells. The results showed that MSCs prolonged the survival of the OLT mice by regulating the expression of TGF-β1 at different time points. The administration of MSCs promoted a prolonged survival in the ACI recipients (105±6.6 d) compared with the MSC-untreated recipients (16.2±4.0 d). On the postoperative day (POD) 7, the MSC-treated recipients showed a significantly higher expression of TGF-β1, FoxP3, IL-10, and CTLA-4 than the MSC-untreated recipients. However, on POD 100, the MSC-treated recipients showed a lower expression of TGF-β1 and FOxP3 than that on POD 7. Moreover, on POD 7, CD4+CD25+ regulatory T cells extracted from the MSC-treated recipients showed a higher expression of FoxP3, IL-10, CTLA-4, and suppressive capacity. On POD 7, CD4+ T cells from the MSC-treated recipients showed more significantly diminished proliferative functions than the MSC-untreated recipients; further, a reduced allospecific CTL activity of CD8+ T cells was observed in the MSC-treated recipients [150].

Third-party (non-donor, non-recipient strain) allogeneic mesenchymal stromal cells (allo-MSC) were administered intravenously 7 and 1 days prior to cornea transplantation. Rejection-free graft survival to 30 days post-transplant improved from 0 to 63.6% in MSC-treated compared to vehicle-treated control animals (p=<0.0001). Pre-sensitized animals that received third-party allo-MSC prior to transplantation had significantly higher proportions of CD45+CD11b+B220+ monocytes in the lungs 24 h after the second MSC injection and significantly higher proportions of CD4+FoxP3+ regulatory T cells in the graft-draining lymph nodes at the average day of rejection of control animals. In in vitro experiments, third-party allo-MSC polarized primary lung-derived CD11b/c+ myeloid cells to a more anti-inflammatory phenotype, as determined by cytokine profile and conferred them with the capacity to suppress T cell activation via prostaglandin E2 and TGFβ1 [151].

GVHD

A GvHD mouse model was established by transplanting C3H/he donor bone marrow (BM) cells and spleen cells into lethally irradiated BALB/c recipient mice. MSC were obtained from C3H/he mice and the C3H/10T1/2 murine MSC line. The mRNA expression of Foxp3 in regional lymph nodes (LN) localized with T cells was markedly increased by the addition of C3H10T1/2 cells in a real-time polymerase chain reaction (PCR). Using a mixed lymphocyte reaction, we determined the optimal splenocyte proliferation inhibition dose (MSC:splenocyte ratios 1:2 and 1:1). Three different C3H10T1/2 cell doses (low, 0.5×10(6), intermediate, 1×10(6), and high, 2×10(6)) with a consistent splenocyte dose (1×10(6)) were evaluated for their therapeutic potential in an in vivo GvHD model. The clinical and histologic GvHD score and Kaplan-Meier survival rate were improved after MSC transplantation, and these results demonstrated a dose-dependent inhibition [152].

In this study, when a treatment with donor- or recipient-derived MSCs was administered from day 8 to day 14 after the typical symptoms of LT-aGVHD started, the recipients were not cured, and their survival time was not prolonged. However, when MSCs of different origins were administered from day 0 to day 6 after LT, the recipients survived significantly longer than the control group, and the surviving MSC-treated rats did not show typical LT-aGVHD symptoms. In vivo tracings of carboxyfluorescein diacetate succinimidyl ester-stained MSCs did not show significant accumulations in the target organs after administration. Flow cytometry analysis showed that the Treg ratios in peripheral blood were more higher for the MSC-treated groups versus the control group. More immunohistochemically stained forkhead box P3-positive cells were also found in the intestines of the MSC-treated groups versus the control group. Further investigations of the function of MSCs showed that they could increase the Treg ratio in a mixed lymphocyte reaction (MLR) and lead to a greater reduction in MLR proliferation in vitro. In conclusion, the post-LT administration of MSCs of either donor or recipient origin could prevent the onset of LT-aGVHD in our rat model [153].

A study was performed to investigate whether the immunomodulatory capacity of MSCs could be enhanced by combination infusion of regulatory T (Treg) cells to prevent acute GVHD (aGVHD) following MHC-mismatched bone marrow transplantation (BMT). For GVHD induction, lethally irradiated BALB/c (H-2(d)) mice were transplanted with bone marrow cells (BMCs) and spleen cells of C57BL/6 (H-2(b)) mice. Recipients were injected with cultured recipient-derived MSCs, Treg cells, or MSCs plus Treg cells (BMT+day 0, 4). Systemic infusion of MSCs plus Treg cells improved clinicopathological manifestations and survival in the aGVHD model. Culture of MSCs plus Treg cells increased the population of Foxp3(+) Treg cells and suppressed alloreactive T-cell proliferation in vitro. These therapeutic effects were associated with more rapid expansion of donor-type CD4(+)CD25(+)Foxp3(+) Treg cells and CD4(+)IL-4(+) type 2 T-helper (Th2) cells in the early posttransplant period. Furthermore, MSCs plus Treg cells regulated CD4(+)IL-17(+) Th17 cells, as well as CD4(+)IFN-γ(+) Th1 cells. These data suggest that the combination therapy with MSCs plus Treg cells may have cooperative effects in enhancing the immunomodulatory activity of MSCs and Treg cells in aGVHD [154].

This study evaluated the immunomodulation effects of mesenchymal stromal cells (MSCs) from bone marrow of a third-party donor for refractory aGVHD. Forty-seven patients with refractory aGVHD were enrolled: 28 patients receiving MSC and 19 patients without MSC treatment. MSCs were given at a median dose of 1×10(6) cells/kg weekly until patients got complete response or received 8 doses of MSCs. After 125 doses of MSCs were administered, with a median of 4 doses (range, 2 to 8) per patient, overall response rate was 75% in the MSC group compared with 42.1% in the non-MSC group (P=0.023). The incidence of cytomegalovirus, Epstein-Barr virus infections, and tumor relapse was not different between the 2 groups during aGVHD treatment and follow-up. The incidence and severity of chronic GVHD in the MSC group were lower than those in the non-MSC group (P=0.045 and P=0.005). The ratio of CD3(+)CD4(+)/CD3(+)CD8(+) T cells, the frequencies of CD4(+)CD25(+)Foxp3(+) regulatory T cells (Tregs), and the levels of signal joint T cell-receptor excision DNA circles (sjTRECs) after MSCs treatment were higher than those pretreatment. MSC-treated patients exhibited higher Tregs frequencies and sjTRECs levels than those in the non-MSC group at 8 and 12 weeks after treatment. MSCs derived from bone marrow of a third-party donor are effective to refractory aGVHD. It might reduce the incidence and severity of chronic GVHD in aGVHD patients by improving thymic function and induction of Tregs but not increase the risks of infections and tumor relapse [155].

Another study demonstrated that enhanced expression of HO-1 in target organs by infusing HO-1-gene-modified Mesenchymal stem cells (MSCs) alleviated the clinical and histopathological severity of aGVHD in experimental mice. Flow cytometry revealed a higher expression of Treg cells and a lower expression of TH17 cells in splenic and lymph node tissues of mice with enhanced HO-1 expression, as compared to that in the aGVHD mice. This was further substantiated by lower expression levels of ROR-Yt and IL-17A mRNA, and higher levels of Foxp3 mRNA in the splenic tissue of mice with enhanced HO-1 expression. Our results indicate that high expression of HO-1 may reduce the severity of aGVHD by regulation of the TH17/Treg balance [156].

Researchers showed that human gingival mesenchymal stem cells (GMSCs) can prevent and treat acute graft-versus-host disease (GVHD) in two different mouse models. Our results indicate that besides exhibiting suppressive function in vitro and in vivo, GMSCs may also regulate the conversion of Tregs to Th1 and/or Th17-like cells, as well as stabilize Foxp3 expression. Furthermore, GMSC-mediated prevention of acute GVHD was dependent on CD39 signaling that play an important role in the function and stability of Tregs. Finally, we also observed stronger protective ability of GMSCs with greater expansion ability compared with BMSCs or ASCs [157].

Investigators isolated and cultured BMSCs and Tregs. Then, we examined effects of miR-21 knockdown or overexpression and EGF on cell activities of BMSCs and the expression of PTEN, Foxp3, AKT phosphorylation, and extent of c-jun phosphorylation by gain- and loss-of-function approaches. The results showed that miR-21 promoted the proliferation, invasion, and migration of BMSCs. Furthermore, miR-21 in BMSCs-derived exosomes inhibited PTEN, but enhanced AKT phosphorylation and Foxp3 expression in Tregs. In addition, EGF enhanced c-jun phosphorylation to elevate the miR-21 expression. Furthermore, EGF significantly increased the efficacy of BMSCs in a mouse model of aGVHD, manifesting in reduced IFN-γ expression and lesser organ damage. Moreover, EGF treatment promoted the Foxp3 expression of Tregs in BMSCs-treated aGVHD mice [158].

Allergy

he influence of allogeneic MSC was examined in a model system where T(reg) induction is essential to prevent pathology. This was tested using a combination of a model of ovalbumin-driven inflammation with allogeneic MSC cell therapy. Systemic administration of allogeneic MSC protected the airways from allergen-induced pathology, reducing airway inflammation and allergen-specific IgE. MSC were not globally suppressive but induced CD4(+) FoxP3(+) T cells and modulated cell-mediated responses at a local and systemic level, decreasing IL-4 but increasing IL-10 in bronchial fluid and from allergen re-stimulated splenocytes. Moderate dose cyclophosphamide protocols were used to differentially ablate T(reg) responses; under these conditions the major beneficial effect of MSC therapy was lost, suggesting induction of T(reg) as the key mechanism of action by MSC in this model. In spite of the elimination of T(reg), a significant reduction in airway eosinophilia persisted in those treated with MSC. These data demonstrate that MSC induce T(reg) in vivo and reduce allergen-driven pathology. Multiple T(reg) dependent and independent mechanisms of therapeutic action are employed by MSC [159].

In another study, Peripheral blood mononuclear cells isolated from asthmatic patients and healthy controls were co-cultured with human bone marrow mesenchymal stem cells which were pretreated with Hemin (the revulsive of Heme Oxygenase-1), Protoporphyrin IX zinc (the inhibitor of Heme Oxygenase-1) and saline. The expression of Heme Oxygenase-1 in MSCs was enhanced by Hemin and inhibited by Protoporphyrin zinc in vitro. Overexpression of Heme Oxygenase-1 elevated the proportion of CD4+CD25+CD127low/− regulatory T cells in CD4+ T cells, meanwhile, inhibition of Heme Oxygenase-1 decreased the proportion of CD4+CD25+CD127low/− regulatory T cells in CD4+ T cells as compared with mesenchymal stem cells alone. Taken together, these data demonstrated that Heme Oxygenase-1 contributed to the up-regulation of CD4+CD25+CD127low/− regulatory T cells mediated by mesenchymal stem cells in asthma [160].

BALB/c mice were sensitized with OVA on days 0, 7, and 14, followed by 8-week OVA challenge from day 22. ADMSCs were injected via tail vein on day 21. Animals were measured for airway responsiveness, lung pathology, IgE and cytokine levels in serum, cell composition in bronchoalveolar lavage fluid (BALF), gene expression in the lung, and regulatory T cells (Tregs). We found that delivery of ADMSCs decreased airway responsiveness and eosinophil counts in BALF and reduced infiltration of inflammatory cells and number of mucus-expressing goblet cells in the lung in OVA-challenged mice. OVA-evoked elevation of serum IgE levels and alteration of cytokine production in serum and BALF was significantly prevented by ADMSCs. In addition, administration of ADMSCs impaired the regulation of lung IL-10, Foxp3, IL-17, and RORγ expression by OVA challenge and restored the percentage of CD4+CD25+Foxp3+ Tregs in the spleen. In conclusion, ADMSCs confer protection against OVA-induced airway hyperresponsiveness and inflammation, which is associated with induction of Tregs and restoration of immune homeostasis. These findings suggest that ADMSCs may have therapeutic implications for allergic asthma [161].

study was to assess the therapeutic effects of human placenta MSCs (hPMSCs) in asthma, and explore the underlying mechanisms; in addition, the impact of hPMSCs transplantation on Th17/Treg balance in lymph and serum samples from asthmatic animals was evaluated. Sprague-Dawley rats were sensitized and challenged with ovalbumin (OVA). Administration of hPMSCs from human placenta resulted in increased Th17 and Treg in lymph samples compared with peripheral blood specimens. Enhanced pause values in OVA-treated animals were significantly higher than those in the control and hPMSCs treatment groups. The numbers of total cells, macrophages, neutrophils, and eosinophils were markedly increased in the OVA group compared with those of control+hPMSCs and control groups. In addition, interleukin 10, forkhead box P3 (Foxp3) and Treg levels in lymph, peripheral blood and lung tissue samples from asthma rats were increased significantly following hPMSC transplantation. Furthermore, Foxp3 protein levels increased, while those of RAR-related orphan receptor γ (RORγt) decreased after hPMSCs transplantation compared with the asthma group. Reduced IL-17, RORγt and Th17 levels were accompanied by reduced inflammatory cell infiltration, sub-epithelial smooth layer attenuation and mucus production in lung tissues. These results suggest that hPMSCs may improve airway hyperresponsiveness and inflammation by regulating the Th17/Treg balance in rats with asthma [162].

Murine ASCs (mASCs) were isolated from male Balb/c mice and identified by the expression of surface markers using flow cytometry. The OVA-sensitized asthmatic mouse model was established and then animals were treated with the mASCs through intratracheal delivery. The therapy effects were assessed by measuring airway responsiveness, performing immuohistochemical analysis, and examining bronchoalveolar lavage fluid (BALF). Additionally, the expression of inflammatory cytokines and IgE was detected by CHIP and ELISA, respectively. The mRNA levels of serum indices were detected using qRT-PCR. The mASCs grew by adherence with fibroblast-like morphology, and showed the positive expression of CD90, CD44, and CD29 as well as the negative expression of CD45 and CD34, indicating that the mASCs were successfully isolated. Administering mASCs to asthmatic model animals through intratracheal delivery reduced airway responsiveness, the number of lymphocytes (P<0.01) and the expression of IgE (P<0.01), IL-1β (P<0.05), IL-4 (P<0.001), and IL-17F (P<0.001), as well as increased the serum levels of IL-10 and Foxp3, and the percentage of CD4+CD25+Foxp3+ Tregs in the spleen, and reduced the expression of IL-17 (P<0.05) and RORγ. Intratracheal administration of mASCs alleviated airway inflammation, improved airway remodeling, and relieved airway hyperresponsiveness in an OVA-sensitized asthma model, which might be associated with the restoration of Th1/Th2 cell balance by mASCs.[163]

Diabetes

MSC were CD45(−)/CD44(+)/CD54(+)/CD90(+)/CD106(+). MSC spontaneously secreted IL-6, HGF, TGF-beta1 and expressed high levels of SDF-1 and low levels of VEGF, IL-1beta and PGE(2), but no EGF, insulin or glucagon. MSC homed to the pancreas and this therapy allowed for enhanced insulin secretion and sustained normoglycemia. Interestingly, immunohistochemistry demonstrated that, the islets from MSC-treated rats expressed high levels of PDX-1 and that these cells were also positive for insulin staining. In addition, peripheral T cells from MSC-treated rats exhibited a shift toward IL-10/IL-13 production and higher frequencies of CD4(+)/CD8(+) Foxp3(+) T cells compared to the PBS-treated rats [164].

Prevention of spontaneous insulitis or of diabetes was evaluated after a single i.v. injection of MSCs in 4-week-old female NOD mice, or following the co-injection of MSCs and diabetogenic T cells in irradiated male NOD recipients, respectively. The frequency of CD4(+)FOXP3(+) cells and Foxp3 mRNA levels in the spleen of male NOD recipients were also quantified. In vivo cell homing was assessed by monitoring 5,6-carboxyfluorescein diacetate succinimidyl ester (CFSE)-labelled T cells or MSCs. In vitro, cell proliferation and cytokine production were assessed by adding graded doses of irradiated MSCs to insulin B9-23 peptide-specific T cell lines in the presence of irradiated splenocytes pulsed with the peptide. MSCs reduced the capacity of diabetogenic T cells to infiltrate pancreatic islets and to transfer diabetes. This protective effect was not associated with the modification of diabetogenic T cell homing, but correlated with a preferential migration of MSCs to pancreatic lymph nodes. While injection of diabetogenic T cells resulted in a decrease in levels of FOXP3(+) regulatory T cells, this decrease was inhibited by MSC co-transfer. Moreover, MSCs were able to suppress both allogeneic and insulin-specific proliferative responses in vitro. This suppressive effect was associated with the induction of IL10-secreting FOXP3(+) T cells [165].

Sixty NOD mice were divided into four groups, including normal control group, WJ-MSCs prevention group (before onset), WJ-MSCs treatment group (after onset), and diabetic control group. After homologous therapy, onset time of diabetes, levels of fasting plasma glucose (FPG), fed blood glucose and C-peptide, regulation of cytokines, and islet cells were examined and evaluated. After WJ-MSCs infusion, FPG and fed blood glucose in WJ-MSCs treatment group decreased to normal level in 6-8 days and maintained for 6 weeks. Level of fasting C-peptide of these mice was higher compared to diabetic control mice (P=0.027). In WJ-MSCs prevention group, WJ-MSCs played a protective role for 8-week delayed onset of diabetes, and fasting C-peptide in this group was higher compared to the other two diabetic groups (P=0.013, 0.035). Compared with diabetic control group, frequencies of CD4+CD25+Foxp3+ Tregs in WJ-MSCs prevention group and treatment group were higher, while levels of IL-2, IFN-γ, and TNF-α were lower (P<0.001); the degree of insulitis was also depressed, especially for WJ-MSCs prevention group (P<0.05). Infusion of WJ-MSCs could aid in T1DM through regulation of the autoimmunity and recovery of islet β-cells no matter before or after onset of T1DM. WJ-MSCs might be an effective method for T1DM [166].

MVs were purified from heterologous human MSCs by differential centrifugation.

Peripheral blood mononuclear cells (PBMCs) were obtained from patients with type 1 diabetes at disease onset, and responses to GAD65 stimulation were assessed by IFN-γ enzyme-linked immunosorbent spot analysis. Levels of cytokines and prostaglandin E2 (PGE2) were measured in the supernatant fraction, and T helper 17 (Th17) and regulatory T cell analysis was performed. MVs were internalised by PBMCs, as assessed by confocal microscopy and flow cytometry analyses. MVs significantly decreased IFN-γ spots and levels in GAD65-stimulated PBMCs, and significantly increased transforming growth factor-β (TGF-β), IL-10, IL-6 and PGE2 levels. Furthermore, MVs decreased the number of Th17 cells and the levels of IL-17, and increased FoxP3(+) regulatory T cells in GAD65-stimulated PBMCs. These results provide evidence that MSC-derived MVs can inhibit in vitro a proinflammatory response to an islet antigenic stimulus in type 1 diabetes. The action of MVs involves PGE2 and TGF-β signalling pathways and IL-10 secretion, suggesting a switch to an anti-inflammatory response of T cells [167].

In one study bone marrow derived MSCs were characterised and EVs were obtained by ultracentrifugation. DCs were differentiated from CD14(+) cells, obtained from nine type 1 diabetic patients at disease onset, pulsed with antigen GAD65 and cultured with MSCs or EVs. Levels of DC maturation and activation markers were evaluated by flow cytometry. GAD65-pulsed DCs and autologous CD14(−) cell were co-cultured and IFN-γ enzyme-linked immunosorbent spot responses were assayed. Secreted cytokine levels were measured and Th17 and regulatory T cells were analysed. MSC- and EV-conditioned DCs acquired an immature phenotype with reduced levels of activation markers and increased IL-10 and IL-6 production. Conditioned DC plus T cell co-cultures showed significantly decreased IFN-γ spots and secretion levels. Moreover, higher levels of TGF-β, IL-10 and IL-6 were detected compared with unconditioned DC plus T cell co-cultures. Conditioned DCs decreased Th17 cell numbers and IL-17 levels, and increased FOXP3(+) regulatory T cell numbers. EVs were internalised by DCs and EV-conditioned DCs exhibited a similar effect. In type 1 diabetes, MSCs induce immature IL-10-secreting DCs in vitro, thus potentially intercepting the priming and amplification of autoreactive T cells in tissue inflammation. These DCs can contribute to the inhibition of inflammatory T cell responses to islet antigens and the promotion of the anti-inflammatory, regulatory responses exerted by MSCs [168]

Pancreatitis

Mild AP was induced in Sprague-Dawley rats by 3 intraperitoneal injections of cerulein (100 μg/kg), given at 2-hour intervals; severe AP was induced by intraparenchymal injection of 3% sodium taurocholate solution. hcMSCs were labeled with CM-1,1′-dioctadecyl-3,3,3′-tetramethylindo-carbocyanine perchloride and administered to rats through the tail vein. hcMSCs underwent self-renewal and had multipotent differentiation capacities and immunoregulatory functions. Greater numbers of infused hcMSCs were detected in pancreas of rats with mild and severe AP than of control rats. Infused hcMSCs reduced acinar-cell degeneration, pancreatic edema, and inflammatory cell infiltration in each model of pancreatitis. The hcMSCs reduced expression of inflammation mediators and cytokines in rats with mild and severe AP. hcMSCs suppressed the mixed lymphocyte reaction and increased expression of Foxp3(+) (a marker of regulatory T cells) in cultured rat lymph node cells. Rats with mild or severe AP that were given infusions of hcMSCs had reduced numbers of CD3(+) T cells and increased expression of Foxp3(+) in pancreas tissues [169].

Multiple Sclerosis

In this pilot study a group of MS patients underwent MSC therapy and we assayed the expression of an X-linked transcription factor, FoxP3, as a specific marker of T Regulatory cells in peripheral blood, prior to and after the treatment. Using q RT-PCR for measurement of expression of FoxP3 by peripheral blood mononuclear cells, we found that in all subjects, except for one, the expression of FoxP3 at 6 months after intrathecal injection of MSC was significantly higher than the levels prior to treatment. Such significant enhanced expression of FoxP3 associated with clinical stability [170].

In another experiments, experimental autoimmune encephalomyelitis animal model was induced by injection of the MOG peptide, and mesenchymal stem cell injection was done 20 and 22 days after experimental autoimmune encephalomyelitis induction. Clinical scores were recorded daily to evaluate developing experimental autoimmune encephalomyelitis. The frequency of CD4(+)CD25(+)Foxp3(+) T cells in the spleen, the thymus, and the lymph nodes were analyzed by flow cytometry, and Foxp3, TGF-β1, and IL-10 mRNA were detected by reverse-transcription-polymerase chain reaction. Transplant of mesenchymal stem cells on experimental autoimmune encephalomyelitis mice led to a decreased clinical score, an up-regulation of CD4(+)CD25(+)Foxp3(+) T cells, Foxp3, TGF-β1, and IL-10 mRNA in the spleen, the lymph nodes, and the thymus as compared with experimental autoimmune encephalomyelitis mice. Transplant of mesenchymal stem cells may prevent developing experimental autoimmune encephalomyelitis and might be an available method in therapy of multiple sclerosis. Mesenchymal stem cells transplant may affect proliferation and function of CD4(+) T cells in experimental autoimmune encephalomyelitis, and CD4(+)CD25(+)Foxp3(+) T cell, Foxp3, TGF-β1, and IL-10 may be involved in this process [171].

Another study compared the immune regulatory properties of adipose tissue MSCs (AT-MSCs) in two independent routes of injection; namely intraperitoneal (i.p.) and intravenous (i.v.). We investigated the splenic CD4+CD25+FOXP3+ T cell population known as regulatory T cells, by flow cytometry and their brain cell infiltration by hematoxylin-eosin staining in both i.p. and i.v. routes of AT-MSC administration. We also evaluated the inflammatory cytokine profile including IFN-γ and IL-17 and anti-inflammatory cytokines such as IL-4 by ELISA technique in both routes of cell administration. We show that the i.p. route has a more pronounced effect in maintaining the splenic CD4+CD25+FOXP3+ T cell population and increase of IL-4 secretion. We also showed that i.p. injection of cells resulted in lower IFN-γ secretion and reduced cell infiltration in brain more effectively as compared to the i.v. route. The effects of AT-MSCs on down-regulation of splenocyte proliferation, IL-17 secretion and alleviating the severity of clinical scores were similar in i.p. and i.v. routes. Our data show that, due to their immunomodulative and neuroprotective effects, AT-MSCs may be a proper candidate for stem cell based MS therapy [172].

EAE was induced by immunization with myelin oligodendroglial glycoprotein peptide (MOG)35-55 in C57BL/6 mice. After immunization, mice were observed every 48 hours for signs of EAE and weight loss. At the onset of disease, approximately 14 days after immunization, EAE mice were subjected to a single intravenous injection of hPDLSCs (10(6) cells/150 μl) into the tail vein. At the point of animal sacrifice on day 56 after EAE induction, spinal cord and brain tissues were collected in order to perform histological evaluation, immunohistochemistry and western blotting analysis. Achieved results reveal that treatment with hPDLSCs may exert neuroprotective effects against EAE, diminishing both clinical signs and histological score typical of the disease (lymphocytic infiltration and demyelination) probably through the production of neurotrophic factors (results focused on brain-derived neurotrophic factor and nerve growth factor expression). Furthermore, administration of hPDLSCs modulates expression of inflammatory key markers (tumor necrosis factor-α, interleukin (IL)-1β, IL-10, glial fibrillary acidic protein, Nrf2 and Foxp3), the release of CD4 and CD8a T cells, and the triggering of apoptotic death pathway (data shown for cleaved caspase 3, p53 and p21). In light of the achieved results, transplantation of hPDLSCs may represent a putative novel and helpful tool for multiple sclerosis treatment. These cells could have considerable implication for future therapies for multiple sclerosis and this study may represent the starting point for further investigations [173].

A study conducted a comparative analysis of the immunomodulatory properties of adipose tissue mesenchymal stem cells (AT-MSCs) and their conditioned media (CM), derived from C57/BL6 mice, for mitigating the adverse clinical course of experimental autoimmune encephalomyelitis (EAE). We measure IL4, IL17 and IFNγ production of supernatant from spleen cells. We analyzed brain cell infiltration, splenocyte proliferation and evaluated the percentage of CD4+CD25+FOXP3+splenic cell population in all EAE C57/BL6 mice. AT-MSCs and its conditioned medium induced CD4+CD25+FOXP3+ regulatory T cells after in vitro co-culture with naïve T cells. There is no significant difference in the clinical scores and body weight of EAE mice treated with AT-MSCs and CM. The reduction in proliferative responses and brain cell infiltration was more pronounced in mice injected with CM than other groups. It is found that the percentage of splenic CD4+CD25+FOXP3+ population as well as the level of IL4 production in mice administrated with AT-MSCs is increased compared to other animals. Our results suggest that AT-MSCs-derived CM is promising in stem cell therapy, due to their neuroprotective and immunomudulatory properties [174].

he aim of this study was, therefore, to investigate the role of the IL17/IL17R pathway on MSCs immunoregulatory effects focusing on Th17 cell generation in vitro and on Th17-mediated EAE pathogenesis in vivo. In vitro, we showed that the immunosuppressive effect of MSCs on Th17 cell proliferation and differentiation is partially dependent on IL17RA expression. This was associated with a reduced expression level of MSCs immunosuppressive mediators such as VCAM1, ICAM1, and PD-L1 in IL17RA−/− MSCs as compared to wild-type (WT) MSCs. In the EAE model, we demonstrated that while WT MSCs significantly reduced the clinical scores of the disease, IL17RA−/− MSCs injected mice exhibited a clinical worsening of the disease. The disability of IL17RA−/− MSCs to reduce the progression of the disease paralleled the inability of these cells to reduce the frequency of Th17 cells in the draining lymph node of the mice as compared to WT MSCs. Moreover, we showed that the therapeutic effect of MSCs was correlated with the generation of classical Treg bearing the CD4+CD25+Foxp3+ signature in an IL17RA-dependent manner. Our findings reveal a novel role of IL17RA on MSCs immunosuppressive and therapeutic potential in EAE and suggest that the modulation of IL17RA in MSCs could represent a novel method to enhance their therapeutic effect in MS [175].

In one study investigators evaluated whether the combination of methylprednisolone (MP) and human bone marrow-derived mesenchymal stem cells (BM-MSCs) could enhance the therapeutic effectiveness in experimental autoimmune encephalomyelitis (EAE), a model for MS. EAE was induced by immunizing C57BL/6 mice with myelin oligodendrocyte glycoprotein 35-55 (MOG 35-55). The immunized mice received an intraperitoneal injection of MP (20 mg/kg), an intravenous injection of BM-MSCs (1×106 cells) or both on day 14 after immunization. Combination treatment significantly ameliorated the clinical symptoms, along with attenuating inflammatory infiltration and demyelination, compared to either treatment alone. Secretion of pro-inflammatory cytokines (IFN-γ, TNF-α, IL-17) was significantly reduced, and anti-inflammatory cytokines (IL-4, IL-10) was significantly increased by the combination treatment as compared to either treatment alone. Flow cytometry analysis of MOG-reactivated T cells in spleen showed that combination treatment reduced the number of CD4+CD45+ and CD8+ T cells, and increased the number of CD4+CD25+Foxp3+ regulatory T cells [176].

Investigators assessed and compared the therapeutic effects of human adipose-derived mesenchymal stem cells (hADSC) and hADSC-EVs from adipose tissue on experimental autoimmune encephalomyelitis (EAE). After induction of EAE in C57Bl/6 mice, they were treated with hADSCs, hADSC-EVs, or vehicle intravenously. The clinical score of all mice was recorded every other day. Mice were killed at Day 30 and splenocytes were isolated for proliferation assay and determination of the frequency of Treg cells by flow cytometry. Leukocyte infiltration by hematoxylin and eosin, percentages of demyelination areas by luxol fast blue, and mean fluorescence intensity of oligodendrocyte transcription factor 2 (OLIG2) and myelin basic protein (MBP) by immunohistochemistry were assessed in the spinal cord. Our results showed that the maximum mean clinical score and myelin oligodendrocyte glycoprotein-induced proliferation of splenocytes in hADSC- and hADSC-EV-treated mice were significantly lower than the control mice (p<0.05). We also demonstrated that the frequency of CD4+CD25+Foxp3+ cells was significantly higher in the spleen of hADSC-treated mice than EAE control mice (p=0.023). The inflammation score and the percentages of demyelination areas in hADSC- and hADSC-EV-treated groups significantly declined compared with the untreated control group (p<0.05). We also showed that there was no significant difference in MFI of MBP and OLIG2 in the spinal cord of studied groups. Overall, we suggest that intravenous administration of hADSC-EVs attenuates the induced EAE through diminishing proliferative potency of T cells, mean clinical score, leukocyte infiltration, and demyelination in a chronic model of multiple sclerosis [177].

Primary Biliary Cirrhosis

Primary biliary cirrhosis (PBC) is a slowly progressive autoimmune disease of unknown mechanism. We established a PBC animal model by injecting C57BL/6 mice with polyinosinic-polycytidylic acid sodium (polyL:C) to investigate the therapeutic effect of bone marrow-derived mesenchymal stem cells (BM-MSC) on this model. After 6 weeks of MSC infusion, serum aminotransferase and autoimmune antibodies declined, and histological examination by hematoxylin and eosin staining showed significant amelioration of monocytes infiltration around bile ducts of mice treated with BM-MSC. Interestingly, allogeneic BM-MSC transplantation markedly increased CD4(+)Foxp3(+) regulatory T cells in peripheral blood as well as in lymph nodes when analyzed by flow cytometry. Further examination showed serum TGF-β1 increased but IFN-γ decreased significantly in PBC mice treated with MSC, while with no obvious change in IL-10 expression. Our results for the first time suggested that BM-MSC transplantation could regulate systemic immune response and enhance recovery in liver inflammation of PBC mice, raising the possibility for clinical application of allogeneic MSC in treatment of early-stage PBC patients [178].

Ten patients were enrolled in this trial of BM-MSCT. All patients were permitted to concurrently continue their previous UDCA treatment. The efficacy of BM-MSCT in UDCA-resistant PBC was assessed at various time points throughout the 12-month follow up. No transplantation-related side effects were observed. The life quality of the patients was improved after BM-MSCT as demonstrated by responses to the PBC-40 questionnaire. Serum levels of ALT, AST, γ-GT, and IgM significantly decreased from baseline after BM-MSCT. In addition, the percentage of CD8+ T cells was reduced, while that of CD4+CD25+Foxp3+ T cells was increased in peripheral lymphocytic subsets. Serum levels of IL-10 were also elevated. Notably, the optimal therapeutic outcome was acquired in 3 to 6 months and could be maintained for 12 months after BM-MSCT. In conclusion, allogeneic BM-MSCT in UDCA-resistant PBC is safe and appears to be effective [179].

Liver Failure

In this prospective study, 56 patients were enrolled and randomly assigned to transplantation group and control group. After 24-week follow-up, 39 patients completed the study (20 cases in transplantation group and 19 cases in control group). The Model for End-Stage Liver Disease scores, liver function, changes of Treg/Th17 cells, as well as related transcription factors and serum cytokines, were determined. Although patients in both groups showed significant improvement after Entecavir treatment, ABMSC transplantation further improved patients' liver function. Moreover, there was a significant increase in Treg cells and a marked decrease in Th17 cells in the transplantation group compared with control, leading to an increased Treg/Th17 ratio. Furthermore, mRNA levels of Treg-related transcription factor (Foxp3) and Th17-related transcription factor (RORγt) were increased and decreased, respectively. In addition, serum transforming growth factor-β levels were significantly higher at early weeks of transplantation, while serum levels of interleukin-17, tumor necrosis factor-α, and interleukin-6 were significantly lower in patients in the transplantation group compared with control. [180].

An ALI model in C57BL/6 mice was induced by administration of intraperitoneal injection of CCl4. Transplanted ERCs were intravenously injected (1 million/mouse) into mice 30 min after ALI induction. Liver function, pathological and immunohistological changes, cell tracking, immune cell populations and cytokine profiles were assessed 24 h after the CCl4 induction. ERC treatment effectively decreased the CCl4-induced elevation of serum alanine aminotransferase (ALT) and aspartate aminotransferase (AST) activities and improved hepatic histopathological abnormalities compared to the untreated ALI group. Immunohistochemical staining showed that over-expression of lymphocyte antigen 6 complex, locus G (Ly6G) was markedly inhibited, whereas expression of proliferating cell nuclear antigen (PCNA) was increased after ERC treatment. Furthermore, the frequency of CD4+ and CD8+ T cell populations in the spleen was significantly down-regulated, while the percentage of splenic CD4+CD25+FOXP3+ regulatory T cells (Tregs) was obviously up-regulated after ERC treatment. Moreover, splenic dendritic cells in ERC-treated mice exhibited dramatically decreased MHC-II expression. Cell tracking studies showed that transplanted PKH26-labeled ERCs engrafted to lung, spleen and injured liver. Compared to untreated controls, mice treated with ERCs had lower levels of IL-1β, IL-6, and TNF-α but higher level of IL-10 in both serum and liver [181].

investigated molecular and cellular mechanisms involved in MSC-mediated modulation of IL17 signaling during acute liver injury. Single intravenous injection of MSCs attenuate acute hepatitis and hepatotoxicity of NKT cells in a paracrine, indoleamine 2,3-dioxygenase (IDO)-dependent manner. Decreased levels of inflammatory IL17 and increased levels of immunosuppressive IL10 in serum, reduced number of interleukin 17-producing natural killer T (NKT17) cells, and increased presence of forkhead box P3+IL10-producing natural killer T regulatory cells (NKTregs) were noticed in the injured livers of MSC-treated mice. MSCs did not significantly alter the total number of IL17-producing neutrophils, CD4+, and CD8+ T lymphocytes in the injured livers. Injection of mesenchymal stem cell-conditioned medium (MSC-CM) resulted with an increased NKTreg/NKT17 ratio in the liver and attenuated hepatitis in vivo and significantly reduced hepatotoxicity of NKT cells in vitro. This phenomenon was completely abrogated in the presence of IDO inhibitor, 1-methyltryptophan. In conclusion, the capacity of MSCs to alter NKT17/NKTreg ratio and suppress hepatotoxicity of NKT cells in an IDO-dependent manner may be used as a new therapeutic approach in IL17-driven liver inflammation [182].

Mice received CCl4 (1 μl/g intraperitoneally) twice/week for 1 month. MSCs (1×106), or MSC-conditioned medium (MSC-CM), were intravenously injected 24 h after CCl4 and on every 7th day. Liver fibrosis was determined by macroscopic examination, histological analysis, Sirius red staining, and RT-PCR. Serum levels of cytokines, indoleamine 2,3-dioxygenase (IDO), and kynurenine were determined by ELISA. Flow cytometry was performed to identify liver-infiltrated cells. In vitro, CD4+ T cells were stimulated and cultured with MSCs. 1-methyltryptophan was used for inhibition of IDO. MSCs significantly attenuated CCl4-induced liver fibrosis by decreasing serum levels of inflammatory IL-17, increasing immunosuppressive IL-10, IDO, and kynurenine, reducing number of IL-17 producing Th17 cells, and increasing percentage of CD4+IL-10+ T cells. Injection of MSC-CM resulted with attenuated fibrosis accompanied with the reduced number of Th17 cells in the liver and decreased serum levels of IL-17. MSC-CM promoted expansion of CD4+FoxP3+IL-10+ T regulatory cells and suppressed proliferation of Th17 cells. This phenomenon was completely abrogated in the presence of IDO inhibitor. MSCs, in IDO-dependent manner, suppress liver Th17 cells which lead to the attenuation of liver fibrosis [183].

Colitis

Mice with trinitrobenzene sulfonic acid-induced colitis were treated with hASCs after onset of disease and clinical scores were evaluated Inflammatory response was determined by measuring the levels of different inflammatory mediators in colon and serum. Th1-mediated effector responses were evaluated by determining the proliferation and cytokine profile of activated mesenteric lymph node cells. The number of regulatory T cells and the suppressive capacity on Th1 cell responses was determined Systemic infusion of hASCs or murine ASCs ameliorated the clinical and histopathologic severity of colitis, abrogating body weight loss, diarrhea, and inflammation and increasing survival (P<0.001). This therapeutic effect was mediated by down-regulating both Th1-driven autoimmune and inflammatory responses. ASCs decreased a wide panel of inflammatory cytokines and chemokines and increased interleukin-10 levels (P<0.001), directly acting on activated macrophages. hASCs also impaired Th1 cell expansion and induced/activated CD4(+)CD25(+)FoxP3(+) regulatory T cells with suppressive capacity on Th1 effector responses in vitro and in vivo (P<0.001) [184].

In another series of experiments, a rectal enema of trinitrobenzene sulfonic acid (TNBS) (100 mg/kg body weight) was administered to female BALB/c mice. Bone marrow mesenchymal stem cells (BMSCs) were derived from male green fluorescent protein (GFP) transgenic mice and were transplanted intravenously into the experimental animals after disease onset. Clinical activity scores and histological changes were evaluated. GFP and Sex determining region Y gene (SRY) expression were used for cell tracking. Ki67 positive cells and Lgr5-expressing cells were determined to measure proliferative activity. Inflammatory response was determined by measuring the levels of different inflammatory mediators in the colon and serum. The inflammatory cytokines included tumor necrosis factor-α (TNF-α), interferon-γ (IFN-γ), interleukin-2 (IL-2), IL-6, IL-17, IL-4, IL-10, and transforming growth factor (TGF-β). Master regulators of Th1 cells (T-box expressed in T cells, T-bet), Th17 cells (retinoid related orphan receptor gamma(t), RORγt), Th2 cells (GATA family of transcription factors 3, GATA3) and regulatory T cells (forkhead box P3, Foxp3) were also determined. Systemic infusion of GFP-BMSCs ameliorated the clinical and histopathologic severity of colitis, including body weight loss, diarrhea and inflammation, and increased survival (P<0.05). The cell tracking study showed that MSCs homed to the injured colon. MSCs promoted proliferation of intestinal epithelial cells and differentiation of intestinal stem cells (P<0.01). This therapeutic effect was mainly mediated by down-regulation of both Th1-Th17-driven autoimmune and inflammatory responses (IL-2, TNF-α, IFN-γ, T-bet; IL-6, IL-17, RORγt), and by up-regulation of Th2 activities (IL-4, IL-10, GATA-3) (P<0.05). MSCs also induced activated CD4(+)CD25(+)Foxp3(+) regulatory T cells (TGF-β, IL-10, Foxp3) with a suppressive capacity on Th1-Th17 effecter responses and promoted Th2 differentiation in vivo (P<0.05) [185].

Colitis was induced in mice by administration of dextran sulfate sodium or trinitrobenzene sulfonic acid. Mice then were given intraperitoneal injections of NOD2-activated hUCB-MSCs; colon tissues and mesenteric lymph nodes were collected for histologic analyses. A bromodeoxyuridine assay was used to determine the ability of hUCB-MSCs to inhibit proliferation of human mononuclear cells in culture. Administration of hUCB-MSCs reduced the severity of colitis in mice. The anti-inflammatory effects of hUCB-MSCs were greatly increased by activation of NOD2 by its ligand, muramyl dipeptide (MDP). Administration of NOD2-activated hUCB-MSCs increased anti-inflammatory responses in colons of mice, such as production of interleukin (IL)-10 and infiltration by T regulatory cells, and reduced production of inflammatory cytokines. Proliferation of mononuclear cells was inhibited significantly by co-culture with hUCB-MSCs that had been stimulated with MDP. MDP induced prolonged production of prostaglandin (PG)E2 in hUCB-MSCs via the NOD2-RIP2 pathway, which suppressed proliferation of mononuclear cells derived from hUCB. PGE2 produced by hUCB-MSCs in response to MDP increased production of IL-10 and T regulatory cells. In mice, production of PGE2 by MSCs and subsequent production of IL-10 were required to reduce the severity of colitis [186].

aimed to investigate whether MSCs can be used for the treatment of IBD through the induction of Tregs. MSCs were isolated and identified by flow cytometry. The MSCs were transduced with a replication-defective recombinant lentiviral vector carrying GFP in order to be able to trace the injected cells in vivo. Prepared MSCs (1×106) were injected into rats with 2,4,6-trinitrobenzene sulfonic acid (TNBS)-induced colitis via the tail vein; the control rats received phosphate-buffered saline (PBS) alone. Two weeks after the intravenous infusion, the frequency of CD4+CD25+Foxp3 cells in the peripheral blood was examined by flow cytometry. The colon was sectioned and analyzed for histopathological changes. Foxp3 mRNA expression was determined by real-time reverse-transcription polymerase chain reaction (qRT-PCR). In our study, the systemic infusion of MSCs significantly ameliorated the clinical and histopathologic severity of TNBS-induced colitis in contrast to the controls. There was an inverse regulation of mucosal and peripheral Foxp3 expression, suggesting that the MSCs redistributed the Tregs from the mucosa to the blood. Thus, MSCs exhibit immunomodulatory functions and may be used to ameliorate or treat IBD by redistributing regulatory T cells [187].

Colitis was induced by 4% dextran-sulfate-sodium (DSS, in drinking water) in BALB/c mice for 7 days. ERCs were cultured from healthy female menstrual blood, and injected (1 million/mouse/day, i.v.) into mice on days 2, 5, and 8 following colitis induction. Colonic and splenic tissues were collected on day 14 post-DSS-induction. Clinical signs, disease activity index (DAI), pathological and immunohistological changes, cytokine profiles and cell populations were evaluated. DSS-induced mice in untreated group developed severe colitis, characterized by body-weight loss, bloody stool, diarrhea, mucosal ulceration and colon shortening, as well as pathological changes of intra-colon cell infiltrations of neutrophils and Mac-1 positive cells. Notably, ERCs attenuated colitis with significantly reduced DAI, decreased levels of intra-colon IL-2 and TNF-α, but increased expressions of IL-4 and IL-10. Compared with those of untreated colitis mice, splenic dendritic cells isolated from ERC-treated mice exhibited significantly decreased MHC-II expression. ERC-treated mice also demonstrated much less CD3(+)CD25(+) active T cell and CD3(+)CD8(+) T cell population and significantly higher level of CD4(+)CD25(+)Foxp3(+) Treg cells. This study demonstrated novel anti-inflammatory and immunosuppressive effects of ERCs in attenuating colitis in mice, and suggested that the unique features of ERCs make them a promising therapeutic tool for the treatment of ulcerative colitis [188].

MSCs were isolated from bone marrow (BM-MSCs) of 4- to 6-week-old C57BL/6, C57BL/6-green fluorescent protein, or Balb/c Tsg6−/− male mice. Colitis was induced by ad libitum administration of dextran sulfate sodium for 10 days; after 5 days the mice were given intraperitoneal injections of BM-MSCs or saline (controls). Blood samples and intestinal tissues were collected 24, 48, 96, and 120 hours later; histologic and flow cytometry analyses were performed. Injection of BM-MSCs reduced colitis in mice, increasing body weight and reducing markers of intestinal inflammation, compared with control mice. However, fewer than 1% of MSCs reached the inflamed colon. Most of the BM-MSCs formed aggregates in the peritoneal cavity. The aggregates contained macrophages and B and T cells, and produced immune-regulatory molecules including FOXP3, interleukin (IL)10, transforming growth factor-β, arginase type II, chemokine (C-C motif) ligand 22 (CCL22), heme oxygenase-1, and TSG6. Serum from mice given BM-MSCs, compared with mice given saline, had increased levels of TSG6. Injection of TSG6 reduced the severity of colitis in mice, along with the numbers of CD45+ cells, neutrophils and metalloproteinase activity in the mucosa, while increasing the percentage of Foxp3CD45+ cells. TSG6 injection also promoted the expansion of regulatory macrophages that expressed IL10 and inducible nitric oxide synthase, and reduced serum levels of interferon-γ, IL6, and tumor necrosis factor. Tsg6−/− MSCs did not suppress the mucosal inflammatory response in mice with colitis. BM-MSCs injected into mice with colitis do not localize to the intestine but instead form aggregates in the peritoneum where they produce immunoregulatory molecules, including TSG6, that reduce intestinal inflammation. TSG6 is sufficient to reduce intestinal inflammation in mice with colitis [189].

In an interesting study, three different MSCs delivery routes: intraperitoneal (IP), intravenous (IV), and anal injection (AI) were compared on DSS-induced colitic mice model. The overall therapeutic factors, MSCs migration and targeting as well as local immunomodulatory cytokines and FoxP3(+) cells infiltration were analyzed. Colitis showed varying degrees of alleviation after three ways of MSCs transplantation, and the IP injection showed the highest survival rate of 87.5% and displayed the less weight loss and quick weight gain. The fecal occult blood test on the day 3 also showed nearly complete absence of occult blood in IP group. The fluorescence imaging disclosed higher intensity of engrafted cells in inflamed colon and the corresponding mesentery lymph nodes (MLNs) in IP and AI groups than the IV group. Real time-PCR and ELISA also demonstrate lower TNF-α and higher IL-10, TSG-6 levels in IP group. The immunohistochemistry indicated higher repair proliferation (Ki-67) and more FoxP3(+) cells accumulation of IP group. IP showed better colitis recovery and might be the optimum MSCs delivery route for the treatment of DSS-induced colitis [190].

Rats with DSS-induced colitis were divided into control and treatment groups: normal control group (rats fed with water), DSS group (rats fed with DSS solution), MSC group (DSS-treated rats injected intravenously with GFP-MSCs), IL-25-MSC group (DSS-treated rats injected intravenously with IL-25 primed GFP-MSCs), and mesalazine group (DSS-treated rats fed with mesalazine). In IL-25-MSC group, therapeutic efficacy (clinical symptoms) was better than in MSC group, but comparable to mesalazine group. In IL-25-MSC group and mesalazine group, fewer infiltrating inflammatory cells and lower pathological score were observed in the intestine. The FOXP3+ cells and IL-4+ cells decreased, but IL-17A+ cells and IFN-γ+ cells increased in the peripheral blood and colonic mucosa after DSS induced colitis, and these phenomena were reversed by MSC or mesalazine treatment. IL-17A+ cells reduced and FOXP3+ cells increased in IL-25-MSC group as compared with MSC group. The expressions of Ki67 and LGR5 were significantly elevated in MSC treatment groups as compared with normal control group, DSS group, and mesalazine group. Definite GFP positive cells were not observed in the intestine of MSC-treated rats. IL-25 primed MSCs exert improved therapeutic effects on the intestinal inflammation of IBD rats which may be related to the inhibition of Th17 immune response and induction of T Regulatory cell phenotype [191].

used a dextran sulfate sodium (DSS)-induced colitis mice model and treated them with IL-35-MSCs, MSCs or saline. The body weight was recorded daily and inflammatory processes were determined. Cytokine secretion by lamina propria lymphocytes (LPLs) and percentage of regulatory T cells (Tregs) were also measured: The data showed that mice in the two treated groups recovered their body weight more rapidly than mice treated with saline in the later stage of colitis. The colon lengths of IL-35-MSC-treated mice were markedly longer than those in the other two groups and the inflammation reduced significantly. Furthermore, the percentage of Foxp3+ Tregs increased significantly and the level of proinflammatory cytokines produced by LPLs decreased significantly in the IL-35-MSC-treated group. The results demonstrate that IL-35-MSCs could ameliorate ulcerative colitis by down-regulating the expression of pro-inflammatory cytokines [192].

The immune regulatory activities of MSC for generation of T regulatory cells can be seen in various species. For example, Intraperitoneal infusion of feline adipose tissue mesenchymal stem cells (fAT-MSC) ameliorated the clinical and histopathologic severity of colitis, including body weight loss, diarrhea, and inflammation in the colon of Dextran sulfate sodium (DSS)-treated mice (C57BL/6). Since regulatory T cells (Tregs) are pivotal in modulating immune responses and maintaining tolerance in colitis, the relation of Tregs with fAT-MSC-secreted factor was investigated in vitro. PGE2 secreted from fAT-MSC was demonstrated to induce elevation of FOXP3 mRNA expression and adjust inflammatory cytokines in Con A-induced feline peripheral blood mononuclear cells (PBMCs). Furthermore, in vivo, FOXP3+ cells of the fAT-MSC group were significantly increased in the inflamed colon, relative to that in the PBS group. These results suggest that PGE2 secreted from fAT-MSC can reduce inflammation by increasing FOXP3+ Tregs in mice model of colitis [193].

Crohn's

12 consecutive outpatients (eight males, median age 32 years) refractory to or unsuitable for current available therapies. MSCs were isolated from bone marrow and expanded ex vivo to be used for both therapeutic and experimental purposes. Ten patients (two refused) received intrafistular MSC injections (median 4) scheduled every 4 weeks, and were monitored by surgical, MRI and endoscopic evaluation for 12 months afterwards. The feasibility of obtaining at least 50×10⁶ MSCs from each patient, the appearance of adverse events, and the efficacy in terms of fistula healing and reduction of both Crohn's disease and perianal disease activity indexes were evaluated. In addition, the percentage of both mucosal and circulating regulatory T cells expressing FoxP3, and the ability of MSCs to influence mucosal T cell apoptosis were investigated. MSC expansion was successful in all cases; sustained complete closure (seven cases) or incomplete closure (three cases) of fistula tracks with a parallel reduction of Crohn's disease and perianal disease activity indexes (p<0.01 for both), and rectal mucosal healing were induced by treatment without any adverse effects. The percentage of mucosal and circulating regulatory T cells significantly increased during the treatment and remained stable until the end of follow up (p<0.0001 and p<0.01, respectively). Furthermore, MSCs have been proven to affect mucosal T cell apoptotic rate.

Arthritis

DBA/1 mice with collagen-induced arthritis were treated with human AD-MSCs after disease onset, and clinical scores were determined. Inflammatory response was determined by measuring the levels of different mediators of inflammation in the joints and serum. The Th1-mediated autoreactive response was evaluated by determining the proliferative response and cytokine profile of draining lymph node cells stimulated with the autoantigen. The number of Treg cells and the suppressive capacity on self-reactive Th1 cells were also determined. Systemic infusion of human AD-MSCs significantly reduced the incidence and severity of experimental arthritis. This therapeutic effect was mediated by down-regulating the 2 deleterious disease components: the Th1-driven autoimmune and inflammatory responses. Human AD-MSCs decreased the production of various inflammatory cytokines and chemokines, decreased antigen-specific Th1/Th17 cell expansion, and induced the production of antiinflammatory interleukin-10 in lymph nodes and joints. Human AD-MSCs also induced de novo generation of antigen-specific CD4+CD25+FoxP3+ Treg cells with the capacity to suppress self-reactive T effector responses [194].

DBA/1J mice with CIA were treated with syngeneic TGFβ-induced MSCs, whereas control mice received either vehicle or MSCs alone. Arthritis severity was assessed by clinical and histologic scoring. TGFβ-transduced MSCs were tested for their immunosuppressive ability and differential regulation in mice with CIA. T cell responses to type II collagen were evaluated by determining proliferative capacity and cytokine levels. The effects of TGFβ-transduced MSCs on osteoclast formation were analyzed in vitro and in vivo. Systemic infusion of syngeneic TGFβ-transduced MSCs prevented arthritis development and reduced bone erosion and cartilage destruction. Treatment with TGFβ-transduced MSCs potently suppressed type II collagen-specific T cell proliferation and down-regulated proinflammatory cytokine production. These therapeutic effects were associated with an increase in type II collagen-specific CD4+FoxP3+ Treg cells and inhibition of Th17 cell formation in the peritoneal cavity and spleen. Furthermore, TGFβ-transduced MSCs inhibited osteoclast differentiation [195].

CIA was induced in DBA/1J mice by immunization with type II collagen and Freund's complete adjuvant. G-MSCs were injected intravenously into the mice on day 14 after immunization. In some experiments, intraperitoneal injection of PC61 (anti-CD25 antibody) was used to deplete Treg cells in arthritic mice. Infusion of G-MSCs in DBA/1J mice with CIA significantly reduced the severity of arthritis, decreased the histopathology scores, and down-regulated the production of inflammatory cytokines (interferon-γ and interleukin-17A). Infusion of G-MSCs also resulted in increased levels of CD4+CD39+FoxP3+ cells in arthritic mice. These increases were noted early after infusion in the spleens and lymph nodes, and later after infusion in the synovial fluid. The FoxP3+ Treg cells that were increased in frequency mainly consisted of Helios-negative cells. When Treg cells were depleted, infusion of G-MSCs partially interfered with the progression of CIA. Pretreatment of G-MSCs with a CD39 or CD73 inhibitor significantly reversed the protective effect of G-MSCs on CIA. The role of G-MSCs in controlling the development and severity of CIA mostly depends on CD39/CD73 signals and partially depends on the induction of CD4+CD39+FoxP3+ Treg cells [196].

172 patients with active RA who had inadequate responses to traditional medication were enrolled. Patients were divided into two groups for different treatment: disease-modifying anti-rheumatic drugs (DMARDs) plus medium without UC-MSCs, or DMARDs plus UC-MSCs group (4×10(7) cells per time) via intravenous injection. Adverse events and the clinical information were recorded. Tests for serological markers to assess safety and disease activity were conducted. Serum levels of inflammatory chemokines/cytokines were measured, and lymphocyte subsets in peripheral blood were analyzed. No serious adverse effects were observed during or after infusion. The serum levels of tumor necrosis factor-alpha and interleukin-6 decreased after the first UC-MSCs treatment (P<0.05). The percentage of CD4(+)CD25(+)Foxp3(+) regulatory T cells of peripheral blood was increased (P<0.05). The treatment induced a significant remission of disease according to the American College of Rheumatology improvement criteria, the 28-joint disease activity score, and the Health Assessment Questionnaire. The therapeutic effects maintained for 3-6 months without continuous administration, correlating with the increased percentage of regulatory T cells of peripheral blood. Repeated infusion after this period can enhance the therapeutic efficacy. In comparison, there were no such benefits observed in control group of DMARDS plus medium without UC-MSCs. Thus, our data indicate that treatment with DMARDs plus UC-MSCs may provide safe, significant, and persistent clinical benefits for patients with active RA [197].

aimed to determine whether CTLA4Ig and human ASCs show synergistic effects on immunomodulation and clinical improvement of sustained severe rheumatoid arthritis in a mouse model. hASCs overexpressing CTLA4Ig (CTLA4Ig-hASC) were serially transplanted into mice with collagen-induced arthritis. Arthritic mice were subjected to four treatments based on their arthritis score on day 62 postimmunization: control (C group), hASC (H group), CTLA4Ig-hASC (CT group), and MTX (MTX group). A group of healthy mice was used as a normal control (N). Mice in the N and C groups were infused with 150 μl saline, and 2×10(6) hASCs or CTLA4Ig-hASCs in 150 μl of saline were intravenously administered to those in the H and CT groups, respectively, on days 63, 70, 77, and 84 after CII immunization. About 1 mg/kg of methotrexate was intraperitoneally administered to the MTX group three times a week for 4 weeks. Serial hASC and CTLA4Ig-hASC transplantation modulated various cytokines and chemokines related to the development of rheumatoid arthritis. Both treatments protected against destruction of cartilage, with CTLA4Ig-hASCs being most effective. Serum levels of CII autoantibodies and C-telopeptide of type II collagen were significantly low in the group transplanted with CTLA4Ig-hASCs. In vitro, ASC and CTLA4Ig-hASC treatment significantly decreased T-bet and GATA-3 expression in splenocytes from arthritic mice, and CTLA4Ig-hASC treatment significantly increased the ratio of Treg/Th17 (CD4(+)CD25(+)FoxP3(+)/CD4(+)CD25(+)RORγt) cells. Serial hASC and CTLA4Ig-hASC transplantation offers promising treatment for rheumatoid arthritis, and CTLA4Ig-hASCs showed stronger therapeutic effects than nontransduced hASCs [198].

reconstructed pcDNA3.1-IFNγ plasmids, transfected them to human umbilical cord derived MSCs, and detected the basic characters of MSCs including immune phenotype, cell vitality, proliferation, apoptosis and cell cycle progression after transfection. Subsequently, we analyzed the inhibition effect of IFN-γ-MSCs on T cell proliferation in vitro. Finally, we induced colitis in female C57BL/6 mice by 3% DSS treatment and evaluated the therapeutic efficacy of IFN-γ-MSCs on colitis. Transfection with pcDNA3.1-IFNγ did not change the basic characters of MSCs. Interestingly, IFN-γ-MSCs showed more potent immunosuppressive effects on the proliferation of T cells compared to normal MSCs. Furthermore, systemic infusion with IFN-γ-MSCs more efficiently ameliorated DSS-induced mouse colitis including colitis-related ease of body weight, increase of colon length, decrease of disease activity index, and improvement of small intestine tissues structure. In addition, IFN-γ-MSCs increased the populations of Foxp3(+) Tregs and Th2 cells both in mesenteric lymph node and spleen, upregulated indoleamine 2, 3-dioxygenase expression, and suppressed inflammatory cytokine production in mouse colon. Gene delivery with IFN-γ-expression plasmids enhanced the therapeutic effects of MSCs on DSS-induced mouse colitis. This study provides an effective therapeutic strategy of MSCs for inflammatory diseases [199].

a single dose of human expanded adipose-derived mesenchymal stem cells (eASCs) was administered to mice with established collagen-induced arthritis. A beneficial effect was observed soon after the infusion of the eASCs as shown by a significant decrease in the severity of arthritis. This was accompanied by reduced number of pathogenic GM-CSF(+) CD4(+) T cells in the spleen and peripheral blood and by an increase in the number of different subsets of regulatory T cells like FOXP3(+) CD4(+) T cells and IL10(+) IL17(−) CD4(+) T cells in the draining lymph nodes (LNs). Interestingly, increased numbers of Th17 cells coexpressing IL10 were also found in draining LNs. These results demonstrate that eASCs ameliorated arthritis after the onset of the disease by reducing the total number of pathogenic GM-CSF(+) CD4(+) T and by increasing the number of different subsets of regulatory T cells in draining LNs, including Th17 cells expressing IL10. All these cellular responses, ultimately, lead to the reestablishment of the regulatory/inflammatory balance in the draining LNs [200].

A combination of MSCs and Tr1 cells prevented the development of destructive arthritis compared to single cell therapy. These therapeutic effects were associated with an increase in type II collagen (CII)-specific CD4+CD25+Foxp3+ Treg cells and inhibition of CII-specific CD4+IL-17+ T cells. We observed that Tr1 cells produce high levels of IL-10-dependent interferon (IFN)-β, which induces toll-like receptor (TLR) 3 expression in MSCs. Moreover, induction of indoleamine 2,3-dioxygenase (IDO) by TLR3 involved an autocrine IFN-β that was dependent on STAT1 signaling. Furthermore, we observed that production of IFN-β and IL-10 in Tr1 cells synergistically induces IDO in MSCs through the STAT1 pathway. These findings suggest co-administration of MSCs and Tr1 cells to be a novel therapeutic modality for clinical autoimmune diseases [201].

Transplantation of MSC was assessed on established collagen-induced arthritis (CIA) were evaluated and compared to biologic therapies. CIA was induced with the immunisation of type II collagen (CII) in DBA/1 mice. Human umbilical cord MSC, anti-TNF antibody, rhTNFR:Fc fusion protein and anti-CD20 antibody were respectively injected intraperitoneally into CIA mice. Arthritis severity was assessed by clinical and histological scoring. The frequencies of lymphocytes in spleen were analysed, and serum concentrations of cytokines and autoantibody to CII were also measured. The ability of MSC to regulate the balance of T helper cell subsets in CII stimulated CIA CD4+ T cells was assessed in vitro. MSC treatment significantly decreased the severity of arthritis, which was comparable to biologic treatments. All the treatments down-regulated Th1 subset. Except anti-CD20 all the treatments decreased Th17 subset. MSC treatment enhanced the proportion of regulatory T (Treg) cells and inhibited the generation of T follicular helper (Tfh) cells. The decrease in autoantibody level was detectable in all the treated groups. In vitro MSC induced Foxp3+ T cells, and down-regulated IL-17+, IFNγ+ T cells and pathogenic IL-17+IFNγ+ or IL-17+Foxp3+ T cells. MSC also reduced the secretion of IL-1β, IL-6, IL-17 and TNF-α among collagen-specific T cells [202].

One study reported the feasibility and efficacy of the IL administration of human expanded adipose mesenchymal stem cells (eASCs) in a mouse model of collagen-induced arthritis (CIA). IL administration of eASCs attenuated the severity and progression of arthritis, reduced bone destruction and increased the levels of regulatory T cells (CD25+Foxp3+CD4+ cells) and Tr1 cells (IL10+CD4+), in spleen and draining lymph nodes. Taken together, these results indicate that IL administration of eASCs is very effective in modulating established CIA and may represent an alternative treatment modality for cell therapy with eASCs [203].

Male mice (age 7-9 weeks) were injected intra-articularly with SM-MSCs obtained from patients with osteoarthritis, on days 28, 32, and 38 after bovine type II collagen immunization. The efficacy of SM-MSCs in CIA was evaluated clinically and histologically. Cytokine profile analyses were performed by real-time polymerase chain reaction and multiplex analyses. Splenic helper T (Th) cell and regulatory B cell subsets were analyzed by flow cytometry. Intra-articular SM-MSC injection ameliorated the clinical and histological severity of arthritis. Decrease in tumor necrosis factor-α, interferon-γ, and interleukin- (IL-) 17A and increase in IL-10 production were observed after SM-MSC treatment. Flow cytometry showed that Th1 and Th17 cells decreased, whereas Th2, regulatory T (Treg), and PD-1+CXCR5+FoxP3+ follicular Treg cells increased in the spleens of SM-MSC-treated mice. Regulatory B cell analysis showed that CD21hiCD23hi transitional 2 cells, CD23lowCD21hi marginal zone cells, and CD19+CD5+CD1d+IL-10+ regulatory B cells increased following SM-MSC treatment [204].

evaluated the therapeutic potential of mesenchymal stem cell-conditioned medium (CM-MSC) as an alternative to cell therapy in an antigen-induced model of arthritis (AIA). Disease severity and cartilage loss were evaluated by histopathological analysis of arthritic knee joints and immunostaining of aggrecan neoepitopes. Cell proliferation was assessed for activated and naïve CD4+ T cells from healthy mice following culture with CM-MSC or co-culture with MSCs. T cell polarization was analysed in CD4+ T cells isolated from spleens and lymph nodes of arthritic mice treated with CM-MSC or MSCs. CM-MSC treatment significantly reduced knee-joint swelling, histopathological signs of AIA, cartilage loss and suppressed TNFα induction. Proliferation of CD4+ cells from spleens of healthy mice was not affected by CM-MSC but reduced when cells were co-cultured with MSCs. In the presence of CM-MSC or MSCs, increases in IL-10 concentration were observed in culture medium. Finally, CD4+ T cells from arthritic mice treated with CM-MSC showed increases in FOXP3 and IL-4 expression and positively affected the Treg:Th17 balance in the tissue. CM-MSC treatment reduces cartilage damage and suppresses immune responses by reducing aggrecan cleavage, enhancing Treg function and adjusting the Treg:Th17 ratio. CM-MSC may provide an effective cell-free therapy for inflammatory arthritis [205].

study was to evaluate the therapeutic effects of human MSCs on inflammatory arthritis and to identify the underlying mechanisms. Mice with collagen antibody-induced arthritis (CAIA) received two intraperitoneal injections of human bone marrow-derived MSCs. The clinical and histological features of injected CAIA were then compared with those of non-injected mice. The effect of MSCs on induction of regulatory T cells was examined both in vitro and in vivo. We also examined multiple cytokines secreted by peritoneal mononuclear cells, along with migration of MSCs in the presence of stromal cell-derived factor-1 alpha (SDF-1a) and/or regulated on activation, normal T cell expressed and secreted (RANTES). Sections of CAIA mouse joints and spleen were stained for human anti-nuclear antibodies (ANAs) to confirm migration of injected human MSCs. The results showed that MSCs alleviated the clinical and histological signs of synovitis in CAIA mice. Peritoneal lavage cells from mice treated with MSCs expressed higher levels of SDF-1a and RANTES than those from mice not treated with MSCs. MSC migration was more prevalent in the presence of SDF-1a and/or RANTES. MSCs induced CD4+ T cells to differentiate into regulatory T cells in vitro, and expression of FOXP3 mRNA was upregulated in the forepaws of MSC-treated CAIA mice. Synovial and splenic tissues from CAIA mice receiving human MSCs were positive for human ANA, suggesting recruitment of MSCs. Taken together, these results suggest that MSCs migrate into inflamed tissues and directly induce the differentiation of CD4+ T cells into regulatory T cells, which then suppress inflammation. Thus, systemic administration of MSCs may be a therapeutic option for rheumatoid arthritis [206].

Investigators monitored the profile of immune cells in fresh peripheral blood after IA injection of autologous ASCs in the knee of 18 patients with severe OA (ADIPOA phase I study). Specifically, we used 8-color flow cytometry antibody panels to characterize the frequencies of innate and adaptive immune cell subsets (monocytes, dendritic cells, regulatory T cells and B cells) in blood samples at baseline (before injection) and one week, one month and three months after ASC injection. Results: We found that the percentage of CD4+CD25highCD127lowFOXP3+ regulatory T cells was significantly increased at 1 month after ASC injection, and this effect persisted for at least 3 months. Moreover, CD24highCD38high transitional B cells also were increased, whereas the percentage of classical CD14+ monocytes was decreased, at 3 months after ASC injection. These results suggest a global switch toward regulatory immune cells following IA injection of ASCs, underscoring the safety of ASC-based therapy. We did not find any correlation between the scores for the Visual Analogic Scale for pain, the Western Ontario and McMaster Universities Osteoarthritis Index (pain subscale and total score) at baseline and the immune cell profile changes, but this could be due to the small number of analyzed patients [207].

a single dose of autologous MSCs isolated from bone marrow (autologous BM-MSCs, 1×106 per kg) was injected intravenously into 13 patients suffering from refractory RA who were followed up within 12 months after the intervention to evaluate immunological elements. Our results showed that the gene expression of forkhead box P3 (FOXP3) in peripheral blood mononuclear cells (PBMCs) considerably increased at month 12. We found a substantial increasing trend in the culture supernatant levels of IL-10 and transforming growth factor-beta 1 (TGF-β1) in PBMCs from the beginning of the intervention up to the end. Our data may reflect the sufficient immunoregulatory effect of autologous BM-MSCs on regulatory T cells in patients suffering from refractory RA [208].

Lupus

The safety and efficacy of bone marrow (BM)-derived MSCs in two SLE patients was evaluated; the suppressor effect of these cells in-vitro and the change in CD4+CD25+FoxP3+ T regulatory (Treg) cells in response to treatment. Two females (JQ and SA) of 19 and 25 years of age, fulfilling the 1997 American College of Rheumatology (ACR) criteria for SLE were infused with autologous BM-derived MSCs. Disease activity indexes and immunological parameters were assessed at baseline, 1, 2, 7 and 14 weeks. Peripheral blood lymphocyte (PBL) subsets and Treg cells were quantitated by flow cytometry, and MSCs tested for in-vitro suppression of activation and proliferation of normal PBLs. No adverse effects or change in disease activity indexes were noted during 14 weeks of follow-up, although circulating Treg cells increased markedly. Patient MSCs effectively suppressed in-vitro PBL function. However, JQ developed overt renal disease 4 months after infusion. MSC infusion was without adverse effects, but did not modify initial disease activity in spite of increasing CD4+CD25+FoxP3+ cell counts. One patient subsequently had a renal flare. We speculate that the suppressive effects of MSC-induced Treg cells might be dependent on a more inflammatory milieu, becoming clinically evident in patients with higher degrees of disease activity [209].

(NZBxNZW)F1 mice with SLE were administered human AD-MSCs (5×10(5)) intravenously every 2 weeks from age 6 weeks until age 60 weeks, while the control group received saline vehicle on the same schedule. Another experiment was carried out with a different initiation time point for serial transplantation (age 6 weeks or age 32 weeks). Long-term serial administration (total of 28 times) of human AD-MSCs ameliorated SLE without any adverse effects. Compared with the control group, the human AD-MSC-treated group had a significantly higher survival rate with improvement of histologic and serologic abnormalities and immunologic function, and also had a decreased incidence of proteinuria. Anti-double-stranded DNA antibodies and blood urea nitrogen levels decreased significantly with transplantation of human AD-MSCs, and serum levels of granulocyte-macrophage colony-stimulating factor, interleukin-4 (IL-4), and IL-10 increased significantly. A significant increase in the proportion of CD4+FoxP3+ cells and a marked restoration of capacity for cytokine production were observed in spleens from the human AD-MSC-treated group. In the second experiment, an early stage treatment group showed better results (higher survival rates and lower incidence of proteinuria) than an advanced stage treatment group [210].

a case of autologous HSC transplantation combined with MSCs in a 25-year-old severe SLE patient with multiple life-threatening complications and refractory to conventional cyclophosphamide (CYC) therapy. After being pretreated with CYC, fludarabine and antithymocyte globulin, the patient was transplanted with autologous CD34+HSCs and MSCs by intravenous infusion. Hematopoietic regeneration was observed on day 12 thereafter. After HSC and MSC transplantation, the patient's clinical symptoms caused by SLE were remitted, and the SLEDAI score decreased. Moreover, CD4+CD25+FoxP3+ Treg cells increased in peripheral blood mononuclear cells (PBMCs) after transplantation. This result suggests that the combined transplantation of HSCs and MSCs may reset the adaptive immune system to re-establish self-tolerance in SLE. A 36-month follow-up showed that the clinical symptoms remained in remission. Although a longer follow-up is required for assessing the long-term efficacy, our present results suggest that the combined transplantation of HSCs and MSCs may be a novel and effective therapy for refractory SLE [211].

Thirty patients with active SLE, refractory to conventional therapies, were given UC MSCs infusions. The percentages of peripheral blood CD4+CD25+Foxp3+ regulatory T cells (Treg) and CD3+CD8-IL17A+Th17 cells and the mean fluorescence intensities (MFI) of Foxp3 and IL-17 were measured at 1 week, 1 month, 3 months, 6 months, and 12 months after MSCs transplantation (MSCT). Serum cytokines, including transforming growth factor beta (TGF-β), tumor necrosis factor alpha (TNF-α), interleukin 6 (IL-6), and IL-17A were detected using ELISA. Peripheral blood mononuclear cells from patients were collected and co-cultured with UC MSCs at ratios of 1:1, 10:1, and 50:1, respectively, for 72 h to detect the proportions of Treg and Th17 cells and the MFIs of Foxp3 and IL-17 were determined by flow cytometry. The cytokines in the supernatant solution were detected using ELISA. Inhibitors targeting TGF-β, IL-6, indoleamine 2,3-dioxygenase (IDO), and prostaglandin E2 were added to the co-culture system, and the percentages of Treg and Th17 cells were observed. The percentage of peripheral Treg and Foxp3 MFI increased 1 week, 1 month, and 3 months after UC MSCs transplantation, while the Th17 proportion and MFI of IL-17 decreased 3 months, 6 months, and 12 months after the treatment, along with an increase in serum TGF-β at 1 week, 3 months, and 12 months and a decrease in serum TNF-α beginning at 1 week. There were no alterations in serums IL-6 and IL-17A before or after MSCT. In vitro studies showed that the UC MSCs dose-dependently up-regulated peripheral Treg proportion in SLE patients, which was not depended on cell-cell contact. However, the down-regulation of Th17 cells was not dose-dependently and also not depended on cell-cell contact. Supernatant TGF-β and IL-6 levels significantly increased, TNF-α significantly decreased, but IL-17A had no change after the co-culture. The addition of anti-TGF-β antibody significantly abrogated the up-regulation of Treg, and the addition of PGE2 inhibitor significantly abrogated the down-regulation of Th17 cells. Both anti-IL-6 antibody and IDO inhibitor had no effects on Treg and Th17 cells: UC MSCs up-regulate Treg and down-regulate Th17 cells through the regulation of TGF-β and PGE2 in lupus patients [212].

Adipose-derived stem cells (ADSCs), stromal cells derived from adipose tissue, were investigated with allogeneic ADSCs in B6.MRL/lpr mice, a murine model of systemic lupus erythematosus (SLE). We intravenously injected allogeneic ADSCs into SLE mice after disease onset and report that ADSCs reduced anti-ds DNA antibodies in serum and proteinuria in SLE mice. Also, ADSCs decreased IL-17 and IL-6 expression in serum of SLE mice. ADSCs alleviated renal damage and inflammatory cell infiltration and edema of the renal interstitium. Furthermore, ADSCs significantly downregulated renal IL-17 and CD68 expression, suggesting that ADSCs suppressed renal inflammation. ADSCs also decreased IL-17 mRNA expression and increased Foxp3, ROR-γt and miR-23b mRNA expression in renal tissue in SLE mice. ADSCs reduced renal protein expression of TAB 2 and IKK-α in SLE mice. Thus, ADSCs may be a novel potential therapy for treating SLE [213].

A study was conducted to explore whether sHLA-G is involved in upregulating effects of MSCs on Treg, which contributes to therapeutic effects of MSCs transplantation in SLE. The serum sHLA-G levels of SLE patients and healthy controls were detected by ELISA. The percentages of peripheral blood CD4+ILT2+, CD8+ILT2+, CD19+ILT2+ cells and Treg cells were examined by flow cytometry. Ten patients with active SLE, refractory to conventional therapies, were infused with umbilical cord derived MSCs (UC-MSCs) and serum sHLA-G was measured 24 h and 1 month after infusion. The mice were divided into three groups: C57BL/6 mice, B6.MRL-Faslpr mice infused with phosphate buffer saline (PBS), and B6.MRL-Faslpr mice infused with bone marrow MSCs (BM-MSCs). Then, the concentrations of serum Qa-2 were detected. Peripheral blood mononuclear cells (PBMCs) were isolated from SLE patients and co-cultured with UC-MSCs for 3 days at different ratios (50:1, 10:1, and 2:1) with or without HLA-G antibody, and the frequencies of CD4+CD25+Foxp3+ T cells were then determined by flow cytometry. The concentrations of serum sHLA-G were comparable between SLE patients and healthy controls. However, there was a negative correlation between sHLA-G levels and SLE disease activity index (SLEDAI) scores in active SLE patients (SLEDAI>4). We found that serum sHLA-G levels were negatively correlated with blood urea nitrogen, serum creatinine and 24-hour urine protein in SLE patients. The sHLA-G levels were significantly lower in SLE patients with renal involvement than those without renal involvement. The expression of ILT2 on CD4+ T cells from SLE patients decreased significantly compared to that of healthy controls. A positive correlation between the frequencies of Treg and CD4+ILT2+ T cells was found in SLE patients. The levels of sHLA-G increased 24 h post UC-MSCs transplantation. The concentrations of Qa-2 in BM-MSCs transplanted mice were significantly higher than those of control group. In vitro studies showed that MSCs increased the frequency of Treg cells in SLE patients in a dose-dependent manner, which was partly abrogated by the anti-HLA-G antibody. Ther results suggested that MSCs may alleviate SLE through upregulating Treg cells, which was partly dependent on sHLA-G [214].

Autoimmune Uveitis

The authors intravenously injected syngeneic (isolated from Lewis rats) or allogeneic (isolated from Wistar rats) MSCs into IRBP-induced EAU Lewis rats, either before disease onset (simultaneous with immunization, preventive protocol) or at different time points after disease onset (therapeutic protocol). T-cell response to IRBP 1169-1191 from MSC-treated rats was evaluated, Th1/Th2/Th17 cytokines produced by lymphocytes were measured, and CD4(+)CD25(+) regulatory T cells (Treg) were detected. MSC administration before disease onset not only strikingly reduced the severity of EAU, it also delayed the onset of the disease. MSC administration was also effective after disease onset and at the peak of disease, but not after disease stabilization. Clinical efficacy for all treatments was consistent with reduced cellular infiltrates and milder uveal and retinal impairment. T-cell response to IRBP 1169-1191 from MSC-treated rats was inhibited. MSCs significantly decreased the production of IFN-γ and IL-17 and increased the production of IL-10 of T lymphocytes from EAU rats either in vivo or in vitro [215].

here investigated whether intraperitoneal administration of human mesenchymal stem/stromal cells (hMSCs) might prevent development of experimental autoimmune uveitis (EAU) in mice. Time course study showed that the number of IFN-γ- or IL-17-expressing CD4(+) T cells was increased in draining lymph nodes (DLNs) on the postimmunization day 7 and decreased thereafter. The retinal structure was severely disrupted on day 21. An intraperitoneal injection of hMSCs at the time of immunization protected the retina from damage and suppressed the levels of proinflammatory cytokines in the eye. Analysis of DLNs on day 7 showed that hMSCs decreased the number of Th1 and Th17 cells. The hMSCs did not reduce the levels of IL-1β, IL-6, IL-12, and IL-23 which are the cytokines that drive Th1/Th17 differentiation. Also, hMSCs did not induce CD4(+)CD25(+)Foxp3(+) cells. However, hMSCs increased the level of an immunoregulatory cytokine IL-10 and the population of IL-10-expressing B220(+)CD19(+) cells. Together, data demonstrate that hMSCs attenuate EAU by suppressing Th1/Th17 cells and induce IL-10-expressing B220(+)CD19(+) cells [216].

Autoimmune Hearing Loss

examine the immunosuppressive activity of hASCs on autoreactive T cells from the experimental autoimmune hearing loss (EAHL) murine model. Female BALB/c mice underwent β-tubulin immunization to develop EAHL; mice with EAHL were given hASCs or PBS intraperitoneally once a week for 6 consecutive weeks. Auditory brainstem responses were examined over time. The T helper type 1 (Th1)/Th17-mediated autoreactive responses were examined by determining the proliferative response and cytokine profile of splenocytes stimulated with β-tubulin. The frequency of regulatory T (Treg) cells and their suppressive capacity on autoreactive T cells were also determined. Systemic infusion of hASCs significantly improved hearing function and protected hair cells in established EAHL. The hASCs decreased the proliferation of antigen-specific Th1/Th17 cells and induced the production of anti-inflammatory cytokine interleukin-10 in splenocytes. They also induced the generation of antigen-specific CD4(+) CD25(+) Foxp3(+) Treg cells with the capacity to suppress autoantigen-specific T-cell responses. The experiment demonstrated that hASCs are one of the important regulators of immune tolerance with the capacity to suppress effector T cells and to induce the generation of antigen-specific Treg cells [217].

study was to examine the activities of hASCs (Human Adipose tissue Derived Stem Cells) on experimental autoimmune hearing loss (EAHL) and how human stem cells regenerated mouse cochlea cells. We have restored hearing in 19 years old white female with autoimmune hearing loss with autologous adipose tissue derived stem cells and we wish to understand the mechanism of restoration of hearing in animal model. BALB/c mice underwent to develop EAHL; mice with EAHL were given hASCs intraperitoneally once a week for 6 consecutive weeks. ABR were examined over time. The helper type 1 autoreactive responses and T-reg cells were examined. H&E staining or immunostaining with APC conjugated anti-HLA-ABC antibody were conducted. The organ of Corti, stria vascularis, spira ligament and spiral ganglion in stem cell group are normal. In control group, without receiving stem cells, the organ of Corti is replaced by a single layer of cells, atrophy of stria vascularis. Systemic infusion of hASCs significantly improved hearing function and protected hair cells in established EAHL. The hASCs decreased the proliferation of antigen specific Th1/Th17 cells and induced the production of anti-inflammatory cytokine interleukin10 in splenocytes. They also induced the generation of antigen specific CD4(+)CD25(+)Foxp3(+) T-reg cells. The experiment showed the restoration is due to the paracrine activities of human stem cells, since there are newly regenerated mice spiral ganglion cells, not human mesenchymal stem cells derived tissue given by intraperitoneally [218].

Autoimmune Thyroiditis

A preclinical study by using large-sized lab animals and applying ASCs that overexpress therapeutic genes. Experimental autoimmune thyroiditis was induced by immunization with thyroglobulin. Experimental dogs were divided into five groups: (i) ASC IT+IV, (ii) ASC IV, (iii) CTLA4Ig-ASC IT+IV, (iv) CTLA4Ig-ASC IV, and (v) control IT+IV (saline only), and they received intrathyroidal (IT; 10 million cells/250 μl saline per thyroid) administration one time or intravenous (IV; 20 million cells/5 ml) administration seven times within a 101-day period. Blood samples were collected every week, and thyroids were harvested on days 104-106. After serial ASC or CTLA4Ig transplantation, the levels of canine thyroglobulin autoantibodies (TgAA) in serum and the infiltration of T-lymphocytes between the follicles of the thyroid glands were decreased. The expression of FoxP3 in submandibular lymph nodes was significantly increased. Repeated long-term administration of autologous ASCs or CTLA4Ig-ASCs did not generate changes in clinical chemistry parameters or humoral responses. The TgAA test can detect autoimmune thyroiditis years before clinical signs of hypothyroidism occur. Thus, ASC and CTLA4Ig-ASC transplantation in that period can be attractive candidates to ameliorate autoimmune thyroiditis and prevent the development of hypothyroidism [219].

Lung Inflammation

Mice were administered intratracheally endotoxin (lipopolysaccharide [LPS]) and received intrapulmonary 1×10(6) UCMSC 4 hours after challenge. The CD4(+)CD25(+) Foxp3(+) Treg, survival time, body weight, histology and lung injury scores were assessed after transplantation of UCMSC. In addition, anti-inflammatory factor IL10 and pro-inflammatory mediators production including tumor necrosis factor-α (TNF-α), macrophage inflammatory protein-2(MIP-2) and interferon-γ (IFN-γ) were detected. Transplantation of UCMSC resulted in significant increase in the level of CD4(+)CD25(+) Foxp3(+) Treg in ALI. Increased level of anti-inflammatory factor IL-10 and reduced levels of TNF-α, MIP-2 and IFN-γ were simultaneously observed in ALI in comparison with control mice. Our data demonstrate for the first time that transplantation of UCMSC ameliorates ALI by enhancing the diminished levels of alveolar CD4(+)CD25(+) Foxp3(+) Treg and balancing anti- and pro-inflammatory factors in ALI mice [220].

Male Sprague-Dawley rats (n=6/group) underwent LCHS with or without a single intravenous dose of 5×10(6) Sprague-Dawley rat MSCs after resuscitation. Thereafter, rats were subjected to 2 hours of CS daily on days 1-6 and were humanely killed on day 7. Lung histology was scored according to a well-established lung injury score (LIS) that included interstitial and pulmonary edema, alveolar integrity, and inflammatory cells. Scoring ranges from 0 (normal lung) to 11 (most severely injured). Whole blood was analyzed for the presence of CD4(+)CD25(+)FoxP3(+) T-regulatory cells (Treg) by flow cytometry: Seven days after isolated LC, LIS had returned to 0.8±0.4; however, after LCHS/CS healing is significantly delayed (7.2±2.2; P<0.05). Addition of MSC to LCHS/CS decreased LIS to 2.0±1.3 (P<0.05) and decreased all subgroup scores (inflammatory cells, interstitial and pulmonary edema, and alveolar integrity) significantly compared with LCHS/CS (P<0.05). The percentage of Tregs found in the peripheral blood of animals undergoing LCHS/CS did not change from LC alone (10.5±3.3% vs 6.7±1.7%; P>0.05). Treatment with MSCs significantly increased the Treg population compared with LCHS/CS alone (11.7±2.7% vs 6.7±1.7%; P<0.05) CONCLUSION: In this model, severe impairment of wound healing observed 1 week after LCHS/CS is reversed by a single treatment with MSCs immediately after resuscitation. This improvement in lung healing is associated with a decrease in the number of inflammatory cells and lung edema and a significant increase in peripheral Tregs. Further study into the timing of administration and mechanisms by which cell-based therapy using MSCs modulate the immune system and improve wound healing is warranted [221].

Kidney Failure

study was to determine the role of CD11c⁺ cells in MSC-induced effects on ischemia/reperfusion injury (IRI). IRI was induced in wildtype (WT) mice and CD11c⁺-depleted mice following pretreatment with or without MSCs. In the in-vitro experiments, the MSC-treated CD11c⁺ cells acquired regulatory phenotype with increased intracellular IL-10 production. Although splenocytes cocultured with MSCs showed reduced T cell proliferation and expansion of CD4⁺FoxP3⁺ regulatory T cells (Tregs), depletion of CD11c⁺ cells was associated with partial loss of MSCs effect on T cells. In in-vivo experiment, MSCs' renoprotective effect was also associated with induction of more immature CD11⁺ cells and increased FoxP3 expression in I/R kidneys. However all these effects induced by the MSCs were partially abrogated when CD11c⁺ cells were depleted in the CD11c⁻ DTR transgenic mice. In addition, the observation that adoptive transfer of WT CD11c⁺ cells partially restored the beneficial effect of the MSCs, while transferring IL-10 deficient CD11c⁺ cells did not, strongly suggest the important contribution of IL-10 producing CD11c⁺ cells in attenuating kidney injury by MSCs [222].

A model of AKI induced by intravenous administration of 5 mg/kg cisplatin. BM-MSCs were transplanted through intra-arterial injection. The animals were followed for survival, biochemistry analysis and pathology. Transplantation of 5×10(6) cells/kg ameliorated renal function during the first week, as shown by significantly lower serum creatinine and urea values and higher urine creatinine and urea clearance without hyponatremia, hyperkalemia, proteinuria and polyuria up to 84 d compared with the vehicle and control groups. The superparamagnetic iron oxide nanoparticle-labeled cells were found in both the glomeruli and tubules. BM-MSCs markedly accelerated Foxp3+ T-regulatory cells in response to cisplatin-induced damage, as revealed by higher numbers of Foxp3+ cells within the tubuli of these monkeys compared with cisplatin-treated monkeys in the control and vehicle groups. These data demonstrate that BM-MSCs in this unique large-animal model of cisplatin-induced AKI exhibited recovery and protective properties [223].

demonstrated that administration of IL-17A-pretreated MSCs resulted in significantly lower acute tubular necrosis scores, serum creatinine, and BUN of mice with IRI-AKI, compared with the administration of MSCs. Of the co-cultured splenocytes, IL-17A-pretreated MSCs significantly increased the percentages of CD4+Foxp3+ Tregs and decreased concanavalin A-induced T cell proliferation. Furthermore, mice with IRI-AKI that underwent IL-17A-pretreated MSC therapy had significantly lower serum IL-6, TNF-α, and IFN-γ levels, a higher serum IL-10 level, and higher spleen and kidney Treg percentages than the mice that underwent MSCs treatment. Additionally, the depletion of Tregs by PC61 (anti-CD25 antibody) reversed the enhanced treatment efficacy of the IL-17A-pretreated MSCs on mice with IRI-AKI. Additionally, IL-17A upregulated COX-2 expression and increased PGE2 production. The blockage of COX-2 by celecoxib reversed the benefit of IL-pretreated 17A-MSCs on the serum PGE2 concentration, spleen and kidney Tregs percentages, serum creatinine and BUN levels, renal acute tubular necrosis scores, and serum IL-6, TNF-α, IFN-γ, and IL-10 levels of IRI-pretreated mice with AKI, compared with MSCs. Thus, our results suggest that IL-17A pretreatment enhances the efficacy of MSCs on mice with IRI-AKI by increasing the Treg percentages through the COX-2/PGE2 pathway [224].

Premature Overian Failure

effects of human adipose-derived mesenchymal stem cells (hADSCs) combined with estrogen on regulatory T cells (Tregs) in patients with premature ovarian insufficiency (POI): hADSCs were isolated by enzymatic digestion and identified by flow cytometry. Peripheral blood mononuclear cells (PBMCs) were isolated from POI patients and healthy controls. PBMCs were cultured in the following experimental groups: the control group, hADSC group, estrogen group and combined group. The PBMCs in the hADSC group were co-cultured with hADSCs at concentrations of 1×104, 2×104, or 1×105 cells/well, and the estrogen group was co-cultured with 10-9, 10-8, or 10-7 mol/L 17β-estradiol. Cell proliferation was measured with the CCK-8 assay. The percentage of CD4+CD25+Foxp3+ Tregs was measured by flow cytometry. The expression levels of Foxp3, TGF-β1 and IFN-γ were measured by real-time PCR Treatment with hADSCs, estrogen and their combination promoted Tregs differentiation of PBMCs from POI patients and healthy controls. An increase in the percentage of CD4+CD25+Foxp3+ Tregs was observed when PBMCs were co-cultured with hADSCs, 17β-estradiol and their combination. Foxp3 and TGF-β1 mRNA expression was higher and IFN-γ mRNA expression was lower in the hADSCs, estrogen and combined groups than in the control group. Combined treatment with hADSCs and estrogen played an immunomodulatory role by promoting Tregs proliferation, thereby potentially improving impaired ovarian function [225].

For the practice of the invention, MSC are used to reprogramme immune cells in order to endow renal regenerative activity can be utilized as was previously performed in clinical trials with non-selected MSC. “Mesenchymal stem cell” or “MSC” in some embodiments refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage. Other cells possessing mesenchymal-like properties are included within the definition of “mesenchymal stem cell”, with the condition that said cells possess at least one of the following: a) regenerative activity; b) production of growth factors; c) ability to induce a healing response, either directly, or through elicitation of endogenous host repair mechanisms. As used herein, “mesenchymal stromal cell” or ore mesenchymal stem cell can be used interchangeably. Said MSC can be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may includes cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™, Stemedyne™-MSC, Stempeucel®, StempeucelCLl, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).

In some embodiments, dendritic cells are generated which possess tolerogenic activity and said dendritic cells are pulsed with renal antigens. Said renal antigens include the myosin heavy chain [1]. Said dendritic cells can be made tolerogenic by culture in cytokines such as IL-10 or they can be further made tolerogenic by culture with regenerative cells. In one embodiment said regenerative cells are mesenchymal stem cells. Generation of clinical grade dendritic cells is described in the following papers which are incorporated by reference [2-126].

Mesenchymal stem cells (MSCs) are adult stem cells with self-renewing abilities[127] and have been shown to differentiate into a wide range of tissues including mesoderm- and nonmesoderm-derived[127, 128], such as hepatocytes[129-134]. MSCs are capable of entering and maintaining satellite cell niches, particularly in hematopoiesis[135, 136], and are key in tissue repair and regeneration, aging, and regulating homeostasis[137-140]. In the case of heart failure, MSCs can aid in regeneration of renal tissue[141-147], and their interactions with the immune system[148-154] have potential as adjuvants during organ transplants[155], including renal transplantation[156].

MSCs were discovered in 1970 by Friedenstein et al[157] who demonstrated that bone marrow (BM) contained both hematopoietic stem cells (HSCs), which are non-plastic adherent, and a population of a more rare adherent cell. The adherent cells were able to form single cell colonies and were referred to as stromal cells. Those stromal cells, which are capable of self-renewal and expansion in culture are now referred to as mesenchymal stem cells (MSCs). Friedenstein was the first to show that MSCs could differentiate into mesoderm and to demonstrate their importance in controlling the hematopoietic niche [158].

In the 1980s, more research on MSCs found that they could differentiate into muscle, cartilage, bone and adipose-derived cells [159]. Caplan et al showed that MSCs are responsible for bone and cartilage regeneration induced by local cuing and genetic potential[160].

In the 1990s, Pittenger et al isolated MSCs from bone marrow and found that they retained their multilineage potential after expanding into selectively differentiated adipocytic, chondrocytic, or osteocytic lineages[128]. Likewise, Kopen et al showed that bone marrow MSCs differentiated into neural cells when exposed to the brain microenvironment[161]. In 1999, Petersen et al found that bone marrow-derived stem cells could be a source of hepatic oval cells in a rat model[162]. Specifically, they used male to female bone marrow transplant and subsequently induced blockade of hepatocyte proliferation by administration of a hepatotoxin followed by partial hepatectomy. As previously described, this procedure stimulates proliferation of LPC or “reserve cells” which generate new hepatocytes, such cells having previously identified as oval cells. Subsequent to the hepatectomy, Y chromosome, dipeptidyl peptidase IV enzyme, and L21-6 antigen were used to identify the newly generated oval cells, and their hepatocytic progeny to be of bone marrow origin.

The first decade of the 21st century saw a surge of research on MSCs, leading to a greater understanding of their nature and of the cellular process behind regeneration [127, 139, 140]. In 2005, Teratani et al identified growth factors allowing hepatic fate-specification in mice and showed that embryonic stem cells could differentiate into functional hepatocytes [163]. A unique property of MSC is their apparent hypoimmunogenicity and immune modulatory activity [164], which is present in MSC derived from various sources [165]. This is believed to account for the ability to achieve therapeutic effects in an allogeneic manner. Allogeneic bone marrow derived MSC have been used by academic investigators with clinical benefit treatment of diseases such as graft versus host (GVHD) [166-171], osteogenesis imperfecta [172], Hurler syndrome, metachromatic leukodystrophy [173], and acceleration of hematopoietic stem cell engraftment [174-176]. The company Athersys has successfully completed Phase I safety studies using allogeneic bone marrow MSCs is now in efficacy finding clinical trials (Phase II and Phase III) for Multiple Sclerosis, Crohn's Disease, and Graft Versus Host Disease using allogeneic bone marrow derived MSC. Intravenous administration of allogeneic MSCs by Osiris was also reported to induce a statistically significant improvement in renal function in a double-blind study [177].

Currently there several MSC-based therapies that have received governmental approvals including Prochymal™ which was registered in Canada and New Zealand for treatment of graft versus host disease [178, 179]. Although in terms of clinical translation bone marrow MSC are the most advanced, several other sources of MSC are known which possess various properties that may be useful for specific conditions. Bone marrow is also a source for hematopoietic stem cells (HSCs), which have also been used for renal regeneration. Likewise, human placenta is an easily accessible source of abundant MSCs, which can be differentiated in vitro. Finally, MSCs with tissue regenerative abilities can also be isolated from adipose tissue and induced to hepatocytes in large numbers.

In some embodiments of the invention reprogrammed immune cells are administered with MSC for treatment of heart failure. The discussion below provides examples of the use of MSC in heart failure, which may be useful for one of skill in the art to combine MSC with reprogramemd immune cells

Methods of deriving cord tissue mesenchymal stem cells from human umbilical tissue are provided. The cells are capable of self-renewal and expansion in culture, and have the potential to differentiate into cells of other phenotypes. The method comprises (a) obtaining human umbilical tissue; (b) removing substantially all of blood to yield a substantially blood-free umbilical tissue, (c) dissociating the tissue by mechanical or enzymatic treatment, or both, (d) resuspending the tissue in a culture medium, and (e) providing growth conditions which allow for the growth of a human umbilicus-derived cell capable of self-renewal and expansion in culture and having the potential to differentiate into cells of other phenotypes. Tissue can be obtained from any completed pregnancy, term or less than term, whether delivered vaginally, or through other routes, for example surgical Cesarean section. Obtaining tissue from tissue banks is also considered within the scope of the present invention.

The tissue is rendered substantially free of blood by any means known in the art. For example, the blood can be physically removed by washing, rinsing, and diluting and the like, before or after bulk blood removal for example by suctioning or draining. Other means of obtaining a tissue substantially free of blood cells might include enzymatic or chemical treatment.

Dissociation of the umbilical tissues can be accomplished by any of the various techniques known in the art, including by mechanical disruption, for example, tissue can be aseptically cut with scissors, or a scalpel, or such tissue can be otherwise minced, blended, ground, or homogenized in any manner that is compatible with recovering intact or viable cells from human tissue.

In a presently preferred embodiment, the isolation procedure also utilizes an enzymatic digestion process. Many enzymes are known in the art to be useful for the isolation of individual cells from complex tissue matrices to facilitate growth in culture. As discussed above, a broad range of digestive enzymes for use in cell isolation from tissue is available to the skilled artisan. Ranging from weakly digestive (e.g. deoxyribonucleases and the neutral protease, dispase) to strongly digestive (e.g. papain and trypsin), such enzymes are available commercially. A nonexhaustive list of enzymes compatable herewith includes mucolytic enzyme activities, metalloproteases, neutral proteases, serine proteases (such as trypsin, chymotrypsin, or elastase), and deoxyribonucleases. Presently preferred are enzyme activites selected from metalloproteases, neutral proteases and mucolytic activities. For example, collagenases are known to be useful for isolating various cells from tissues. Deoxyribonucleases can digest single-stranded DNA and can minimize cell-clumping during isolation. Enzymes can be used alone or in combination. Serine protease are preferably used in a sequence following the use of other enzymes as they may degrade the other enzymes being used. The temperature and time of contact with serine proteases must be monitored. Serine proteases may be inhibited with alpha 2 microglobulin in serum and therefore the medium used for digestion is preferably serum-free. EDTA and DNase are commonly used and may improve yields or efficiencies. Preferred methods involve enzymatic treatment with for example collagenase and dispase, or collagenase, dispase, and hyaluronidase, and such methods are provided wherein in certain preferred embodiments, a mixture of collagenase and the neutral protease dispase are used in the dissociating step. More preferred are those methods which employ digestion in the presence of at least one collagenase from Clostridium histolyticum, and either of the protease activities, dispase and thermolysin. Still more preferred are methods employing digestion with both collagenase and dispase enzyme activities. Also preferred are methods which include digestion with a hyaluronidase activity in addition to collagenase and dispase activities. The skilled artisan will appreciate that many such enzyme treatments are known in the art for isolating cells from various tissue sources. For example, the LIBERASE BLENDZYME (Roche) series of enzyme combinations of collagenase and neutral protease are very useful and may be used in the instant methods. Other sources of enzymes are known, and the skilled artisan may also obtain such enzymes directly from their natural sources. The skilled artisan is also well-equipped to assess new, or additional enzymes or enzyme combinations for their utility in isolating the cells of the invention. Preferred enzyme treatments are 0.5, 1, 1.5, or 2 hours long or longer. In other preferred embodiments, the tissue is incubated at 37.degree. C. during the enzyme treatment of the dissociation step. Diluting the digest may also improve yields of cells as cells may be trapped within a viscous digest.

While the use of enzyme activites is presently preferred, it is not required for isolation methods as provided herein. Methods based on mechanical separation alone may be successful in isolating the instant cells from the umbilicus as discussed above.

The cells can be resuspended after the tissue is dissociated into any culture medium as discussed herein above. Cells may be resuspended following a centrifugation step to separate out the cells from tissue or other debris. Resuspension may involve mechanical methods of resuspending, or simply the addition of culture medium to the cells.

Providing the growth conditions allows for a wide range of options as to culture medium, supplements, atmospheric conditions, and relative humidity for the cells. A preferred temperature is 37.degree. C., however the temperature may range from about 35.degree. C. to 39.degree. C. depending on the other culture conditions and desired use of the cells or culture.

Presently preferred are methods which provide cells which require no exogenous growth factors, except as are available in the supplemental serum provided with the Growth Medium. Also provided herein are methods of deriving umbilical cells capable of expansion in the absence of particular growth factors. The methods are similar to the method above, however they require that the particular growth factors (for which the cells have no requirement) be absent in the culture medium in which the cells are ultimately resuspended and grown in. In this sense, the method is selective for those cells capable of division in the absence of the particular growth factors. Preferred cells in some embodiments are capable of growth and expansion in chemically-defined growth media with no serum added. In such cases, the cells may require certain growth factors, which can be added to the medium to support and sustain the cells. Presently preferred factors to be added for growth on serum-free media include one or more of FGF, EGF, IGF, and PDGF. In more preferred embodiments, two, three or all four of the factors are add to serum free or chemically defined media. In other embodiments, LIF is added to serum-free medium to support or improve growth of the cells.

Also provided are methods wherein the cells can expand in the presence of from about 5% to about 20% oxygen in their atmosphere. Methods to obtain cells that require L-valine require that cells be cultured in the presence of L-valine. After a cell is obtained, its need for L-valine can be tested and confirmed by growing on D-valine containing medium that lacks the L-isomer.

Methods are provided wherein the cells can undergo at least 25, 30, 35, or 40 doublings prior to reaching a senescent state. Methods for deriving cells capable of doubling to reach 10.sup.14 cells or more are provided. Preferred are those methods which derive cells that can double sufficiently to produce at least about 10.sup.14, 10.sup.15, 10.sup.16, or 10.sup.17 or more cells when seeded at from about 10.sup.3 to about 10.sup.6 cells/cm.sup.2 in culture. Preferably these cell numbers are produced within 80, 70, or 60 days or less. In one embodiment, cord tissue mesenchymal stem cells are isolated and expanded, and possess one or more markers selected from a group comprising of CD10, CD13, CD44, CD73, CD90, CD141, PDGFr-alpha, or HLA-A,B,C. In addition, the cells do not produce one or more of CD31, CD34, CD45, CD117, CD141, or HLA-DR,DP, DQ.

In one embodiment, bone marrow MSC lots are generated, means of generating BM MSC are known in the literature and examples are incorporated by reference.

In one embodiment BM-MSC are generated as follows
1. 500 mL Isolation Buffer is prepared (PBS+2% FBS+2 mM EDTA) using sterile components or filtering Isolation Buffer through a 0.2 micron filter. Once made, the Isolation Buffer was stored at 2-8.degree. C.
2. The total number of nucleated cells in the BM sample is counted by taking 10 .mu.L BM and diluting it 1/50-1/100 with 3% Acetic Acid with Methylene Blue (STEMCELL Catalog #07060). Cells are counted using a hemacytometer.
3. 50 mL Isolation Buffer is warmed to room temperature for 20 minutes prior to use and bone marrow was diluted 5/14 final dilution with room temperature Isolation Buffer (e.g. 25 mL BM was diluted with 45 mL Isolation Buffer for a total volume of 70 mL).
4. In three 50 mL conical tubes (BD Catalog #352070), 17 mL Ficoll-Paque™ PLUS (Catalog #07907/07957) is pipetted into each tube. About 23 mL of the diluted BM from step 3 was carefully layered on top of the Ficoll-Paque™ PLUS in each tube.
5. The tubes are centrifuged at room temperature (15-25.degree. C.) for 30 minutes at 300.times.g in a bench top centrifuge with the brake off.
6. The upper plasma layer is removed and discarded without disturbing the plasma:Ficoll-Paque™ PLUS interface. The mononuclear cells located at the interface layer are carefully removed and placed in a new 50 mL conical tube. Mononuclear cells are resuspended with 40 mL cold (2-8.degree. C.) Isolation Buffer and mixed gently by pipetting.
7. Cells were centrifuged at 300.times.g for 10 minutes at room temperature in a bench top centrifuge with the brake on. The supernatant is removed and the cell pellet resuspended in 1-2 mL cold Isolation Buffer.
8. Cells were diluted 1/50 in 3% Acetic Acid with Methylene Blue and the total number of nucleated cells counted using a hemacytometer.
9. Cells are diluted in Complete Human MesenCult®-Proliferation medium (STEMCELL catalog #05411) at a final concentration of 1.times.10.sup.6 cells/mL.
10. BM-derived cells were ready for expansion and CFU-F assays in the presence of GW2580, which can then be used for specific applications.

In one embodiment, MSC are generated according to protocols previously utilized for treatment of patients utilizing bone marrow derived MSC. Specifically, bone marrow is aspirated (10-30 ml) under local anesthesia (with or without sedation) from the posterior iliac crest, collected into sodium heparin containing tubes and transferred to a Good Manufacturing Practices (GMP) clean room. Bone marrow cells are washed with a washing solution such as Dulbecco's phosphate-buffered saline (DPBS), RPMI, or PBS supplemented with autologous patient plasma and layered on to 25 ml of Percoll (1.073 g/ml) at a concentration of approximately 1-2′107 cells/ml. Subsequently the cells are centrifuged at 900 g for approximately 30 min or a time period sufficient to achieve separation of mononuclear cells from debris and erythrocytes. Said cells are then washed with PBS and plated at a density of approximately 1′106 cells per ml in 175 cm2 tissue culture flasks in DMEM with 10% FCS with flasks subsequently being loaded with a minimum of 30 million bone marrow mononuclear cells. The MSCs are allowed to adhere for 72 h followed by media changes every 3-4 days. Adherent cells are removed with 0.05% trypsin-EDTA and replated at a density of 1′106 per 175 cm2. Said bone marrow MSC may be administered intravenously, or in a preferred embodiment, intrathecally in a patient suffering radiation associated neurodegenerative manifestations. Although doses may be determined by one of skill in the art, and are dependent on various patient characteristics, intravenous administration may be performed at concentrations ranging from 1-10 million MSC per kilogram, with a preferred dose of approximately 2-5 million cells per kilogram.

In order to determine the quality of MSC cultures, flow cytometry is performed on all cultures for surface expression of SH-2, SH-3, SH-4 MSC markers and lack of contaminating CD14- and CD-45 positive cells. Cells were detached with 0.05% trypsin-EDTA, washed with DPBS+2% bovine albumin, fixed in 1% paraformaldehyde, blocked in 10% serum, incubated separately with primary SH-2, SH-3 and SH-4 antibodies followed by PE-conjugated anti-mouse IgG(H+L) antibody. Confluent MSC in 175 cm2 flasks are washed with Tyrode's salt solution, incubated with medium 199 (M199) for 60 min, and detached with 0.05% trypsin-EDTA (Gibco). Cells from 10 flasks were detached at a time and MSCs were resuspended in 40 ml of M199+1% human serum albumin (HSA; American Red Cross, Washington D.C., USA). MSCs harvested from each 10-flask set were stored for up to 4 h at 4° C. and combined at the end of the harvest. A total of 2-10′106 MSC/kg were resuspended in M199+1% HSA and centrifuged at 460 g for 10 min at 20° C. Cell pellets were resuspended in fresh M199+1% HSA media and centrifuged at 460 g for 10 min at 20° C. for three additional times. Total harvest time was 2-4 h based on MSC yield per flask and the target dose. Harvested MSC were cryopreserved in Cryocyte (Baxter, Deerfield, Ill., USA) freezing bags using a rate controlled freezer at a final concentration of 10% DMSO (Research Industries, Salt Lake City, Utah, USA) and 5% HSA. On the day of infusion cryopreserved units were thawed at the bedside in a 37° C. water bath and transferred into 60 ml syringes within 5 min and infused intravenously into patients over 10-15 min. Patients are premedicated with 325-650 mg acetaminophen and 12.5-25 mg of diphenhydramine orally. Blood pressure, pulse, respiratory rate, temperature and oxygen saturation are monitored at the time of infusion and every 15 min thereafter for 3 h followed by every 2 h for 6 h.

Within the context of the invention, exosomes and microparticles may be used interchangeably. Exosomes from MSC may be generated from a mesenchymal stem cell conditioned medium (MSC-CM). Said exosomes are used in the context of the invention to reprogram immunocytes ex vivo or in vivo. Said particle may be isolated for example by being separated from non-associated components based on any property of the particle. For example, the particle may be isolated based on molecular weight, size, shape, composition or biological activity. The conditioned medium may be filtered or concentrated or both during, prior to or subsequent to separation. For example, it may be filtered through a membrane, for example one with a size or molecular weight cut-off. It may be subject to tangential force filtration or ultrafiltration. Filtration of conditioned media is described in the following and incorporated by reference [180]. For example, filtration with a membrane of a suitable molecular weight or size cutoff. The conditioned medium, optionally filtered or concentrated or both, may be subject to further separation means, such as column chromatography. For example, high performance liquid chromatography (HPLC) with various columns may be used. The columns may be size exclusion columns or binding columns. One or more properties or biological activities of the particle may be used to track its activity during fractionation of the mesenchymal stem cell conditioned medium (MSC-CM). As an example, light scattering, refractive index, dynamic light scattering or UV-visible detectors may be used to follow the particles. For example, a therapeutic activity such as antirheumatic activity may be used to track the activity during fractionation. In one embodiment antirheumatic activity is assessed by ability to inhibit TNF-alpha production from stimulated monocytes or monocytic lineage cell such as macrophages or dendritic cells.

In one aspect of the invention MSC are cultured for about 1, 2, 3, 4, 5, 6, 7, 8, 9, 10 days or more, for example 3 days. The conditioned medium may be obtained by separating the cells from the medium. The conditioned medium may be centrifuged, for example at 500 g. it may be concentrated by filtration through a membrane. The membrane may comprise a >1000 kDa membrame. The conditioned medium may be concentrated about 50 times or more. The conditioned medium may be subject to liquid chromatography such as HPLC. The conditioned medium may be separated by size exclusion. Any size exclusion matrix such as Sepharose may be used. As an example, a TSK Guard column SWXL, 6.times.40 mm or a TSK gel G4000 SWXL, 7.8.times.300 mm may be employed. The eluent buffer may comprise any physiological medium such as saline. It may comprise 20 mM phosphate buffer with 150 mM of NaCl at pH 7.2. The chromatography system may be equilibrated at a flow rate of 0.5 ml/min. The elution mode may be isocratic. UV absorbance at 220 nm may be used to track the progress of elution. Fractions may be examined for dynamic light scattering (DLS) using a quasi-elastic light scattering (QELS) detector. Fractions which are found to exhibit dynamic light scattering may be retained. For example, a fraction which is produced by the general method as described above, and which elutes with a retention time of 11-13 minutes, such as 12 minutes, is found to exhibit dynamic light scattering. The r.sub.h of particles in this peak is about 45-55 nm. Such fractions comprise mesenchymal stem cell particles such as exosomes.

MSC can be prepared from a variety of tissues, such as bone marrow cells [181-187], umbilical cord tissue [188-190], peripheral blood [191-193], amniotic membrane [194], amniotic fluid, mobilized peripheral blood [195], adipose tissue [196, 197], endometrium and other tissues. When tissue sources of MSC are used said tissue isolates from which the Reprogrammed immune cells are isolated comprise a mixed populations of cells. Reprogrammed immune cells constitute a very small percentage in these initial populations. They must be purified away from the other cells before they can be expanded in culture sufficiently to obtain enough cells for therapeutic applications.

The choice of formulation for administering reprogrammed immune cells for a given application will depend on a variety of factors. Prominent among these will be the species of subject, the nature of the disorder, dysfunction, or disease being treated and its state and distribution in the subject, the nature of other therapies and agents that are being administered, the optimum route for administration of the reprogrammed immune cells, survivability of reprogrammed immune cells via the route, the dosing regimen, and other factors that will be apparent to those skilled in the art. In particular, for instance, the choice of suitable carriers and other additives will depend on the exact route of administration and the nature of the particular dosage form, for example, liquid dosage form (e.g., whether the composition is to be formulated into a solution, a suspension, gel or another liquid form, such as a time release form or liquid-filled form).

For example, cell survival can be an important determinant of the efficacy of cell-based therapies. This is true for both primary and adjunctive therapies. Another concern arises when target sites are inhospitable to cell seeding and cell growth. This may impede access to the site and/or engraftment there of therapeutic Reprogrammed immune cells. Various embodiments of the invention comprise measures to increase cell survival and/or to overcome problems posed by barriers to seeding and/or growth.

Examples of compositions comprising reprogrammed immune cells include liquid preparations, including suspensions and preparations for intramuscular or intravenous administration (e.g., injectable administration), such as sterile suspensions or emulsions. Such compositions may comprise an admixture of Reprogrammed immune cells with a suitable carrier, diluent, or excipient such as sterile water, physiological saline, glucose, dextrose, or the like. The compositions can also be lyophilized. The compositions can contain auxiliary substances such as wetting or emulsifying agents, pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE,” 17th edition, 1985, incorporated herein by reference, may be consulted to prepare suitable preparations, without undue experimentation.

Compositions of the invention often are conveniently provided as liquid preparations, e.g., isotonic aqueous solutions, suspensions, emulsions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues.

Various additives often will be included to enhance the stability, sterility, and isotonicity of the compositions, such as antimicrobial preservatives, antioxidants, chelating agents, and buffers, among others. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents that delay absorption, for example, aluminum monostearate, and gelatin. According to the present invention, however, any vehicle, diluent, or additive used would have to be compatible with the cells.

Reprogrammed immune cells solutions, suspensions, and gels normally contain a major amount of water (preferably purified, sterilized water) in addition to the cells. Minor amounts of other ingredients such as pH adjusters (e.g., a base such as NaOH), emulsifiers or dispersing agents, buffering agents, preservatives, wetting agents and jelling agents (e.g., methylcellulose) may also be present. In some embodiments of the invention, treatment of viral associated renal damage is disclosed using immune cells that have been reprogrammed with regenerative cells. Some patients hospitalized for COVID-19 have had increased levels of renal enzyme or markers that indicate their hearts are at least temporarily damaged. Also, renal damage is more common in patients who have severe COVID-19 disease.

Typically, the compositions will be isotonic, i.e., they will have the same osmotic pressure as blood and lacrimal fluid when properly prepared for administration.

The desired isotonicity of the compositions of this invention may be accomplished using sodium chloride, or other pharmaceutically acceptable agents such as dextrose, boric acid, sodium tartrate, propylene glycol, or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount, which will achieve the selected viscosity. Viscous compositions are normally prepared from solutions by the addition of such thickening agents.

A pharmaceutically acceptable preservative or cell stabilizer can be employed to increase the life of reprogrammed immune cells compositions. If such preservatives are included, it is well within the purview of the skilled artisan to select compositions that will not affect the viability or efficacy of the reprogrammed immune cells.

Those skilled in the art will recognize that the components of the compositions should be chemically inert. This will present no problem to those skilled in chemical and pharmaceutical principles. Problems can be readily avoided by reference to standard texts or by simple experiments (not involving undue experimentation) using information provided by the disclosure, the documents cited herein, and generally available in the art.

Sterile injectable solutions can be prepared by incorporating the cells utilized in practicing the present invention in the required amount of the appropriate solvent with various amounts of the other ingredients, as desired.

In some embodiments, reprogrammed immune cells are formulated in a unit dosage injectable form, such as a solution, suspension, or emulsion. Pharmaceutical formulations suitable for injection of Reprogrammed immune cells typically are sterile aqueous solutions and dispersions. Carriers for injectable formulations can be a solvent or dispersing medium containing, for example, water, saline, phosphate buffered saline, polyol (for example, glycerol, propylene glycol, liquid polyethylene glycol, and the like), and suitable mixtures thereof.

The skilled artisan can readily determine the amount of cells and optional additives, vehicles, and/or carrier in compositions to be administered in methods of the invention. Typically, any additives (in addition to the cells) are present in an amount of 0.001 to 50 wt % in solution, such as in phosphate buffered saline. The active ingredient is present in the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %.

For any composition to be administered to an animal or human, and for any particular method of administration, it is preferred to determine therefore: toxicity, such as by determining the lethal dose (LD) and LD50 in a suitable animal model, e.g., rodent such as mouse or rat; and, the dosage of the composition(s), concentration of components therein, and timing of administering the composition(s), which elicit a suitable response. Such determinations do not require undue experimentation from the knowledge of the skilled artisan, this disclosure, and the documents cited herein. And, the time for sequential administrations can be ascertained without undue experimentation.

In some embodiments Reprogrammed immune cells are encapsulated for administration, particularly where encapsulation enhances the effectiveness of the therapy, or provides advantages in handling and/or shelf life. Encapsulation in some embodiments where it increases the efficacy of the cell mediated immunosuppression may, as a result, also reduce the need for immunosuppressive drug therapy.

Also, encapsulation in some embodiments provides a barrier to a subject's immune system that may further reduce a subject's immune response to the Reprogrammed immune cells (which generally are not immunogenic or are only weakly immunogenic in allogeneic transplants), thereby reducing any graft rejection or inflammation that might occur upon administration of the cells.

In a variety of embodiments where reprogrammed immune cells are administered in admixture with cells of another type, which are more typically immunogenic in an allogeneic or xenogeneic setting, encapsulation may reduce or eliminate adverse host immune responses to the non-reprogrammed immune cells cells and/or GVHD that might occur in an immunocompromised host if the admixed cells are immunocompetent and recognize the host as non-self.

Reprogrammed immune cells may be encapsulated by membranes, as well as capsules, prior to implantation. It is contemplated that any of the many methods of cell encapsulation available may be employed. In some embodiments, cells are individually encapsulated. In some embodiments, many cells are encapsulated within the same membrane. In embodiments in which the cells are to be removed following implantation, a relatively large size structure encapsulating many cells, such as within a single membrane, may provide a convenient means for retrieval.

A wide variety of materials may be used in various embodiments for microencapsulation of Reprogrammed immune cells. Such materials include, for example, polymer capsules, alginate-poly-L-lysine-alginate microcapsules, barium poly-L-lysine alginate capsules, barium alginate capsules, polyacrylonitrile/polyvinylchloride (PAN/PVC) hollow fibers, and polyethersulfone (PES) hollow fibers.

Techniques for microencapsulation of cells that may be used for administration of Reprogrammed immune cells are known to those of skill in the art and are described, for example, in Chang, P., et al., 1999; Matthew, H. W., et al., 1991; Yanagi, K., et al., 1989; Cal Z. H., et al., 1988; Chang, T. M., 1992 and in U.S. Pat. No. 5,639,275 (which, for example, describes a biocompatible capsule for long-term maintenance of cells that stably express biologically active molecules. Additional methods of encapsulation are in European Patent Publication No. 301,777 and U.S. Pat. Nos. 4,353,888; 4,744,933; 4,749,620; 4,814,274; 5,084,350; 5,089,272; 5,578,442; 5,639,275; and 5,676,943. All of the foregoing are incorporated herein by reference in parts pertinent to encapsulation of Reprogrammed immune cells.

Certain embodiments incorporate Reprogrammed immune cells into a polymer, such as a biopolymer or synthetic polymer. Examples of biopolymers include, but are not limited to, fibronectin, fibin, fibrinogen, thrombin, collagen, and proteoglycans. Other factors, such as the cytokines discussed above, can also be incorporated into the polymer. In other embodiments of the invention, Reprogrammed immune cells may be incorporated in the interstices of a three-dimensional gel. A large polymer or gel, typically, will be surgically implanted. A polymer or gel that can be formulated in small enough particles or fibers can be administered by other common, more convenient, non-surgical routes.

Pharmaceutical compositions of the invention may be prepared in many forms that include tablets, hard or soft gelatin capsules, aqueous solutions, suspensions, and liposomes and other slow-release formulations, such as shaped polymeric gels. Oral liquid pharmaceutical compositions may be in the form of, for example, aqueous or oily suspensions, solutions, emulsions, syrups, or elixirs, or may be presented as a dry product for constitution with water or other suitable vehicle before use. Such liquid pharmaceutical compositions may contain conventional additives such as suspending agents, emulsifying agents, non-aqueous vehicles (which may include edible oils), or preservatives. An oral dosage form may be formulated such that cells are released into the intestine after passing through the stomach. Such formulations are described in U.S. Pat. No. 6,306,434 and in the references contained therein.

Pharmaceutical compositions suitable for rectal administration can be prepared as unit dose suppositories. Suitable carriers include saline solution and other materials commonly used in the art.

For administration by inhalation, cells can be conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount.

Alternatively, for administration by inhalation or insufflation, a means may take the form of a dry powder composition, for example, a powder mix of a modulator and a suitable powder base such as lactose or starch. The powder composition may be presented in unit dosage form in, for example, capsules or cartridges or, e.g., gelatin or blister packs from which the powder may be administered with the aid of an inhalator or insufflator. For intra-nasal administration, cells may be administered via a liquid spray, such as via a plastic bottle atomizer.

Reprogrammed immune cells may be administered with other pharmaceutically active agents. In some embodiments one or more of such agents are formulated together with Reprogrammed immune cells for administration. In some embodiments the Reprogrammed immune cells and the one or more agents are in separate formulations. In some embodiments the compositions comprising the Reprogrammed immune cells and/or the one or more agents are formulated with regard to adjunctive use with one another.

Reprogrammed immune cells may be administered in a formulation comprising a immunosuppressive agents, such as any combination of any number of a corticosteroid, cyclosporin A, a cyclosporin-like immunosuppressive agent, cyclophosphamide, antithymocyte globulin, azathioprine, rapamycin, FK-506, and a macrolide-like immunosuppressive agent other than FK-506 and rapamycin. In certain embodiments, such agents include a corticosteroid, cyclosporin A, azathioprine, cyclophosphamide, rapamycin, and/or FK-506. Immunosuppressive agents in accordance with the foregoing may be the only such additional agents or may be combined with other agents, such as other agents noted herein. Other immunosuppressive agents include Tacrolimus, Mycophenolate mofetil, and Sirolimus.

Certain embodiments of the present disclosure concern the treatment or prevention of one or more kidney diseases in an individual. As used herein, “kidney disease” may refer to any condition that reduces the normal function of the kidney and/or changes the physiology of the kidney. The normal function of the kidney comprises at least: regulation of fluid volume, regulation of osmolarity, regulation of ion concentrations (such as salt concentrations), regulation of physiological pH, filtration of wastes and toxins from the blood, production of hormones, generation of urine, regulation of blood pressure, or a combination thereof.

Reduction in normal kidney function may or may not be monitored by any means known in the art, including measuring certain biomarkers either in blood samples or urine samples from an individual of the present disclosure. Such biomarkers include, but are not limited to, creatinine, albumin, blood urea nitrogen (BUN), cystatin c, beta-trace protein (BTP), podocalyxin, nephrin, alpha 1-microglobulin, beta 2-microglobulin, glutathione s-transferase, interleukin-18, kidney injury molecule-1 (KIM-1), liver-type fatty acid-binding protein, netrin-1, neutrophil gelatinase-associated lipocalcin (NGAL), n-acetyl-beta-d-glucosaminidase (NAG), sodium, potassium, calcium, bicarbonate, pH, red blood cell count, lymphocytes, eosinophils, casts, ADAMTS13, ANCA, heavy chain immunoglobulins, light chain immunoglobulins, or a combination thereof. Changes in levels of the biomarkers from a baseline or measurements outside of a normal range, as defined by the art, may indicate reduction in normal kidney function. Reduction in normal kidney function may be measured by calculating the glomerular filtration rate (GFR), estimated GFR (eGFR), urine albumin to creatinine ratio (ACR), urine protein to creatinine ratio (uPCR), total urine protein (including 24 hour urine protein). Changes in measurements from a baseline or measurements outside of a normal range, as defined by the art, may indicate reduction in normal kidney function. Changes in physiology of the kidney may be measured by imaging the kidney, such as by ultrasound and/or CT scan. A biopsy of the kidney may or may not be taken to assess for signs of one or more kidney diseases.

The disease may affect one or both kidneys in an individual and may cause symptoms in the individual or be asymptomatic. The kidney disease may be mediated by any cause including being immune-mediated, infection-mediated, metabolism-mediated, hormone-mediated, genetically-mediated, or mediated by any other biological process.

The kidney disease of the present disclosure may be directly or indirectly related to exposure, accidentally or intentionally, of one or more nephrotoxic compositions or agent. Nephrotoxic compositions or agents include, but are not limited to, chemotherapies, radiation, immune-modulating compositions or agents, non-steroid anti-inflammatory drugs (NSAIDs), antibiotics, antifungals, antivirals, diuretics, beta blockers, ACE inhibitors, vasodilators, cyclosporins, steroids, narcotics, or any other composition and/or agent that may induce nephrotoxicity.

In certain embodiments, the kidney disease of the present disclosure may be acute kidney injury, acute proliferative glomerulonephritis, adenine phosphoribosyltransferase deficiency, alabama rot, Alport syndrome, analgesic nephropathy, autosomal dominant polycystic kidney disease, autosomal recessive polycystic kidney disease, balkan endemic nephropathy, bardoxolone methyl, benign nephrosclerosis, bright's disease, cardiorenal syndrome, CFHR5 nephropathy, chronic kidney disease, chronic kidney disease-mineral and bone disorder, congenital nephrotic syndrome, conorenal syndrome, contrast-induced nephropathy, cystic kidney disease, dent's disease, diabetic nephropathy, diffuse proliferative nephritis, distal renal tubular acidosis, diuresis, EAST syndrome, end stage renal disease, Epstein syndrome, Fanconi syndrome, fechtner syndrome, focal proliferative nephritis, focal segmental glomerulosclerosis, fraley syndrome, galloway mowat syndrome, gitelman syndrome, glomerulocystic kidney disease, glomerulopathy, goodpasture syndrome, hematologic diseases information service, high anion gap metabolic acidosis, HIV-associated nephropathy, horseshoe kidney, hydronephrosis, hyperkalemia, hypernatremia, hypertensive kidney disease, hyponatremia, IgA nephropathy, interstitial nephritis, juvenile nephronophthisis, kidney cancer, kidney stone disease, Lightwood-Albright syndrome, lupus nephritis, malarial nephropathy, medullary cystic kidney disease, medullary sponge kidney, membranous glomerulonephritis, mesoamerican nephropathy, milk-alkali syndrome, minimal mesangial glomerulonephritis, multicystic dysplastic kidney, nephritis, nephrocalcinosis, nephrogenic diabetes insipidus, nephromegaly, nephroptosis, nephrosis, nephrotic syndrome, nutcracker syndrome, papillorenal syndrome, phosphate nephropathy, polycystic kidney disease, primary hyperoxaluria, proximal renal tubular acidosis, pyelonephritis, pyonephrosis, rapidly progressive glomerulonephritis, renal agenesis, renal angina, renal artery stenosis, renal cyst, renal ischemia, renal osteodystrophy, renal papillary necrosis, renal tubular acidosis, renal vein thrombosis, secondary hypertension, serpentine fibula-polycystic kidney syndrome, shunt nephritis, sickle cell nephropathy, tetracapsuloides, thin basement membrane disease, transplant glomerulopathy, tubulointerstitial nephritis and uveitis, tubulopathy, uremia, uremic frost, wunderlich syndrome, or a combination thereof.

III. Therapeutic or Preventative Administration

Certain embodiments of the present disclosure concern the administration of an effective amount of regenerative cells to an individual having, or at risk of having, kidney disease, including any kidney disease or loss of kidney function described herein. The regenerative cells may be administered in any suitable manner, including intra-arterially and/or intravenously, merely as examples. In some embodiments, the regenerative cells are administered to an individual of the present disclosure therapeutically or prophylactically. The regenerative cells may be administered to the individual at the onset or initial diagnosis of the kidney disease. The regenerative cells may be administered at any time before or after the onset or initial diagnosis of the kidney disease. The regenerative cells may be administered less than 24 hours or less than 48 hours after the onset or diagnosis of the kidney disease. The regenerative cells may be administered more than 24 hours or more than 48 hours after the onset or diagnosis of the kidney disease. The regenerative cells may be administered between 24 to 48 hours after the onset or diagnosis of the kidney disease. The regenerative cells may be administered approximately 24, 26, 28, 30, 32, 34, 36, 38, 40, 42, 44, 46, or 48 hours after the onset or diagnosis of the kidney disease.

In some embodiments, an individual of the present disclosure is administered a prophylactically or therapeutically effective amount of regenerative cells of the present disclosure. The prophylactically or therapeutically effective amount of regenerative cells may be between approximately 7×105 cells/kg of individual's body weight and approximately 7×106 cells/kg of individual's body weight. In some embodiments, the prophylactically or therapeutically effective amount of regenerative cells is between about approximately 2×106 cells/kg of individual's body weight and approximately 5×106 cells/kg of individual's body weight. In some embodiments, the prophylactically or therapeutically effective amount of regenerative cells approximately 2×106 cells/kg of individual's body weight.

Morigi et al used a model of renal injury induced in mice by the anticancer agent cisplatin was chosen. Injection of mesenchymal stem cells of male bone marrow origin remarkably protected cisplatin-treated syngeneic female mice from renal function impairment and severe tubular injury. Y chromosome-containing cells localized in the context of the tubular epithelial lining and displayed binding sites for Lens culinaris lectin, indicating that mesenchymal stem cells engraft the damaged kidney and differentiate into tubular epithelial cells, thereby restoring renal structure and function. Mesenchymal stem cells markedly accelerated tubular proliferation in response to cisplatin-induced damage, as revealed by higher numbers of Ki-67-positive cells within the tubuli with respect to cisplatin-treated mice that were given saline [4]. Hematopoietic stem cells failed to exert beneficial effects. In the context of the current disclosure, in one embodiment, regenerative cells are used to induce de novo differentiation of cells into tubular epithelial cells, as well as to protect from chemotherapy induced kidney damage.

In another study, the effect of mesenchymal stem cell (MSCs) infusion on the recovery from ARF induced by intramuscle injection of glycerol in C57/BL6 mice was assessed. In this model, ARF is associated with an extensive necrosis of tubular epithelial cells due to myoglobin- and hemoglobin-induced injury. MSCs were obtained from bone marrow of transgenic mice expressing green fluorescent protein (GFP). MSC GFP-positive cells (MSC-GFP(+)) injected intravenously homed to the kidney of mice with glycerol-induced ARF but not to the kidney of normal mice. MSC-GFP(+) localized in the context of the tubular epithelial lining and expressed cytokeratin, indicating that MSCs engrafted in the damaged kidney, differentiated into tubular epithelial cells and promoted the recovery of morphological and functional alterations. Moreover, MSCs enhanced tubular proliferation as detected by the increased number of proliferating cell nuclear antigen (PCNA) positive cells. A significant contribution of the engrafted MSCs in the regeneration of tubular epithelial cells was shown by the presence of a consistent number of GFP(+) tubular cells 21 days after the induction of injury [5].

Similarly to MSCs, regenerative cells of the disclosure are capable of treating kidney damage both through direct differentiation inducing mechanisms, as well as through indirect mechanisms. Indirect mechanisms include protection from apoptosis, production of growth factors that stimulate endogenous progenitors, and reduction of fibrosis. In one study, intracarotid administration of MSC (approximately 106/animal) either immediately or 24 h after renal ischemia resulted in significantly improved renal function, higher proliferative and lower apoptotic indexes, as well as lower renal injury and unchanged leukocyte infiltration scores. Such renoprotection was not obtained with syngeneic regenerative cells. Using in vivo two-photon laser confocal microscopy, fluorescence-labeled MSC were detected early after injection in glomeruli, and low numbers attached at microvasculature sites. However, within 3 days of administration, none of the administered MSC had differentiated into a tubular or endothelial cell phenotype. At 24 h after injury, expression of proinflammatory cytokines IL-1beta, TNF-alpha, IFN-gamma, and inducible nitric oxide synthase was significantly reduced and that of anti-inflammatory IL-10 and bFGF, TGF-alpha, and Bcl-2 was highly upregulated in treated kidneys [6].

In another study kidney ischemia reperfusion injury was induced by clamping the bilateral pedicles for 60 minutes. Mesenchymal stem cells (MSC), which had been isolated and cultivated in plastic flasks, were administered to mice 6 hours after injury. Real-time polymerase chain reaction was used to quantify interleukin (IL)-4 and IL-1beta mRNAs. Proliferative nuclear cell antigen (PCNA) was used to calculate tubular regeneration. It was shown that administration of MSC attenuated renal injury; serum creatinine and plasma urea levels were significantly reduced 24 hours after reperfusion. PCNA immunohistochemistry showed that regeneration occurred faster in renal tissues of animals that received MSC than in tissues of control animals. Analyses of cytokine expression in renal tissue demonstrated a greater level of anti-inflammatory cytokines in MSC-treated animals [7]. Others studies regarding use of cellular therapies for treatment of kidney failure are incorporated by reference to provide guidance on dosage and means of administration [8-24].

In some embodiments of the disclosure, correction of endothelial progenitor cell (EPC) deficiency is performed prior to administration of regenerative cells. It is known that patients with chronic renal failure (CRF) have such deficiencies. For example, in one study, EPCs were isolated from CRF patients on maintenance hemodialysis (n=44) and from a normal control group (n=30). CRF patients showed markedly decreased numbers of EPC (44.6%) and colonies (75.3%) when compared with the controls (P<0.001). These findings were corroborated by 30.5% decrease in EPC migratory function in response to vascular endothelial growth factor (VEGF) (P=0.040) and 48.8% decrease in EPC incorporation into human umbilical vein endothelial cells (HUVEC) (P<0.001). In addition, Framingham's risk factor score of both CRF (r=−0.461, P=0.010) and normal group (r=−0.367, P=0.016) significantly correlated with the numbers of EPC. Indeed, the number of circulating EPC was significantly lower in CRF patients than in normal group under the same burden of risk factors (P<0.001). A significant correlation was also observed between dialysis dose (Kt/V) and EPC incorporation into HUVEC (r=0.427, P=0.004) [25].

It is believed that after kidney injury, surviving renal epithelial cells undergo a program of dedifferentiation and take on mesenchymal characteristics [26]. These cells proliferate to restore the integrity of the denuded basement membrane, and subsequently redifferentiate into a functional epithelium [26-32]. The process of dedifferentiation and redifferentiation is dependent on EGF [33], accordingly, in one embodiment, administered regenerative cells regenerate injured kidneys through their production of EGF. Accordingly, in one embodiment of the disclosure, regenerative regenerative cells are therapeutically useful in the treatment of kidney failure by assisting processes associated with the dedifferentiation and redifferentiation of renal cells. An alternative possibility is that a minority of surviving intratubular cells possess stem cell properties and selectively proliferate after damage to neighboring cells. In this situation the disclosure teaches that regenerative regenerative cells may act as cells capable of differentiating into renal cells and/or supporting existing cells.

In one embodiment, regenerative cells, or fibroblast-conditioned media is used in combination with one or more immune suppressive agents to augment its activity at reducing inflammation associated with kidney failure and augmented endothelin release. It will be known to one of skill in the art to choose from various immune suppressive agents. For example, some immune suppressive agents, such as anti-CD52 antibodies may be used when a systemic depletion of T and B cells is desired, whereas agents that concurrently stimulate T regulatory cell activity, such as Rapamycin, may be desired in other cases. The skilled practitioner is guided to several agents that are known in the art for causing immune suppression, which include cyclosporine, rapamycin, campath-1H, ATG, Prograf, anti IL-2r, MMF, FTY, LEA, cyclosporin A, diftitox, denileukin, levamisole, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, and trimetrexate), purine analogs (e.g., cladribine, fludarabine, 6-mercaptopurine, thiamiprine, and thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, and tegafur) fluocinolone, triaminolone, anecortave acetate, fluorometholone, medrysone, prednislone, etc. In another embodiment, the use of stem cell conditioned media may be used to potentiate an existing anti-inflammatory agent. Anti-inflammatory agents may comprise one or more agents including NSAIDs, interleukin-1 antagonists, dihydroorotate synthase inhibitors, p38 MAP kinase inhibitors, TNF-.alpha. inhibitors, TNF-alpha sequestration agents, and methotrexate. More specifically, anti-inflammatory agents may comprise one or more of, e.g., anti-TNF-.alpha., lysophylline, alpha 1-antitrypsin (AAT), interleukin-10 (IL-10), pentoxyfilline, COX-2 inhibitors, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (e.g., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), epsilon.-acetamidocaproic acid, s-adenosylmethionine, 3-amino-4-hydroxybutyric acid, amixetrine, bendazac, benzydamine, .alpha.-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, candelilla wax, alpha bisabolol, aloe vera, Manjistha, Guggal, kola extract, chamomile, sea whip extract, glycyrrhetic acid, glycyrrhizic acid, oil soluble licorice extract, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### Example

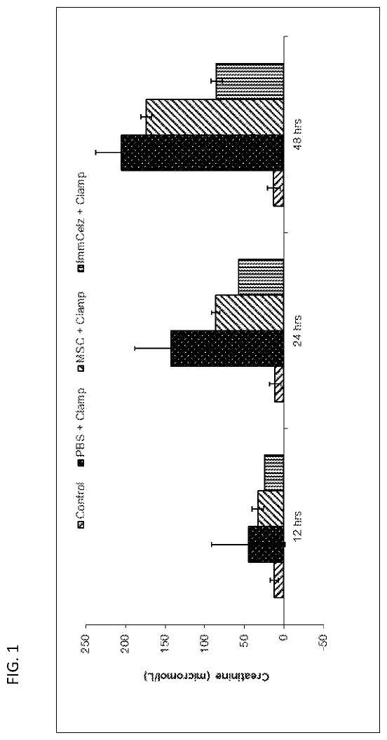

CD1 female mice of 6-8 weeks of age were anesthetized with ketamine (100 mg/kg) and xylazine (20 mg/kg) administered intraperitoneally. Renal pedicles were bluntly dissected and a microvascular clamp was placed on the left renal pedicle for 25 minutes. Following induced ischemia clamps were removed along with the right kidney. Creatinine was assessed at the reference laboratory. 10 animals had sham surgery (unclampled), 10 animals had ischemia and were treated with 500 microliters of phosphate buffered saline via tail vein (PBS), 10 animals were administered via tail vein 500,000 bone marrow mesenchymal stem cells after ischemia induction (MSC), 10 animals had received syngeneic splenocytes exposed to allogeneic bone marrow MSC (ImmCelz) administered via tail vein at a concentration of 500,000 cells. Assessment of creatinine was performed at 12, 24, and 48 hours. A significant protection from creatinine upregulation was induced by CD73 selected fibroblasts, implying renal protection by fibroblasts. The term ImmCelz™ are a proprietary type of treated T regulatory cell have regenerative properties, the disclosure of which is described in detail in U.S. Provisional Application No. 63/270,678, which was filed Oct. 22, 2021, and is hereby incorporated by reference in its Results are shown in FIG. 1

### REFERENCES

1. Ramsdell, F. and B. J. Fowlkes, Clonal deletion versus clonal anergy: the role of the thymus in inducing self tolerance. Science, 1990. 248(4961): p. 1342-8.
2. Apostolou, I., et al., Origin of regulatory T cells with known specificity for antigen. Nat Immunol, 2002. 3(8): p. 756-63.
3. Aschenbrenner, K., et al., Selection of Foxp3+ regulatory T cells specific for self antigen expressed and presented by Aire+ medullary thymic epithelial cells. Nat Immunol, 2007. 8(4): p. 351-8.
4. Cong, Y., et al., Generation of antigen-specific, Foxp3-expressing CD4+ regulatory T cells by inhibition of APC proteosome function. J Immunol, 2005. 174(5): p. 2787-95.
5. Buckland, M., et al., Aspirin-treated human DCs up-regulate ILT-3 and induce hyporesponsiveness and regulatory activity in responder T cells. Am J Transplant, 2006. 6(9): p. 2046-59.
6. Jin, Y., et al., Induction of auto-reactive regulatory T cells by stimulation with immature autologous dendritic cells. Immunol Invest, 2007. 36(2): p. 213-32.
7. Gandhi, R., D. E. Anderson, and H. L. Weiner, Cutting Edge: Immature human dendritic cells express latency-associated peptide and inhibit T cell activation in a TGF-beta-dependent manner. J Immunol, 2007. 178(7): p. 4017-21.
8. Gaudreau, S., et al., Granulocyte-macrophage colony-stimulating factor prevents diabetes development in NOD mice by inducing tolerogenic dendritic cells that sustain the suppressive function of CD4+CD25+ regulatory T cells. J Immunol, 2007. 179(6): p. 3638-47.
9. Zhang, X., et al., Generation of therapeutic dendritic cells and regulatory T cells for preventing allogeneic renal graft rejection. Clin Immunol, 2008. 127(3): p. 313-21.
10. Marguti, I., et al., Expansion of CD4+CD25+Foxp3+ T cells by bone marrow-derived dendritic cells. Immunology, 2009. 127(1): p. 50-61.
11. Vacchio, M. S. and R. J. Hodes, Fetal expression of Fas ligand is necessary and sufficient for induction of CD8 T cell tolerance to the fetal antigen H-Y during pregnancy. J Immunol, 2005. 174(8): p. 4657-61.
12. D'Addio, F., et al., The link between the PDL1 costimulatory pathway and Th17 in fetomaternal tolerance. J Immunol, 2011. 187(9): p. 4530-41.
13. Kuroki, K. and K. Maenaka, Immune modulation of HLA-G dimer in maternal-fetal interface. Eur J Immunol, 2007. 37(7): p. 1727-9.
14. Erlebacher, A., et al., Constraints in antigen presentation severely restrict T cell recognition of the allogeneic fetus. J Clin Invest, 2007. 117(5): p. 1399-411.
15. Chen, T., et al., Self-specific memory regulatory T cells protect embryos at implantation in mice. J Immunol, 2013. 191(5): p. 2273-81.
16. Harimoto, H., et al., Inactivation of tumor-specific CD8(+) CTLs by tumor-infiltrating tolerogenic dendritic cells. Immunol Cell Biol, 2013. 91(9): p. 545-55.
17. Ney, J. T., et al., Autochthonous liver tumors induce systemic T cell tolerance associated with T cell receptor down-modulation. Hepatology, 2009. 49(2): p. 471-81.
18. Cheung, A. F., et al., Regulated expression of a tumor-associated antigen reveals multiple levels of T-cell tolerance in a mouse model of lung cancer. Cancer Res, 2008. 68(22): p. 9459-68.
19. Bal, A., et al., Rapid tolerization of virus-activated tumor-specific CD8+ T cells in prostate tumors of TRAMP mice. Proc Natl Acad Sci USA, 2008. 105(35): p. 13003-8.
20. Jacobs, J. F., et al., Regulatory T cells in melanoma: the final hurdle towards effective immunotherapy? Lancet Oncol, 2012. 13(1): p. e32-42.
21. Pedroza-Gonzalez, A., et al., Activated tumor-infiltrating CD4+ regulatory T cells restrain antitumor immunity in patients with primary or metastatic liver cancer. Hepatology, 2013. 57(1): p. 183-94.
22. Donkor, M. K., et al., T cell surveillance of oncogene-induced prostate cancer is impeded by T cell-derived TGF-beta1 cytokine. Immunity, 2011. 35(1): p. 123-34.
23. Whiteside, T. L., Down-regulation of zeta-chain expression in T cells: a biomarker of prognosis in cancer? Cancer Immunol Immunother, 2004. 53(10): p. 865-78.
24. Whiteside, T. L., Signaling defects in T lymphocytes of patients with malignancy. Cancer Immunol Immunother, 1999. 48(7): p. 346-52.
25. Reichert, T. E., et al., Signaling abnormalities, apoptosis, and reduced proliferation of circulating and tumor-infiltrating lymphocytes in patients with oral carcinoma. Clin Cancer Res, 2002. 8(10): p. 3137-45.
26. Faria, A. M. and H. L. Weiner, Oral tolerance. Immunol Rev, 2005. 206: p. 232-59.
27. Weiner, H. L., Induction and mechanism of action of transforming growth factor-beta-secreting Th3 regulatory cells. Immunol Rev, 2001. 182: p. 207-14.
28. Fukaura, H., et al., Induction of circulating myelin basic protein and proteolipid protein-specific transforming growth factor-beta1-secreting Th3 T cells by oral administration of myelin in multiple sclerosis patients. J Clin Invest, 1996. 98(1): p. 70-7.
29. Palomares, O., et al., Induction and maintenance of allergen-specific FOXP3+ Treg cells in human tonsils as potential first-line organs of oral tolerance. J Allergy Clin Immunol, 2012. 129(2): p. 510-20, 520 el-9.
30. Yamashita, H., et al., Overcoming food allergy through acquired tolerance conferred by transfer of Tregs in a murine model. Allergy, 2012. 67(2): p. 201-9.
31. Park, K. S., et al., Type II collagen oral tolerance; mechanism and role in collagen-induced arthritis and rheumatoid arthritis. Mod Rheumatol, 2009.
32. Womer, K. L., et al., A pilot study on the immunological effects of oral administration of donor major histocompatibility complex class II peptides in renal transplant recipients. Clin Transplant, 2008. 22(6): p. 754-9.
33. Faria, A. M. and H. L. Weiner, Oral tolerance: therapeutic implications for autoimmune diseases. Clin Dev Immunol, 2006. 13(2-4): p. 143-57.
34. Park, M. J., et al., A distinct tolerogenic subset of splenic IDO(+)CD11b(+) dendritic cells from orally tolerized mice is responsible for induction of systemic immune tolerance and suppression of collagen-induced arthritis. Cell Immunol, 2012. 278(1-2): p. 45-54.
35. Thompson, H. S., et al., Suppression of collagen induced arthritis by oral administration of type II collagen: changes in immune and arthritic responses mediated by active peripheral suppression. Autoimmunity, 1993. 16(3): p. 189-99.
36. Song, F., et al., The thymus plays a role in oral tolerance induction in experimental autoimmune encephalomyelitis. Ann N Y Acad Sci, 2004. 1029: p. 402-4.
37. Hanninen, A. and L. C. Harrison, Mucosal tolerance to prevent type 1 diabetes: can the outcome be improved in humans? Rev Diabet Stud, 2004. 1(3): p. 113-21.
38. Wei, W., et al., A multicenter, double-blind, randomized, controlled phase III clinical trial of chicken type II collagen in rheumatoid arthritis. Arthritis Res Ther, 2009. 11(6): p. R180.
39. Thurau, S. R., et al., Molecular mimicry as a therapeutic approach for an autoimmune disease: oral treatment of uveitis-patients with an MHC-peptide crossreactive with autoantigen—first results. Immunol Lett, 1997. 57(1-3): p. 193-201.
40. Weiner, H. L., et al., Double-blind pilot trial of oral tolerization with myelin antigens in multiple sclerosis. Science, 1993. 259(5099): p. 1321-4.
41. Onishi, Y., et al., Foxp3+ natural regulatory T cells preferentially form aggregates on dendritic cells in vitro and actively inhibit their maturation. Proc Natl Acad Sci USA, 2008. 105(29): p. 10113-8.
42. Ozeri, E., et al., alpha-1 antitrypsin promotes semimature, IL-10-producing and readily migrating tolerogenic dendritic cells. J Immunol, 2012. 189(1): p. 146-53.
43. Ely, E. W., G. R. Bernard, and J. L. Vincent, Activated protein C for severe sepsis. N Engl J Med, 2002. 347(13): p. 1035-6.
44. Dhainaut, J. F., et al., Drotrecogin alfa (activated) (recombinant human activated protein C) reduces host coagulopathy response in patients with severe sepsis. Thromb Haemost, 2003. 90(4): p. 642-53.
45. Minhas, N., et al., Activated protein C utilizes the angiopoietin/Tie2 axis to promote endothelial barrier function. FASEB J. 24(3): p. 873-81.
46. Loubele, S. T., H. M. Spronk, and H. Ten Cate, Activated protein C: a promising drug with multiple effects? Mini Rev Med Chem, 2009. 9(5): p. 620-6.
47. Poole, D., G. Bertolini, and S. Garattini, Errors in the approval process and post-marketing evaluation of drotrecogin alfa (activated) for the treatment of severe sepsis. Lancet Infect Dis, 2009. 9(1): p. 67-72.
48. Pastores, S. M., et al., Septic shock and multiple organ failure after hematopoietic stem cell transplantation: treatment with recombinant human activated protein C. Bone Marrow Transplant, 2002. 30(2): p. 131-4.
49. Gando, S., Disseminated intravascular coagulation in trauma patients. Semin Thromb Hemost, 2001. 27(6): p. 585-92.
50. Cristofaro, P. and S. M. Opal, The Toll-like receptors and their role in septic shock. Expert Opin Ther Targets, 2003. 7(5): p. 603-12.
51. Treutiger, C. J., et al., High mobility group 1 B-box mediates activation of human endothelium. J Intern Med, 2003. 254(4): p. 375-85.
52. Lv, B., et al., High-mobility group box 1 protein induces tissue factor expression in vascular endothelial cells via activation of NF-kappaB and Egr-1. Thromb Haemost, 2009. 102(2): p. 352-9.
53. Wada, H., Y. Wakita, and H. Shiku, Tissue factor expression in endothelial cells in health and disease. Blood Coagul Fibrinolysis, 1995. 6 Suppl 1: p. S26-31.
54. Levi, M., The coagulant response in sepsis and inflammation. Hamostaseologie. 30(1): p. 10-2, 14-6.
55. Munro, J. M., J. S. Pober, and R. S. Cotran, Recruitment of neutrophils in the local endotoxin response: association with de novo endothelial expression of endothelial leukocyte adhesion molecule-1. Lab Invest, 1991. 64(2): p. 295-9.
56. Lippi, G., L. Ippolito, and G. Cervellin, Disseminated intravascular coagulation in burn injury. Semin Thromb Hemost. 36(4): p. 429-36.
57. Lau, C. L., et al., Enhanced fibrinolysis protects against lung ischemia-reperfusion injury. J Thorac Cardiovasc Surg, 2009. 137(5): p. 1241-8.
58. Levi, M., M. Schouten, and T. van der Poll, Sepsis, coagulation, and antithrombin: old lessons and new insights. Semin Thromb Hemost, 2008. 34(8): p. 742-6.
59. Shapiro, N., et al., The association of endothelial cell signaling, severity of illness, and organ dysfunction in sepsis. Crit Care. 14(5): p. R182.
60. Druey, K. M. and P. R. Greipp, Narrative review: the systemic capillary leak syndrome. Ann Intern Med. 153(2): p. 90-8.
61. Dejana, E., F. Orsenigo, and M. G. Lampugnani, The role of adherens junctions and VE-cadherin in the control of vascular permeability. J Cell Sci, 2008. 121(Pt 13): p. 2115-22.
62. Azevedo, L. C., et al., Redox mechanisms of vascular cell dysfunction in sepsis. Endocr Metab Immune Disord Drug Targets, 2006. 6(2): p. 159-64.
63. Okajima, K., Prevention of endothelial cell injury by activated protein C: the molecular mechanism(s) and therapeutic implications. Curr Vasc Pharmacol, 2004. 2(2): p. 125-33.
64. Andersson, U. and K. J. Tracey, HMGB1 in sepsis. Scand J Infect Dis, 2003. 35(9): p. 577-84.
65. Strassheim, D., J. S. Park, and E. Abraham, Sepsis: current concepts in intracellular signaling. Int J Biochem Cell Biol, 2002. 34(12): p. 1527-33.
66. ten Cate, H., et al., Microvascular coagulopathy and disseminated intravascular coagulation. Crit Care Med, 2001. 29(7 Suppl): p. S95-7; discussion S97-8.
67. Hawiger, J., Innate immunity and inflammation: a transcriptional paradigm. Immunol Res, 2001. 23(2-3): p. 99-109.
68. Edgington, T. S., et al., Cellular immune and cytokine pathways resulting in tissue factor expression and relevance to septic shock. Nouv Rev Fr Hematol, 1992. 34 Suppl: p. S15-27.
69. Mukaida, N., et al., Novel insight into molecular mechanism of endotoxin shock: biochemical analysis of LPS receptor signaling in a cell-free system targeting NF-kappaB and regulation of cytokine production/action through beta2 integrin in vivo. J Leukoc Biol, 1996. 59(2): p. 145-51.
70. Liu, S. F. and A. B. Malik, NF-kappa B activation as a pathological mechanism of septic shock and inflammation. Am J Physiol Lung Cell Mol Physiol, 2006. 290(4): p. L622-L645.
71. Ulfhammer, E., et al., TNF-alpha mediated suppression of tissue type plasminogen activator expression in vascular endothelial cells is NF-kappaB- and p38 MAPK-dependent. J Thromb Haemost, 2006. 4(8): p. 1781-9.
72. Xu, H., et al., Selective blockade of endothelial NF-kappaB pathway differentially affects systemic inflammation and multiple organ dysfunction and injury in septic mice. J Pathol. 220(4): p. 490-8.
73. Ding, J., et al., A pivotal role of endothelial-specific NF-kappaB signaling in the pathogenesis of septic shock and septic vascular dysfunction. J Immunol, 2009. 183(6): p. 4031-8.
74. Song, D., et al., Activation of endothelial intrinsic NF-{kappa}B pathway impairs protein C anticoagulation mechanism and promotes coagulation in endotoxemic mice. Blood, 2009. 114(12): p. 2521-9.
75. Grinnell, B. W. and D. Joyce, Recombinant human activated protein C: a system modulator of vascular function for treatment of severe sepsis. Crit Care Med, 2001. 29(7 Suppl): p. S53-60; discussion S60-1.
76. Brueckmann, M., et al., Activated protein C inhibits the release of macrophage inflammatory protein-1-alpha from THP-1 cells and from human monocytes. Cytokine, 2004. 26(3): p. 106-13.
77. Cheng, T., et al., Activated protein C blocks p53-mediated apoptosis in ischemic human brain endothelium and is neuroprotective. Nat Med, 2003. 9(3): p. 338-42.
78. van Hinsbergh, V. W., et al., Activated protein C decreases plasminogen activator-inhibitor activity in endothelial cell-conditioned medium. Blood, 1985. 65(2): p. 444-51.
79. Sakata, Y., et al., Activated protein C stimulates the fibrinolytic activity of cultured endothelial cells and decreases antiactivator activity. Proc Natl Acad Sci USA, 1985. 82(4): p. 1121-5.
80. Joyce, D. E. and B. W. Grinnell, Recombinant human activated protein C attenuates the inflammatory response in endothelium and monocytes by modulating nuclear factor-kappaB. Crit Care Med, 2002. 30(5 Suppl): p. S288-93.
81. Brueckmann, M., et al., Drotrecogin alfa (activated) inhibits NF-kappa B activation and MIP-1-alpha release from isolated mononuclear cells of patients with severe sepsis. Inflamm Res, 2004. 53(10): p. 528-33.
82. Prevosto, C., et al., Generation of CD4+ or CD8+ regulatory T cells upon mesenchymal stem cell-lymphocyte interaction. Haematologica, 2007. 92(7): p. 881-8.
83. Selmani, Z., et al., Human leukocyte antigen-G5 secretion by human mesenchymal stem cells is required to suppress T lymphocyte and natural killer function and to induce CD4+CD25highFOXP3+ regulatory T cells. Stem Cells, 2008. 26(1): p. 212-22.
84. Nasef, A., et al., Leukemia inhibitory factor: Role in human mesenchymal stem cells mediated immunosuppression. Cell Immunol, 2008. 253(1-2): p. 16-22.
85. English, K., et al., Cell contact, prostaglandin E(2) and transforming growth factor beta 1 play non-redundant roles in human mesenchymal stem cell induction of CD4+CD25(High) forkhead box P3+ regulatory T cells. Clin Exp Immunol, 2009. 156(1): p. 149-60.
86. Ghannam, S., et al., Mesenchymal stem cells inhibit human Th17 cell differentiation and function and induce a T regulatory cell phenotype. J Immunol, 2010. 185(1): p. 302-12.
87. Svobodova, E., et al., The role of mouse mesenchymal stem cells in differentiation of naive T-cells into anti-inflammatory regulatory T-cell or proinflammatory helper T-cell 17 population. Stem Cells Dev, 2012. 21(6): p. 901-10.
88. Melief, S. M., et al., Multipotent stromal cells induce human regulatory T cells through a novel pathway involving skewing of monocytes toward anti-inflammatory macrophages. Stem Cells, 2013. 31(9): p. 1980-91.
89. Luz-Crawford, P., et al., Mesenchymal stem cells generate a CD4+CD25+Foxp3+ regulatory T cell population during the differentiation process of Th1 and Th17 cells. Stem Cell Res Ther, 2013. 4(3): p. 65.
90. Kwon, M. S., et al., The immunomodulatory effects of human mesenchymal stem cells on peripheral blood mononuclear cells in ALS patients. J Neurochem, 2014. 131(2): p. 206-18.
91. Cahill, E. F., et al., Jagged-1 is required for the expansion of CD4+CD25+FoxP3+ regulatory T cells and tolerogenic dendritic cells by murine mesenchymal stromal cells. Stem Cell Res Ther, 2015. 6: p. 19.
92. Sivanathan, K. N., et al., Interleukin-17A-Induced Human Mesenchymal Stem Cells Are Superior Modulators of Immunological Function. Stem Cells, 2015. 33(9): p. 2850-63.
93. Khosravi, M., et al., Mesenchymal stem cells can induce regulatory T cells via modulating miR-126a but not miR-10a. Gene, 2017. 627: p. 327-336.
94. Miyagawa, I., et al., Induction of Regulatory T Cells and Its Regulation with Insulin-like Growth Factor/Insulin-like Growth Factor Binding Protein-4 by Human Mesenchymal Stem Cells. J Immunol, 2017. 199(5): p. 1616-1625.
95. Khosravi, M., et al., Induction of CD4(+)CD25(+)Foxp3(+) regulatory T cells by mesenchymal stem cells is associated with RUNX complex factors. Immunol Res, 2018. 66(1): p. 207-218.
96. Zhang, B., et al., Mesenchymal stromal cell exosome-enhanced regulatory T-cell production through an antigen-presenting cell-mediated pathway. Cytotherapy, 2018. 20(5): p. 687-696.
97. Khosravi, M., et al., Induction of CD4(+)CD25(+)FOXP3(+) regulatory T cells by mesenchymal stem cells is associated with modulation of ubiquitination factors and TSDR demethylation. Stem Cell Res Ther, 2018. 9(1): p. 273.
98. Chen, Q. H., et al., Mesenchymal stem cells regulate the Th17/Treg cell balance partly through hepatocyte growth factor in vitro. Stem Cell Res Ther, 2020. 11(1): p. 91.
99. Naserian, S., et al., Regulatory T cell induction by mesenchymal stem cells depends on the expression of TNFR2 by T cells. Stem Cell Res Ther, 2020. 11(1): p. 534.
100. Gonzalez-Rey, E., et al., Human adipose-derived mesenchymal stem cells reduce inflammatory and T cell responses and induce regulatory T cells in vitro in rheumatoid arthritis. Ann Rheum Dis, 2010. 69(1): p. 241-8.
101. Quaedackers, M. E., et al., Cell contact interaction between adipose-derived stromal cells and allo-activated T lymphocytes. Eur J Immunol, 2009. 39(12): p. 3436-46.
102. Engela, A. U., et al., Human adipose-tissue derived mesenchymal stem cells induce functional de-novo regulatory T cells with methylated FOXP3 gene DNA. Clin Exp Immunol, 2013. 173(2): p. 343-54.
103. Frazier, T. P., et al., Human adipose-derived stromal/stem cells induce functional CD4+CD25+FoxP3+CD127− regulatory T cells under low oxygen culture conditions. Stem Cells Dev, 2014. 23(9): p. 968-77.
104. Mohammadzadeh, A., et al., Immunomodulatory effects of adipose-derived mesenchymal stem cells on the gene expression of major transcription factors of T cell subsets. Int Immunopharmacol, 2014. 20(2): p. 316-21.
105. Vasilev, G., et al., Secretory factors produced by adipose mesenchymal stem cells downregulate Th17 and increase Treg cells in peripheral blood mononuclear cells from rheumatoid arthritis patients. Rheumatol Int, 2019. 39(5): p. 819-826.
106. Fakhimi, M., et al., Helios, CD73 and CD39 Induction in Regulatory T Cells Exposed to Adipose Derived Mesenchymal Stem Cells. Cell J, 2020. 22(2): p. 236-244.
107. Gornostaeva, A. N., et al., Adipose-derived stromal cell immunosuppression of T cells is enhanced under “physiological” hypoxia. Tissue Cell, 2020. 63: p. 101320.
108. Franco da Cunha, F., et al., Extracellular Vesicles isolated from Mesenchymal Stromal Cells Modulate CD4(+) T Lymphocytes Toward a Regulatory Profile. Cells, 2020. 9(4).
109. Azevedo, R. I., et al., Mesenchymal stromal cells induce regulatory T cells via epigenetic conversion of human conventional CD4 T cells in vitro. Stem Cells, 2020. 38(8): p. 1007-1019.
110. Bolandi, Z., et al., Adipose derived mesenchymal stem cell exosomes loaded with miR-10a promote the differentiation of Th17 and Treg from naive CD4(+) T cell. Life Sci, 2020. 259: p. 118218.
111. Bi, Y., et al., Human Adipose Tissue-Derived Mesenchymal Stem Cells in Parkinson's Disease: Inhibition of T Helper 17 Cell Differentiation and Regulation of Immune Balance Towards a Regulatory T Cell Phenotype. Clin Intery Aging, 2020. 15: p. 1383-1391.
112. Demircan, P. C., et al., Immunoregulatory effects of human dental pulp-derived stem cells on T cells: comparison of transwell co-culture and mixed lymphocyte reaction systems. Cytotherapy, 2011. 13(10): p. 1205-20.
113. Yildirim, S., et al., The Comparison of the Immunologic Properties of Stem Cells Isolated from Human Exfoliated Deciduous Teeth, Dental Pulp, and Dental Follicles. Stem Cells Int, 2016. 2016: p. 4682875.
114. Hong, J. W., et al., Immune Tolerance of Human Dental Pulp-Derived Mesenchymal Stem Cells Mediated by CD4(+)CD25(+)FoxP3(+) Regulatory T-Cells and Induced by TGF-beta1 and IL-10. Yonsei Med J, 2017. 58(5): p. 1031-1039.
115. Pfisterer, K., et al., CD90(+) human dermal stromal cells are potent inducers of FoxP3(+) regulatory T cells. J Invest Dermatol, 2015. 135(1): p. 130-141.
116. Erkers, T., et al., Decidual stromal cells promote regulatory T cells and suppress alloreactivity in a cell contact-dependent manner. Stem Cells Dev, 2013. 22(19): p. 2596-605.
117. Yi, J. Z., et al., Interferon-gamma suppresses the proliferation and migration of human placenta-derived mesenchmal stromal cells and enhances their ability to induce the generation of CD4(+)CXCR5(+)Foxp3(+) Treg subset. Cell Immunol, 2018. 326: p. 42-51.
118. Kim, S. H., et al., Immunomodulatory Effects of Placenta-derived Mesenchymal Stem Cells on T Cells by Regulation of FoxP3 Expression. Int J Stem Cells, 2018. 11(2): p. 196-204.
119. Pianta, S., et al., Amniotic membrane mesenchymal cells-derived factors skew T cell polarization toward Treg and downregulate Th1 and Th17 cells subsets. Stem Cell Rev Rep, 2015. 11(3): p. 394-407.
120. Roux, C., et al., Immunosuppressive Mesenchymal Stromal Cells Derived from Human-Induced Pluripotent Stem Cells Induce Human Regulatory T Cells In Vitro and In Vivo. Front Immunol, 2017. 8: p. 1991.
121. Darlan, D. M., et al., MSCs-released TGFbeta1 generate CD4(+)CD25(+)Foxp3(+) in T-reg cells of human SLE PBMC. J Formos Med Assoc, 2021. 120(1 Pt 3): p. 602-608.
122. Han, K. H., et al., Immunosuppressive mechanisms of embryonic stem cells and mesenchymal stem cells in alloimmune response. Transpl Immunol, 2011. 25(1): p. 7-15.
123. Xiao, Y., et al., Efficacy and safety of mesenchymal stromal cell treatment from related donors for patients with refractory aplastic anemia. Cytotherapy, 2013. 15(7): p. 760-6.
124. Li, H., et al., In patients with chronic aplastic anemia, bone marrow-derived MSCs regulate the Treg/Th17 balance by influencing the Notch/RBP-J/FOXP3/RORgammat pathway. Sci Rep, 2017. 7: p. 42488.
125. Casiraghi, F., et al., Pretransplant infusion of mesenchymal stem cells prolongs the survival of a semiallogeneic heart transplant through the generation of regulatory T cells. J Immunol, 2008. 181(6): p. 3933-46.
126. Ge, W., et al., Infusion of mesenchymal stem cells and rapamycin synergize to attenuate alloimmune responses and promote renal allograft tolerance. Am J Transplant, 2009. 9(8): p. 1760-72.
127. Wang, Y., et al., Bone marrow-derived mesenchymal stem cells inhibit acute rejection of rat liver allografts in association with regulatory T-cell expansion. Transplant Proc, 2009. 41(10): p. 4352-6.
128. Ge, W., et al., Regulatory T-cell generation and kidney allograft tolerance induced by mesenchymal stem cells associated with indoleamine 2,3-dioxygenase expression. Transplantation, 2010. 90(12): p. 1312-20.
129. Kim, Y. H., et al., Interleukin (IL)-10 induced by CD11b(+) cells and IL-10-activated regulatory T cells play a role in immune modulation of mesenchymal stem cells in rat islet allografts. Mol Med, 2011. 17(7-8): p. 697-708.
130. Kuo, Y. R., et al., Immunomodulatory effects of bone marrow-derived mesenchymal stem cells in a swine hemi-facial allotransplantation model. PLoS One, 2012. 7(4): p. e35459.
131. Perico, N., et al., Mesenchymal stromal cells and kidney transplantation: pretransplant infusion protects from graft dysfunction while fostering immunoregulation. Transpl Int, 2013. 26(9): p. 867-78.
132. Huang, H., et al., Combined intrathymic and intravenous injection of mesenchymal stem cells can prolong the survival of rat renal allograft associated with decrease in miR-155 expression. J Surg Res, 2013. 185(2): p. 896-903.
133. Lee, J. H., et al., The synergistic immunoregulatory effects of culture-expanded mesenchymal stromal cells and CD4(+)25(+)Foxp3+ regulatory T cells on skin allograft rejection. PLoS One, 2013. 8(8): p. e70968.
134. Zhao, Y., et al., Treatment with placenta-derived mesenchymal stem cells mitigates development of bronchiolitis obliterans in a murine model. J Thorac Cardiovasc Surg, 2014. 147(5): p. 1668-1677 e5.
135. Niu, J., et al., Prevention of acute liver allograft rejection by IL-10-engineered mesenchymal stem cells. Clin Exp Immunol, 2014. 176(3): p. 473-84.
136. Wang, H., et al., Requirement of B7-H1 in mesenchymal stem cells for immune tolerance to renal allografts in combination therapy with rapamycin. Transpl Immunol, 2014. 31(2): p. 65-74.
137. Mudrabettu, C., et al., Safety and efficacy of autologous mesenchymal stromal cells transplantation in patients undergoing living donor kidney transplantation: a pilot study. Nephrology (Carlton), 2015. 20(1): p. 25-33.
138. Gregorini, M., et al., Mesenchymal stromal cells reset the scatter factor system and cytokine network in experimental kidney transplantation. BMC Immunol, 2014. 15: p. 44.
139. Treacy, O., et al., Mesenchymal stem cell therapy promotes corneal allograft survival in rats by local and systemic immunomodulation. Am J Transplant, 2014. 14(9): p. 2023-36.
140. Takahashi, T., et al., Multipotent mesenchymal stromal cells synergize with costimulation blockade in the inhibition of immune responses and the induction of Foxp3+ regulatory T cells. Stem Cells Transl Med, 2014. 3(12): p. 1484-94.
141. Qi, H., et al., Foxp3-modified bone marrow mesenchymal stem cells promotes liver allograft tolerance through the generation of regulatory T cells in rats. J Transl Med, 2015. 13: p. 274.
142. He, Y., et al., Indoleamine 2, 3-Dioxgenase Transfected Mesenchymal Stem Cells Induce Kidney Allograft Tolerance by Increasing the Production and Function of Regulatory T Cells. Transplantation, 2015. 99(9): p. 1829-38.
143. Srivastava, A. K., et al., Co-transplantation of syngeneic mesenchymal stem cells improves survival of allogeneic glial-restricted precursors in mouse brain. Exp Neurol, 2016. 275 Pt 1: p. 154-61.
144. Tang, J., et al., Transforming growth factor-beta-Expressing Mesenchymal Stem Cells Induce Local Tolerance in a Rat Liver Transplantation Model of Acute Rejection. Stem Cells, 2016. 34(11): p. 2681-2692.
145. Liu, T., et al., Immunomodulatory effects of OX40Ig gene-modified adipose tissue-derived mesenchymal stem cells on rat kidney transplantation. Int J Mol Med, 2017. 39(1): p. 144-152.
146. Kuo, Y. R., et al., Recipient Adipose-Derived Stem Cells Enhance Recipient Cell Engraftment and Prolong Allotransplant Survival in a Miniature Swine Hind-Limb Model. Cell Transplant, 2017. 26(8): p. 1418-1427.
147. Lan, X., et al., Stromal Cell-Derived Factor-1 Mediates Renal Allograft Tolerance Induced by Human Endometrial Regenerative Cell-Based Therapy. Stem Cells Transl Med, 2017. 6(11): p. 1997-2008.
148. Hua, F., et al., Protective action of bone marrow mesenchymal stem cells in immune tolerance of allogeneic heart transplantation by regulating CD45RB(+) dendritic cells. Clin Transplant, 2018. 32(4): p. e13231.
149. Ye, K., et al., B7-H1 Expression Is Required for Human Endometrial Regenerative Cells in the Prevention of Transplant Vasculopathy in Mice. Stem Cells Int, 2018. 2018: p. 2405698.
150. Niu, J., et al., Mesenchymal stem cells prolong the survival of orthotopic liver transplants by regulating the expression of TGF-beta1. Turk J Gastroenterol, 2018. 29(5): p. 601-609.
151. Lohan, P., et al., Third-Party Allogeneic Mesenchymal Stromal Cells Prevent Rejection in a Pre-sensitized High-Risk Model of Corneal Transplantation. Front Immunol, 2018. 9: p. 2666.
152. Joo, S. Y., et al., Mesenchymal stromal cells inhibit graft-versus-host disease of mice in a dose-dependent manner. Cytotherapy, 2010. 12(3): p. 361-70.
153. Xia, X., et al., Mesenchymal stem cells administered after liver transplantation prevent acute graft-versus-host disease in rats. Liver Transpl, 2012. 18(6): p. 696-706.
154. Lim, J. Y., et al., Combination cell therapy using mesenchymal stem cells and regulatory T-cells provides a synergistic immunomodulatory effect associated with reciprocal regulation of TH1/TH2 and th17/treg cells in a murine acute graft-versus-host disease model. Cell Transplant, 2014. 23(6): p. 703-14.
155. Zhao, K., et al., Immunomodulation effects of mesenchymal stromal cells on acute graft-versus-host disease after hematopoietic stem cell transplantation. Biol Blood Marrow Transplant, 2015. 21(1): p. 97-104.
156. Yu, M., et al., High expression of heme oxygenase-1 in target organs may attenuate acute graft-versus-host disease through regulation of immune balance of TH17/Treg. Transpl Immunol, 2016. 37: p. 10-17.
157. Ni, X., et al., Reduction in murine acute GVHD severity by human gingival tissue-derived mesenchymal stem cells via the CD39 pathways. Cell Death Dis, 2019. 10(1): p. 13.
158. Zhu, H., et al., Epidermal growth factor stimulates exosomal microRNA-21 derived from mesenchymal stem cells to ameliorate aGVHD by modulating regulatory T cells. FASEB J, 2020. 34(6): p. 7372-7386.
159. Kavanagh, H. and B. P. Mahon, Allogeneic mesenchymal stem cells prevent allergic airway inflammation by inducing murine regulatory T cells. Allergy, 2011. 66(4): p. 523-31.
160. Li, J. G., et al., Human mesenchymal stem cells elevate CD4+CD25+CD127low/− regulatory T cells of asthmatic patients via heme oxygenase-1. Iran J Allergy Asthma Immunol, 2013. 12(3): p. 228-35.
161. Dai, R., et al., Delivery of adipose-derived mesenchymal stem cells attenuates airway responsiveness and inflammation in a mouse model of ovalbumin-induced asthma. Am J Transl Res, 2017. 9(5): p. 2421-2428.
162. Li, Y., et al., Placenta derived mesenchymal stem cells improve airway hyperresponsiveness and inflammation in asthmatic rats by modulating the Th17/Treg balance. Mol Med Rep, 2017. 16(6): p. 8137-8145.
163. Dai, R., et al., Intratracheal administration of adipose derived mesenchymal stem cells alleviates chronic asthma in a mouse model. BMC Pulm Med, 2018. 18(1): p. 131.
164. Boumaza, I., et al., Autologous bone marrow-derived rat mesenchymal stem cells promote PDX-1 and insulin expression in the islets, alter T cell cytokine pattern and preserve regulatory T cells in the periphery and induce sustained normoglycemia. J Autoimmun, 2009. 32(1): p. 33-42.
165. Madec, A. M., et al., Mesenchymal stem cells protect NOD mice from diabetes by inducing regulatory T cells. Diabetologia, 2009. 52(7): p. 1391-9.
166. Hu, J., et al., Effect and mechanisms of human Wharton's jelly-derived mesenchymal stem cells on type 1 diabetes in NOD model. Endocrine, 2015. 48(1): p. 124-34.
167. Favaro, E., et al., Human mesenchymal stem cell-derived microvesicles modulate T cell response to islet antigen glutamic acid decarboxylase in patients with type 1 diabetes. Diabetologia, 2014. 57(8): p. 1664-73.
168. Favaro, E., et al., Human mesenchymal stem cells and derived extracellular vesicles induce regulatory dendritic cells in type 1 diabetic patients. Diabetologia, 2016. 59(2): p. 325-33.
169. Jung, K. H., et al., Human bone marrow-derived clonal mesenchymal stem cells inhibit inflammation and reduce acute pancreatitis in rats. Gastroenterology, 2011. 140(3): p. 998-1008.
170. Mohajeri, M., et al., FOXP3 gene expression in multiple sclerosis patients pre- and post mesenchymal stem cell therapy. Iran J Allergy Asthma Immunol, 2011. 10(3): p. 155-61.
171. Zhu, J., et al., Transplanting of mesenchymal stem cells may affect proliferation and function of CD4(+) T cells in experimental autoimmune encephalomyelitis. Exp Clin Transplant, 2012. 10(5): p. 492-500.
172. Yousefi, F., et al., Comparison of in vivo immunomodulatory effects of intravenous and intraperitoneal administration of adipose-tissue mesenchymal stem cells in experimental autoimmune encephalomyelitis (EAE). Int Immunopharmacol, 2013. 17(3): p. 608-16.
173. Trubiani, O., et al., Alternative source of stem cells derived from human periodontal ligament: a new treatment for experimental autoimmune encephalomyelitis. Stem Cell Res Ther, 2016. 7: p. 1.
174. Yousefi, F., et al., In vivo immunomodulatory effects of adipose-derived mesenchymal stem cells conditioned medium in experimental autoimmune encephalomyelitis. Immunol Lett, 2016. 172: p. 94-105.
175. Kurte, M., et al., IL17/IL17RA as a Novel Signaling Axis Driving Mesenchymal Stem Cell Therapeutic Function in Experimental Autoimmune Encephalomyelitis. Front Immunol, 2018. 9: p. 802.
176. Kim, M. J., et al., Combined Treatment with Methylprednisolone and Human Bone Marrow-Derived Mesenchymal Stem Cells Ameliorate Experimental Autoimmune Encephalomyelitis. Tissue Eng Regen Med, 2018. 15(2): p. 183-194.
177. Jafarinia, M., et al., Therapeutic effects of extracellular vesicles from human adipose-derived mesenchymal stem cells on chronic experimental autoimmune encephalomyelitis. J Cell Physiol, 2020. 235(11): p. 8779-8790.
178. Wang, D., et al., Effect of allogeneic bone marrow-derived mesenchymal stem cells transplantation in a polyI:C-induced primary biliary cirrhosis mouse model. Clin Exp Med, 2011. 11(1): p. 25-32.
179. Wang, L., et al., Allogeneic bone marrow mesenchymal stem cell transplantation in patients with UDCA-resistant primary biliary cirrhosis. Stem Cells Dev, 2014. 23(20): p. 2482-9.
180. Xu, L., et al., Randomized trial of autologous bone marrow mesenchymal stem cells transplantation for hepatitis B virus cirrhosis: regulation of Treg/Th17 cells. J Gastroenterol Hepatol, 2014. 29(8): p. 1620-8.
181. Lu, S., et al., Human endometrial regenerative cells alleviate carbon tetrachloride-induced acute liver injury in mice. J Transl Med, 2016. 14(1): p. 300.
182. Milosavljevic, N., et al., Mesenchymal stem cells attenuate acute liver injury by altering ratio between interleukin 17 producing and regulatory natural killer T cells. Liver Transpl, 2017. 23(8): p. 1040-1050.
183. Milosavljevic, N., et al., Mesenchymal stem cells attenuate liver fibrosis by suppressing Th17 cells—an experimental study. Transpl Int, 2018. 31(1): p. 102-115.
184. Gonzalez, M. A., et al., Adipose-derived mesenchymal stem cells alleviate experimental colitis by inhibiting inflammatory and autoimmune responses. Gastroenterology, 2009. 136(3): p. 978-89.
185. Chen, Q. Q., et al., Mesenchymal stem cells alleviate TNBS-induced colitis by modulating inflammatory and autoimmune responses. World J Gastroenterol, 2013. 19(29): p. 4702-17.
186. Kim, H. S., et al., Human umbilical cord blood mesenchymal stem cells reduce colitis in mice by activating NOD2 signaling to COX2. Gastroenterology, 2013. 145(6): p. 1392-403 el-8.
187. Zuo, D., et al., Study on the interactions between transplanted bone marrow-derived mesenchymal stem cells and regulatory T cells for the treatment of experimental colitis. Int J Mol Med, 2013. 32(6): p. 1337-44.
188. Lv, Y., et al., Endometrial regenerative cells as a novel cell therapy attenuate experimental colitis in mice. J Transl Med, 2014. 12: p. 344.
189. Sala, E., et al., Mesenchymal Stem Cells Reduce Colitis in Mice via Release of TSG6, Independently of Their Localization to the Intestine. Gastroenterology, 2015. 149(1): p. 163-176 e20.
190. Wang, M., et al., Intraperitoneal injection (IP), Intravenous injection (IV) or anal injection (AI)? Best way for mesenchymal stem cells transplantation for colitis. Sci Rep, 2016. 6: p. 30696.
191. Cheng, W., et al., Interleukin-25 primed mesenchymal stem cells achieve better therapeutic effects on dextran sulfate sodium-induced colitis via inhibiting Th17 immune response and inducing T regulatory cell phenotype. Am J Transl Res, 2017. 9(9): p. 4149-4160.
192. Yan, Y., et al., Mesenchymal stem cell expression of interleukin-35 protects against ulcerative colitis by suppressing mucosal immune responses. Cytotherapy, 2018. 20(7): p. 911-918.
193. An, J. H., et al., Prostaglandin E2 secreted from feline adipose tissue-derived mesenchymal stem cells alleviate DSS-induced colitis by increasing regulatory T cells in mice. BMC Vet Res, 2018. 14(1): p. 354.
194. Gonzalez, M. A., et al., Treatment of experimental arthritis by inducing immune tolerance with human adipose-derived mesenchymal stem cells. Arthritis Rheum, 2009. 60(4): p. 1006-19.
195. Park, M. J., et al., Transforming growth factor beta-transduced mesenchymal stem cells ameliorate experimental autoimmune arthritis through reciprocal regulation of Treg/Th17 cells and osteoclastogenesis. Arthritis Rheum, 2011. 63(6): p. 1668-80.
196. Chen, M., et al., Adoptive transfer of human gingiva-derived mesenchymal stem cells ameliorates collagen-induced arthritis via suppression of Th1 and Th17 cells and enhancement of regulatory T cell differentiation. Arthritis Rheum, 2013. 65(5): p. 1181-93.
197. Wang, L., et al., Human umbilical cord mesenchymal stem cell therapy for patients with active rheumatoid arthritis: safety and efficacy. Stem Cells Dev, 2013. 22(24): p. 3192-202.
198. Choi, E. W., et al., Effects of Transplantation of CTLA4Ig-Overexpressing Adipose Tissue-Derived Mesenchymal Stem Cells in Mice With Sustained Severe Rheumatoid Arthritis. Cell Transplant, 2016. 25(2): p. 243-59.
199. Chen, Y., et al., Gene delivery with IFN-gamma-expression plasmids enhances the therapeutic effects of MSCs on DSS-induced mouse colitis. Inflamm Res, 2015. 64(9): p. 671-81.
200. Lopez-Santalla, M., et al., Human Adipose-Derived Mesenchymal Stem Cells Modulate Experimental Autoimmune Arthritis by Modifying Early Adaptive T Cell Responses. Stem Cells, 2015. 33(12): p. 3493-503.
201. Lim, J. Y., et al., Enhanced immunoregulation of mesenchymal stem cells by IL-10-producing type 1 regulatory T cells in collagen-induced arthritis. Sci Rep, 2016. 6: p. 26851.
202. Sun, Y., et al., Comparable therapeutic potential of umbilical cord mesenchymal stem cells in collagen-induced arthritis to TNF inhibitor or anti-CD20 treatment. Clin Exp Rheumatol, 2017. 35(2): p. 288-295.
203. Mancheno-Corvo, P., et al., Intralymphatic Administration of Adipose Mesenchymal Stem Cells Reduces the Severity of Collagen-Induced Experimental Arthritis. Front Immunol, 2017. 8: p. 462.
204. Yan, M., et al., Intra-Articular Injection of Human Synovial Membrane-Derived Mesenchymal Stem Cells in Murine Collagen-Induced Arthritis: Assessment of Immunomodulatory Capacity In Vivo. Stem Cells Int, 2017. 2017: p. 9198328.
205. Kay, A. G., et al., Mesenchymal Stem Cell-Conditioned Medium Reduces Disease Severity and Immune Responses in Inflammatory Arthritis. Sci Rep, 2017. 7(1): p. 18019.
206. Nam, Y., et al., Intraperitoneal infusion of mesenchymal stem cell attenuates severity of collagen antibody induced arthritis. PLoS One, 2018. 13(6): p. e0198740.
207. Pers, Y. M., et al., Injection of Adipose-Derived Stromal Cells in the Knee of Patients with Severe Osteoarthritis has a Systemic Effect and Promotes an Anti-Inflammatory Phenotype of Circulating Immune Cells. Theranostics, 2018. 8(20): p. 5519-5528.
208. Ghoryani, M., et al., The Sufficient Immunoregulatory Effect of Autologous Bone Marrow-Derived Mesenchymal Stem Cell Transplantation on Regulatory T Cells in Patients with Refractory Rheumatoid Arthritis. J Immunol Res, 2020. 2020: p. 3562753.
209. Carrion, F., et al., Autologous mesenchymal stem cell treatment increased T regulatory cells with no effect on disease activity in two systemic lupus erythematosus patients. Lupus, 2010. 19(3): p. 317-22.
210. Choi, E. W., et al., Reversal of serologic, immunologic, and histologic dysfunction in mice with systemic lupus erythematosus by long-term serial adipose tissue-derived mesenchymal stem cell transplantation. Arthritis Rheum, 2012. 64(1): p. 243-53.
211. Wang, Q., et al., Combined transplantation of autologous hematopoietic stem cells and allogenic mesenchymal stem cells increases T regulatory cells in systemic lupus erythematosus with refractory lupus nephritis and leukopenia. Lupus, 2015. 24(11): p. 1221-6.
212. Wang, D., et al., The regulation of the Treg/Th17 balance by mesenchymal stem cells in human systemic lupus erythematosus. Cell Mol Immunol, 2017. 14(5): p. 423-431.
213. He, X., et al., Suppression of interleukin 17 contributes to the immunomodulatory effects of adipose-derived stem cells in a murine model of systemic lupus erythematosus. Immunol Res, 2016. 64(5-6): p. 1157-1167.
214. Chen, C., et al., Mesenchymal stem cells upregulate Treg cells via sHLA-G in SLE patients. Int Immunopharmacol, 2017. 44: p. 234-241.
215. Zhang, X., et al., Mesenchymal stem cells ameliorate experimental autoimmune uveoretinitis by comprehensive modulation of systemic autoimmunity. Invest Ophthalmol Vis Sci, 2011. 52(6): p. 3143-52.
216. Oh, J. Y., et al., Intraperitoneal infusion of mesenchymal stem/stromal cells prevents experimental autoimmune uveitis in mice. Mediators Inflamm, 2014. 2014: p. 624640.
217. Zhou, Y., et al., The therapeutic efficacy of human adipose tissue-derived mesenchymal stem cells on experimental autoimmune hearing loss in mice. Immunology, 2011. 133(1): p. 133-40.
218. Yoo, T. J., X. Du, and B. Zhou, The paracrine effect of mesenchymal human stem cells restored hearing in beta-tubulin induced autoimmune sensorineural hearing loss. Hear Res, 2015. 330(Pt A): p. 57-61.
219. Choi, E. W., et al., Therapeutic effects of CTLA4Ig gene-transduced adipose tissue-derived mesenchymal stem cell transplantation on established autoimmune thyroiditis. Cell Transplant, 2015. 24(11): p. 2221-36.
220. Sun, J., et al., Intrapulmonary delivery of human umbilical cord mesenchymal stem cells attenuates acute lung injury by expanding CD4+CD25+ Forkhead Boxp3 (FOXP3)+ regulatory T cells and balancing anti- and pro-inflammatory factors. Cell Physiol Biochem, 2011. 27(5): p. 587-96.
221. Gore, A. V., et al., Mesenchymal stem cells enhance lung recovery after injury, shock, and chronic stress. Surgery, 2016. 159(5): p. 1430-5.
222. Kim, M. G., et al., CD11c(+) cells partially mediate the renoprotective effect induced by bone marrow-derived mesenchymal stem cells. PLoS One, 2013. 8(8): p. e72544.
223. Moghadasali, R., et al., Intra-renal arterial injection of autologous bone marrow mesenchymal stromal cells ameliorates cisplatin-induced acute kidney injury in a rhesus Macaque mulatta monkey model. Cytotherapy, 2014. 16(6): p. 734-49.
224. Bai, M., et al., IL-17A improves the efficacy of mesenchymal stem cells in ischemic-reperfusion renal injury by increasing Treg percentages by the COX-2/PGE2 pathway. Kidney Int, 2018. 93(4): p. 814-825.
225. Song, K., et al., Effects of human adipose-derived mesenchymal stem cells combined with estrogen on regulatory T cells in patients with premature ovarian insufficiency. Int Immunopharmacol, 2018. 55: p. 257-262.

1. A method of preventing, and/or inhibiting, and/or reversing renal failure comprising the steps of: a) identifying a patient suffering from renal failure and/or a patient having undergoing renal fibrosis; b) extracting from said patient immune cells; c) contacting said immune cells with regenerative cells in a manner so that regenerative cells endow onto said immune cells properties capable of inhibiting and/or reversing renal failure and/or renal fibrosis; and d) administering said immune cells into said patient.

2. The method of claim 1, wherein said regenerative cell is a mesenchymal stem cell.

3. The method of claim 2, wherein said mesenchymal stem cells are naturally occurring mesenchymal stem cells.

4. The method of claim 2, wherein said mesenchymal stem cells are generated in vitro from pluripotent stem cells.

5. The method of claim 4, wherein said pluripotent stem cells are inducible pluripotent stem cells.

6. The method of claim 4, wherein said pluripotent stem cells are inducible parthenogenesis derived stem cells.

7. The method of claim 2, wherein said naturally occurring mesenchymal stem cells are tissue derived.

8. The method of claim 7, wherein said naturally occurring mesenchymal stem cells are derived from a bodily fluid.

9. The method of claim 8, wherein said bodily fluid is selected from a group comprising of: a) follicular fluid; b) amniotic fluid; c) blood; d) cerebrospinal fluid and e) mobilized peripheral blood.

10. The method of claim 9, wherein said mobilization of peripheral blood is accomplished by administration to the donor an agent selected from a group comprising of: a) GM-CSF; b) G-CSF; c) NGF; and d) BDNF.

11. The method of claim 7, wherein tissue derived mesenchymal stem cells are selected from a group comprising of: a) bone marrow; b) perivascular tissue; c) adipose tissue; d) placental tissue; e) amniotic membrane; f) omentum; g) tooth; h) umbilical cord tissue; i) fallopian tube tissue; j) hepatic tissue; k) renal tissue; 1) renal tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; v) skeletal muscle tissue; and w) subepithelial umbilical cord tissue.

12. The method of claim 1, wherein said patient immune cells are T cells.

13. The method of claim 1, wherein said patient immune cells are T regulatory cells.

14. The method of claim 1, wherein said contacting immune cells with said regenerative cells is exposing immune cells to conditioned media of regenerative cells.

15. The method of claim 1, wherein said regenerative cells are activated prior to obtaining conditioned media from said regenerative cells.

16. The method of claim 15, wherein said activation of said regenerative cells is contacting said cells with an agent that stimulates degradation of IKK-beta.

17. The method of claim 16, wherein said agent that stimulates said degradation of IKK-beta is interferon gamma.

18. The method of claim 15, wherein said activation is treatment of mesenchymal stem cells with 100 IU of interferon gamma per 1-10,000,000 cells for a period of 2-96 hours.

19. The method of claim 1, wherein said immune cells are treated with anti-CD3 before re-administration.

20. The method of claim 1, wherein said immune cells are subjected to hypoxia before re-administration.
